(19) United States
(12) Patent Application Publication
(10) Pub. No.: US 2024/0252536 A1
(43) Pub. Date: Aug. 1, 2024

(54) CHIMERIC ANTIGEN RECEPTORS AND MODIFIED CELLS COMPRISING THE SAME

(71) Applicant: Singapore Health Services Pte Ltd, Singpapore (SG)

(72) Inventor: Salvatore ALBANI, Singapore (SG)

(73) Assignee: Singapore Health Services Pte Ltd, Singapore (SG)

(21) Appl. No.: 18/290,242

(22) PCT Filed: May 11, 2022
(86) PCT No.: PCT/SG2022/050308

§ 371 Date: Nov. 10, 2023

(30) Foreign Application Priority Data
May 11, 2021 (SG) 10202104940X

(51) Int. Cl. A61K 35/17; A61K 39/00; C07K 14/725; C07K 16/24; C12N 5/0783; C12N 15/86

(52) U.S. Cl. CPC A61K 35/17; A61K 39/4611; A61K 39/4621; A61K 39/4631; A61K 39/464438; A61K 39/46444; A61K 39/464441; C07K 14/7051; C07K 16/241; C07K 16/244; C07K 16/249; C12N 5/0637; C12N 15/86; A61K 2039/5156; A61K 2239/13; C07K 2317/622; C07K 2319/03; C12N 2510/00; C12N 2740/15041

(57) ABSTRACT

The present invention relates to a chimeric antigen receptor (CAR) comprising an extracellular domain that binds to one or more inflammation associated factor(s), a transmembrane/hinge domain and an intracellular domain. In a specific embodiment, a T regulatory (Treg) cell expressing a construct encoding said CAR which further comprising a Fox3p and a CAR activation-dependent NF-AT promoter that drives the expression of interleukin-10 and TGF-beta. It also relate to the methods of producing the CAR, the Treg cells expressing said CAR and the use of said CAR to treat autoimmune diseases such as transplant rejection, a graft versus host disease (GVHD) and cytokine release syndrome..

### RELATED APPLICATIONS

This application derives entitlement from Singaporean provisional no 10202104940X filed 11 May 2021, the entire disclosure of which is incorporated herein by reference.

### FIELD OF THE INVENTION

The present invention refers to the field of biotechnology and bioengineering. In particular, the present invention refers to chimeric antigen receptors and modified cells, for example modified T cells, comprising the same.

### BACKGROUND OF THE INVENTION

Inflammatory diseases or conditions are a major problem worldwide and can be caused by or associated with proinflammatory cytokines.

Solid organ transplantation is currently the treatment of choice for end-stage organ failure. Early-stage graft survival has been ameliorated by better matching of donor and recipients, and improved immunosuppressive drug regimes. However, long term usage of immunosuppressive drugs has been shown to be linked to detrimental side effects.

Similar to transplantation, immunosuppressive drug administration in patients with autoimmune disorders aim to target the immune and autoimmune responses, leaving them immunocompromised.

Therefore, new or improved therapies to modulate immune activities and responses associated with inflammatory diseases or conditions, allogenic transplantations and/or autoimmune diseases or conditions are needed.

### SUMMARY

In one aspect, the present disclosure refers to a chimeric antigen receptor (CAR) comprising an extracellular domain, a transmembrane domain, and an intracellular domain, wherein the extracellular domain binds one or more inflammation associated factor(s).

In specific examples, the inflammation associated factor(s) are pro-inflammatory cytokines or inflammation—associated chemokines. In a specific example, the one or more inflammation associated factors is/are selected from the group consisting of Interleukin 1 (IL-1), Tumour Necrosis Factor alpha (TNF-alpha), Interferon gamma (IFN-gamma), Interleukin 12 (IL-12), Interleukin 18 (IL-18), Granulocyte-macrophage colony-stimulating factor (GMCSF), Interleukin 6 (IL-6), Interleukin 8 (IL-8), Interleukin 17A (IL-17), CXCL1, CXCL2, CXCL9, CXCL10, CXCL11, CXCL16, and CCL2-20.

In a specific example, the present disclosure refers to a chimeric antigen receptor (CAR) comprising an extracellular domain, a transmembrane domain, and an intracellular domain, wherein the extracellular domain binds one or more pro-inflammatory cytokines. In a further example, the one or more pro-inflammatory cytokines is/are selected from the group consisting of Interleukin 1 (IL-1), Tumour Necrosis Factor alpha (TNF-alpha), Interferon gamma (IFN-gamma), Interleukin 12 (IL-12), Interleukin 18 (IL-18), Granulocyte-macrophage colony-stimulating factor (GMCSF), Interleukin 6 (IL-6), Interleukin 8 (IL-8), and Interleukin 17A (IL-17). In one example, the pro-inflammatory cytokine is Tumour Necrosis Factor alpha (TNF-alpha). In one example, the pro-inflammatory cytokine is Interferon gamma (IFN-gamma). In one example, the pro-inflammatory cytokine is Interleukin 8 (IL-8).

In an example, the extracellular domain of the chimeric antigen receptor as disclosed herein comprises one or more antigen binding domains, wherein the/each antigen binding domain is independently selected from the group consisting of an antibody, an antibody fragment, a single-chain variable fragment (scFv), a chemokine receptor, or functional variations thereof. In one specific example, the chemokine receptor is further selected from the group consisting of CCR5, CXCR3, CCR1, and CCR2. In another specific example, the/each antigen binding domain is a single-chain variable fragment (scFv). In a specific example, the extracellular domain comprises any one or more of the below: a scFv which binds TNF-alpha, a scFv which binds IFN-gamma; and a scFv which binds IL-17a.

In certain embodiments, the antigen binding domain competes for binding to an inflammation associated factor(s) with an antibody as defined in Table 1 below.

In one embodiment, the antigen binding domain competes with an antibody comprising SEQ ID NO: 1; 19 and 20; 64; 65 and 66; 80 and 81; 85 and 86; 162 and 163; or 165 for binding to TNF-alpha.

In one embodiment, the antigen binding domain competes with an antibody comprising SEQ ID NO: 3; 5; 34 and 35; 49 and 50; 100 and 101; or 112 and 113 for binding to IFN-gamma.

In one embodiment, the antigen binding domain competes with an antibody comprising SEQ ID NO: 127 and 128; or 141 and 142 for binding to IL-17a.

In One Embodiment, the Antigen Recognition Domain Comprises:
(a) a light chain variable domain (VL) comprising three complementarity determining regions (CDRs) of the amino acid sequence shown in SEQ ID NO: 20, 35, 50, 66, 81, 86, 101, 113, 128, 142 or 163, or any VL as shown in Table 1; and
(b) a heavy chain variable domain (VH) comprising three complementarity determining regions (CDRs) of the amino acid sequence shown in SEQ ID NO: 19, 34, 49, 65, 80, 85, 100, 112, 127, 141 or 162 or any VH as shown in Table 1.

In one embodiment, the antigen recognition domain comprises:
(a) a light chain variable domain (VL) comprising three complementarity determining regions (CDRs) of the amino acid sequence shown in SEQ ID NO: 20 or SEQ ID NO: 163; and
(b) a heavy chain variable domain (VH) comprising three complementarity determining regions (CDRs) of the amino acid sequence shown in SEQ ID NO: 19 or SEQ ID NO: 162.

In one embodiment, the antigen recognition domain comprises:
(a) a light chain variable domain (VL) comprising three complementarity determining regions (CDRs) of the amino acid sequence shown in SEQ ID NO: 35; and
(b) a heavy chain variable domain (VH) comprising three complementarity determining regions (CDRs) of the amino acid sequence shown in SEQ ID NO: 34.

In one embodiment, the antigen recognition domain comprises:
(a) a light chain variable domain (VL) comprising three complementarity determining regions (CDRs) of the amino acid sequence shown in SEQ ID NO: 50; and
(b) a heavy chain variable domain (VH) comprising three complementarity determining regions (CDRs) of the amino acid sequence shown in SEQ ID NO: 49.

In one embodiment, the antigen recognition domain comprises:
(a) a light chain variable domain (VL) comprising three complementarity determining regions (CDRs) of the amino acid sequence shown in SEQ ID NO: 66; and
(b) a heavy chain variable domain (VH) comprising three complementarity determining regions (CDRs) of the amino acid sequence shown in SEQ ID NO: 65.

In one embodiment, the antigen recognition domain comprises:
(a) a light chain variable domain (VL) comprising three complementarity determining regions (CDRs) of the amino acid sequence shown in SEQ ID NO: 81; and
(b) a heavy chain variable domain (VH) comprising three complementarity determining regions (CDRs) of the amino acid sequence shown in SEQ ID NO: 80.

In one embodiment, the antigen recognition domain comprises:
(a) a light chain variable domain (VL) comprising three complementarity determining regions (CDRs) of the amino acid sequence shown in SEQ ID NO: 86; and
(b) a heavy chain variable domain (VH) comprising three complementarity determining regions (CDRs) of the amino acid sequence shown in SEQ ID NO: 85.

In one embodiment, the antigen recognition domain comprises:
(a) a light chain variable domain (VL) comprising three complementarity determining regions (CDRs) of the amino acid sequence shown in SEQ ID NO: 101; and
(b) a heavy chain variable domain (VH) comprising three complementarity determining regions (CDRs) of the amino acid sequence shown in SEQ ID NO: 100.

In one embodiment, the antigen recognition domain comprises:
(a) a light chain variable domain (VL) comprising three complementarity determining regions (CDRs) of the amino acid sequence shown in SEQ ID NO: 113; and
(b) a heavy chain variable domain (VH) comprising three complementarity determining regions (CDRs) of the amino acid sequence shown in SEQ ID NO: 112.

In one embodiment, the antigen recognition domain comprises:
(a) a light chain variable domain (VL) comprising three complementarity determining regions (CDRs) of the amino acid sequence shown in SEQ ID NO: 128; and
(b) a heavy chain variable domain (VH) comprising three complementarity determining regions (CDRs) of the amino acid sequence shown in SEQ ID NO: 127.

In one embodiment, the antigen recognition domain comprises:
(a) a light chain variable domain (VL) comprising three complementarity determining regions (CDRs) of the amino acid sequence shown in SEQ ID NO: 142; and
(b) a heavy chain variable domain (VH) comprising three complementarity determining regions (CDRs) of the amino acid sequence shown in SEQ ID NO: 141.

In one embodiment, the antigen recognition domain comprises:
(a) a light chain variable domain (VL) comprising a complementarity determining region (CDR) 1 comprising or consisting of an amino acid sequence of SEQ ID NOs: 29, 30, 44, 45, 59, 60, 75, 76, 95, 96, 107, 108, 122, 123, 137, 138, 151 or 152; a CDR2 comprising or consisting of an amino acid sequence of SEQ ID NOs: 31, 32, 46, 47, 61, 62, 77, 78, 97, 98, 109, 110, 124, 125, 139 or 153; a CDR3 comprising or consisting of an amino acid sequence of SEQ ID NOs: 33, 48, 63, 79, 84, 99, 111, 126, 140 or 154; and
(b) a heavy chain variable domain (VH) comprising a complementarity determining region (CDR) 1 comprising or consisting of an amino acid sequence of SEQ ID NOs: 21, 22, 23, 36, 37, 38, 51, 52, 53, 67, 68, 69, 87, 88, 89, 114, 115, 116, 129, 130, 131, 143, 144 or 145; a CDR2 comprising or consisting of an amino acid sequence of SEQ ID NOs: 24, 25, 26, 39, 40, 41, 54, 55, 56, 70, 71, 72, 90, 91, 92, 102, 103, 104, 117, 118, 119, 132, 133, 134, 146, 147 or 148; a CDR3 comprising or consisting of an amino acid sequence of SEQ ID NOs: 27, 28, 42, 43, 57, 58, 73, 74, 82, 83, 93, 94, 105, 106, 120, 121, 135, 136, 149 or 150.

In one embodiment, the antigen recognition domain comprises:
(a) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 29; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 31; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 33; and
(b) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 21; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 24; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 27, or
(c) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 29; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 31; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 33; and
(d) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 22; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 25; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 27, or
(e) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 30; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 32; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 33; and
(f) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 23; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 26; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 28.

In one embodiment, the antigen recognition domain comprises:
(a) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 44; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 46; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 48; and
(b) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 36; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 39; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 42, or
(c) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 44; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 46; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 48; and
(d) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 37; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 40; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 42, or
(e) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 45; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 47; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 48; and
(f) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 38; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 41; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 43.

In one embodiment, the antigen recognition domain comprises:
(a) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 59; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 61; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 63; and
(b) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 51; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 54; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 57, or
(c) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 59; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 61; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 63; and
(d) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 52; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 55; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 57, or
(e) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 60; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 62; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 63; and
(f) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 53; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 56; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 58.

In one embodiment, the antigen recognition domain comprises:
(a) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 75; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 77; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 79; and
(b) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 67; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 70; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 73, or
(c) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 75; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 77; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 79; and
(d) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 68; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 71; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 73, or
(e) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 76; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 78; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 79; and
(f) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 69; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 72; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 74.

In one embodiment, the antigen recognition domain comprises:
(a) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 75; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 77; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 84; and
(b) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 67; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 70; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 82, or
(c) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 75; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 77; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 84; and
(d) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 68; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 71; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 82, or
(e) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 76; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 78; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 84 and

(f) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 69; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 72; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 83.

In one embodiment, the antigen recognition domain comprises:
(a) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 95; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 97; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 99; and
(b) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 87; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 90; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 93, or
(c) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 95; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 97; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 99; and
(d) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 88; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 91; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 93, or
(e) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 96; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 98; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 99; and
(f) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 89; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 92; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 94.

In one embodiment, the antigen recognition domain comprises:
(a) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 107; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 109; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 111; and
(b) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 21; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 102; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 105, or
(c) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 107; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 109; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 111; and
(d) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 22; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 103; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 105, or
(e) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 108; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 110; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 111; and
(f) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 23; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 104; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 106.

In one embodiment, the antigen recognition domain comprises:
(a) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 122; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 124; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 126; and
(b) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 114; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 117; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 120, or
(c) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 122; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 124; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 126; and
(d) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 115; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 118; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 120, or
(e) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 123; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 125; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 126; and
(f) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 116; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 119; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 121.

In one embodiment, the antigen recognition domain comprises:
(a) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 137; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 139; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 140; and
(b) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 129; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 132; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 135, or
(c) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 137; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 139; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 140; and
(d) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 130; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 133; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 135, or
(e) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 138; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 125; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 140; and
(f) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 131; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 134; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 136.

In one embodiment, the antigen recognition domain comprises:
(a) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 151; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 153; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 154; and
(b) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 143; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 146; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 149, or
(c) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 151; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 153; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 154; and
(d) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 144; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 147; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 149, or
(e) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 152; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 47; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 154; and
(f) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 145; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 148; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 150.

In one embodiment, the antigen recognition domain comprises:
(a) a light chain variable domain (VL) comprising a complementarity determining region (CDR) 1 comprising or consisting of an amino acid sequence of SEQ ID NOs: 29; a CDR2 comprising or consisting of an amino acid sequence of SEQ ID NO: 31; a CDR3 comprising or consisting of an amino acid sequence of SEQ ID NO: 33; and
(b) a heavy chain variable domain (VH) comprising a complementarity determining region (CDR) 1 comprising or consisting of an amino acid sequence of SEQ ID NO: 21; a CDR2 comprising or consisting of an amino acid sequence of SEQ ID NO: 24; a CDR3 comprising or consisting of an amino acid sequence of SEQ ID NO: 27, or
(c) a light chain variable domain (VL) comprising a complementarity determining region (CDR) 1 comprising or consisting of an amino acid sequence of SEQ ID NOs: 29; a CDR2 comprising or consisting of an amino acid sequence of SEQ ID NO: 31; a CDR3 comprising or consisting of an amino acid sequence of SEQ ID NO: 33; and
(d) a heavy chain variable domain (VH) comprising a complementarity determining region (CDR) 1 comprising or consisting of an amino acid sequence of SEQ ID NO: 22; a CDR2 comprising or consisting of an amino acid sequence of SEQ ID NO: 25; a CDR3 comprising or consisting of an amino acid sequence of SEQ ID NO: 27, or
(e) a light chain variable domain (VL) comprising a complementarity determining region (CDR) 1 comprising or consisting of an amino acid sequence of SEQ ID NOs: 30; a CDR2 comprising or consisting of an amino acid sequence of SEQ ID NO: 32; a CDR3 comprising or consisting of an amino acid sequence of SEQ ID NO: 33; and
(f) a heavy chain variable domain (VH) comprising a complementarity determining region (CDR) 1 comprising or consisting of an amino acid sequence of SEQ ID NO: 23; a CDR2 comprising or consisting of an amino acid sequence of SEQ ID NO: 26; a CDR3 comprising or consisting of an amino acid sequence of SEQ ID NO: 28.

In one embodiment, the antigen recognition domain comprises:
(a) a light chain variable domain (VL) comprising a complementarity determining region (CDR) 1 comprising or consisting of an amino acid sequence of SEQ ID NOs: 44; a CDR2 comprising or consisting of an amino acid sequence of SEQ ID NO: 46; a CDR3 comprising or consisting of an amino acid sequence of SEQ ID NO: 48; and
(b) a heavy chain variable domain (VH) comprising a complementarity determining region (CDR) 1 comprising or consisting of an amino acid sequence of SEQ ID NO: 36; a CDR2 comprising or consisting of an amino acid sequence of SEQ ID NO: 39; a CDR3 comprising or consisting of an amino acid sequence of SEQ ID NO: 42, or
(c) a light chain variable domain (VL) comprising a complementarity determining region (CDR) 1 comprising or consisting of an amino acid sequence of SEQ ID NOs: 44; a CDR2 comprising or consisting of an amino acid sequence of SEQ ID NO: 46; a CDR3 comprising or consisting of an amino acid sequence of SEQ ID NO: 48; and
(d) a heavy chain variable domain (VH) comprising a complementarity determining region (CDR) 1 comprising or consisting of an amino acid sequence of SEQ ID NO: 37; a CDR2 comprising or consisting of an amino acid sequence of SEQ ID NO: 40; a CDR3 comprising or consisting of an amino acid sequence of SEQ ID NO: 42, or
(e) a light chain variable domain (VL) comprising a complementarity determining region (CDR) 1 comprising or consisting of an amino acid sequence of SEQ ID NO: 45; a CDR2 comprising or consisting of an amino acid sequence of SEQ ID NO: 47; a CDR3 comprising or consisting of an amino acid sequence of SEQ ID NO: 48; and
(f) a heavy chain variable domain (VH) comprising a complementarity determining region (CDR) 1 comprising or consisting of an amino acid sequence of SEQ ID NO: 38; a CDR2 comprising or consisting of an amino acid sequence of SEQ ID NO: 41; a CDR3 comprising or consisting of an amino acid sequence of SEQ ID NO: 43.

In one embodiment, the antigen recognition domain comprises:
(a) a light chain variable domain (VL) comprising a complementarity determining region (CDR) 1 comprising or consisting of an amino acid sequence of SEQ ID NOs: 59; a CDR2 comprising or consisting of an amino acid sequence of SEQ ID NO: 61; a CDR3 comprising or consisting of an amino acid sequence of SEQ ID NO: 63; and
(b) a heavy chain variable domain (VH) comprising a complementarity determining region (CDR) 1 comprising or consisting of an amino acid sequence of SEQ ID NO: 51; a CDR2 comprising or consisting of an amino acid sequence of SEQ ID NO: 54; a CDR3 comprising or consisting of an amino acid sequence of SEQ ID NO: 57, or
(c) a light chain variable domain (VL) comprising a complementarity determining region (CDR) 1 comprising or consisting of an amino acid sequence of SEQ ID NOs: 59; a CDR2 comprising or consisting of an amino acid sequence of SEQ ID NO: 61; a CDR3 comprising or consisting of an amino acid sequence of SEQ ID NO: 63; and
(d) a heavy chain variable domain (VH) comprising a complementarity determining region (CDR) 1 comprising or consisting of an amino acid sequence of SEQ ID NO: 52; a CDR2 comprising or consisting of an amino acid sequence of SEQ ID NO: 55; a CDR3 comprising or consisting of an amino acid sequence of SEQ ID NO: 57, or
(e) a light chain variable domain (VL) comprising a complementarity determining region (CDR) 1 comprising or consisting of an amino acid sequence of SEQ ID NOs: 60; a CDR2 comprising or consisting of an amino acid sequence of SEQ ID NO: 62; a CDR3 comprising or consisting of an amino acid sequence of SEQ ID NO: 63; and
(f) a heavy chain variable domain (VH) comprising a complementarity determining region (CDR) 1 comprising or consisting of an amino acid sequence of SEQ ID NO: 53; a CDR2 comprising or consisting of an amino acid sequence of SEQ ID NO: 56; a CDR3 comprising or consisting of an amino acid sequence of SEQ ID NO: 58.

In one embodiment, the antigen recognition domain comprises:
(a) a light chain variable domain (VL) comprising a complementarity determining region (CDR) 1 comprising or consisting of an amino acid sequence of SEQ ID NOs: 75; a CDR2 comprising or consisting of an amino acid sequence of SEQ ID NO: 77; a CDR3 comprising or consisting of an amino acid sequence of SEQ ID NO: 79; and
(b) a heavy chain variable domain (VH) comprising a complementarity determining region (CDR) 1 comprising or consisting of an amino acid sequence of SEQ ID NO: 67; a CDR2 comprising or consisting of an amino acid sequence of SEQ ID NO: 70; a CDR3 comprising or consisting of an amino acid sequence of SEQ ID NO: 73, or
(c) a light chain variable domain (VL) comprising a complementarity determining region (CDR) 1 comprising or consisting of an amino acid sequence of SEQ ID NOs: 75; a CDR2 comprising or consisting of an amino acid sequence of SEQ ID NO: 77; a CDR3 comprising or consisting of an amino acid sequence of SEQ ID NO: 79; and
(d) a heavy chain variable domain (VH) comprising a complementarity determining region (CDR) 1 comprising or consisting of an amino acid sequence of SEQ ID NO: 68; a CDR2 comprising or consisting of an amino acid sequence of SEQ ID NO: 71; a CDR3 comprising or consisting of an amino acid sequence of SEQ ID NO: 73, or
(e) a light chain variable domain (VL) comprising a complementarity determining region (CDR) 1 comprising or consisting of an amino acid sequence of SEQ ID NOs: 76; a CDR2 comprising or consisting of an amino acid sequence of SEQ ID NO: 78; a CDR3 comprising or consisting of an amino acid sequence of SEQ ID NO: 79; and
(f) a heavy chain variable domain (VH) comprising a complementarity determining region (CDR) 1 comprising or consisting of an amino acid sequence of SEQ ID NO: 69; a CDR2 comprising or consisting of an amino acid sequence of SEQ ID NO: 72; a CDR3 comprising or consisting of an amino acid sequence of SEQ ID NO: 74.

In one embodiment, the antigen recognition domain comprises:
(a) a light chain variable domain (VL) comprising a complementarity determining region (CDR) 1 comprising or consisting of an amino acid sequence of SEQ ID NOs: 75; a CDR2 comprising or consisting of an amino acid sequence of SEQ ID NO: 77; a CDR3 comprising or consisting of an amino acid sequence of SEQ ID NO: 84; and
(b) a heavy chain variable domain (VH) comprising a complementarity determining region (CDR) 1 comprising or consisting of an amino acid sequence of SEQ ID NO: 67; a CDR2 comprising or consisting of an amino acid sequence of SEQ ID NO: 70; a CDR3 comprising or consisting of an amino acid sequence of SEQ ID NO: 82, or
(c) a light chain variable domain (VL) comprising a complementarity determining region (CDR) 1 comprising or consisting of an amino acid sequence of SEQ ID NOs: 75; a CDR2 comprising or consisting of an amino acid sequence of SEQ ID NO: 77; a CDR3 comprising or consisting of an amino acid sequence of SEQ ID NO: 84; and
(d) a heavy chain variable domain (VH) comprising a complementarity determining region (CDR) 1 comprising or consisting of an amino acid sequence of SEQ ID NO: 68; a CDR2 comprising or consisting of an amino acid sequence of SEQ ID NO: 71; a CDR3 comprising or consisting of an amino acid sequence of SEQ ID NO: 82, or
(e) a light chain variable domain (VL) comprising a complementarity determining region (CDR) 1 comprising or consisting of an amino acid sequence of SEQ ID NOs: 76; a CDR2 comprising or consisting of an amino acid sequence of SEQ ID NO: 78; a CDR3 comprising or consisting of an amino acid sequence of SEQ ID NO: 84; and
(f) a heavy chain variable domain (VH) comprising a complementarity determining region (CDR) 1 comprising or consisting of an amino acid sequence of SEQ ID NO: 69; a CDR2 comprising or consisting of an amino acid sequence of SEQ ID NO: 72; a CDR3 comprising or consisting of an amino acid sequence of SEQ ID NO: 83.

In one embodiment, the antigen recognition domain comprises:
(a) a light chain variable domain (VL) comprising a complementarity determining region (CDR) 1 comprising or consisting of an amino acid sequence of SEQ ID NOs: 95; a CDR2 comprising or consisting of an amino acid sequence of SEQ ID NO: 97; a CDR3 comprising or consisting of an amino acid sequence of SEQ ID NO: 99; and
(b) a heavy chain variable domain (VH) comprising a complementarity determining region (CDR) 1 comprising or consisting of an amino acid sequence of SEQ ID NO: 87; a CDR2 comprising or consisting of an amino acid sequence of SEQ ID NO: 90; a CDR3 comprising or consisting of an amino acid sequence of SEQ ID NO: 93, or
(c) a light chain variable domain (VL) comprising a complementarity determining region (CDR) 1 comprising or consisting of an amino acid sequence of SEQ ID NOs: 95; a CDR2 comprising or consisting of an amino acid sequence of SEQ ID NO: 97; a CDR3 comprising or consisting of an amino acid sequence of SEQ ID NO: 99; and
(d) a heavy chain variable domain (VH) comprising a complementarity determining region (CDR) 1 comprising or consisting of an amino acid sequence of SEQ ID NO: 88; a CDR2 comprising or consisting of an amino acid sequence of SEQ ID NO: 91; a CDR3 comprising or consisting of an amino acid sequence of SEQ ID NO: 93, or
(e) a light chain variable domain (VL) comprising a complementarity determining region (CDR) 1 comprising or consisting of an amino acid sequence of SEQ ID NOs: 96; a CDR2 comprising or consisting of an amino acid sequence of SEQ ID NO: 98; a CDR3 comprising or consisting of an amino acid sequence of SEQ ID NO: 99; and
(f) a heavy chain variable domain (VH) comprising a complementarity determining region (CDR) 1 comprising or consisting of an amino acid sequence of SEQ ID NO: 89; a CDR2 comprising or consisting of an amino acid sequence of SEQ ID NO: 92; a CDR3 comprising or consisting of an amino acid sequence of SEQ ID NO: 94.

In one embodiment, the antigen recognition domain comprises:
(a) a light chain variable domain (VL) comprising a complementarity determining region (CDR) 1 comprising or consisting of an amino acid sequence of SEQ ID NOs: 107; a CDR2 comprising or consisting of an amino acid sequence of SEQ ID NO: 109; a CDR3 comprising or consisting of an amino acid sequence of SEQ ID NO: 111; and
(b) a heavy chain variable domain (VH) comprising a complementarity determining region (CDR) 1 comprising or consisting of an amino acid sequence of SEQ ID NO: 21; a CDR2 comprising or consisting of an amino acid sequence of SEQ ID NO: 102; a CDR3 comprising or consisting of an amino acid sequence of SEQ ID NO: 105, or
(c) a light chain variable domain (VL) comprising a complementarity determining region (CDR) 1 comprising or consisting of an amino acid sequence of SEQ ID NOs: 107; a CDR2 comprising or consisting of an amino acid sequence of SEQ ID NO: 109; a CDR3 comprising or consisting of an amino acid sequence of SEQ ID NO: 111; and
(d) a heavy chain variable domain (VH) comprising a complementarity determining region (CDR) 1 comprising or consisting of an amino acid sequence of SEQ ID NO: 22; a CDR2 comprising or consisting of an amino acid sequence of SEQ ID NO: 103; a CDR3 comprising or consisting of an amino acid sequence of SEQ ID NO: 105, or
(e) a light chain variable domain (VL) comprising a complementarity determining region (CDR) 1 comprising or consisting of an amino acid sequence of SEQ ID NOs: 108; a CDR2 comprising or consisting of an amino acid sequence of SEQ ID NO: 110; a CDR3 comprising or consisting of an amino acid sequence of SEQ ID NO: 111; and
(f) a heavy chain variable domain (VH) comprising a complementarity determining region (CDR) 1 comprising or consisting of an amino acid sequence of SEQ ID NO: 23; a CDR2 comprising or consisting of an amino acid sequence of SEQ ID NO: 104; a CDR3 comprising or consisting of an amino acid sequence of SEQ ID NO: 106.

In one embodiment, the antigen recognition domain comprises:
(a) a light chain variable domain (VL) comprising a complementarity determining region (CDR) 1 comprising or consisting of an amino acid sequence of SEQ ID NOs: 122; a CDR2 comprising or consisting of an amino acid sequence of SEQ ID NO: 124; a CDR3 comprising or consisting of an amino acid sequence of SEQ ID NO: 126; and
(b) a heavy chain variable domain (VH) comprising a complementarity determining region (CDR) 1 comprising or consisting of an amino acid sequence of SEQ ID NO: 114; a CDR2 comprising or consisting of an amino acid sequence of SEQ ID NO: 117; a CDR3 comprising or consisting of an amino acid sequence of SEQ ID NO: 120, or
(c) a light chain variable domain (VL) comprising a complementarity determining region (CDR) 1 comprising or consisting of an amino acid sequence of SEQ ID NOs: 122; a CDR2 comprising or consisting of an amino acid sequence of SEQ ID NO: 124; a CDR3 comprising or consisting of an amino acid sequence of SEQ ID NO: 126; and
(d) a heavy chain variable domain (VH) comprising a complementarity determining region (CDR) 1 comprising or consisting of an amino acid sequence of SEQ ID NO: 115; a CDR2 comprising or consisting of an amino acid sequence of SEQ ID NO: 118; a CDR3 comprising or consisting of an amino acid sequence of SEQ ID NO: 120, or
(e) a light chain variable domain (VL) comprising a complementarity determining region (CDR) 1 comprising or consisting of an amino acid sequence of SEQ ID NOs: 123; a CDR2 comprising or consisting of an amino acid sequence of SEQ ID NO: 125; a CDR3 comprising or consisting of an amino acid sequence of SEQ ID NO: 126; and
(f) a heavy chain variable domain (VH) comprising a complementarity determining region (CDR) 1 comprising or consisting of an amino acid sequence of SEQ ID NO: 116; a CDR2 comprising or consisting of an amino acid sequence of SEQ ID NO: 119; a CDR3 comprising or consisting of an amino acid sequence of SEQ ID NO: 121.

In one embodiment, the antigen recognition domain comprises:
(a) a light chain variable domain (VL) comprising a complementarity determining region (CDR) 1 comprising or consisting of an amino acid sequence of SEQ ID NOs: 137; a CDR2 comprising or consisting of an amino acid sequence of SEQ ID NO: 139; a CDR3 comprising or consisting of an amino acid sequence of SEQ ID NO: 140; and
(b) a heavy chain variable domain (VH) comprising a complementarity determining region (CDR) 1 comprising or consisting of an amino acid sequence of SEQ ID NO: 129; a CDR2 comprising or consisting of an amino acid sequence of SEQ ID NO: 132; a CDR3 comprising or consisting of an amino acid sequence of SEQ ID NO: 135, or
(c) a light chain variable domain (VL) comprising a complementarity determining region (CDR) 1 comprising or consisting of an amino acid sequence of SEQ ID NOs: 137; a CDR2 comprising or consisting of an amino acid sequence of SEQ ID NO: 139; a CDR3 comprising or consisting of an amino acid sequence of SEQ ID NO: 140; and
(d) a heavy chain variable domain (VH) comprising a complementarity determining region (CDR) 1 comprising or consisting of an amino acid sequence of SEQ ID NO: 130; a CDR2 comprising or consisting of an amino acid sequence of SEQ ID NO: 133; a CDR3 comprising or consisting of an amino acid sequence of SEQ ID NO: 135, or
(e) a light chain variable domain (VL) comprising a complementarity determining region (CDR) 1 comprising or consisting of an amino acid sequence of SEQ ID NOs: 138; a CDR2 comprising or consisting of an amino acid sequence of SEQ ID NO: 125; a CDR3 comprising or consisting of an amino acid sequence of SEQ ID NO: 140; and
(f) a heavy chain variable domain (VH) comprising a complementarity determining region (CDR) 1 comprising or consisting of an amino acid sequence of SEQ ID NO: 131; a CDR2 comprising or consisting of an amino acid sequence of SEQ ID NO: 134; a CDR3 comprising or consisting of an amino acid sequence of SEQ ID NO: 136.

In one embodiment, the antigen recognition domain comprises:
(a) a light chain variable domain (VL) comprising a complementarity determining region (CDR) 1 comprising or consisting of an amino acid sequence of SEQ ID NOs: 151; a CDR2 comprising or consisting of an amino acid sequence of SEQ ID NO: 153; a CDR3 comprising or consisting of an amino acid sequence of SEQ ID NO: 154; and
(b) a heavy chain variable domain (VH) comprising a complementarity determining region (CDR) 1 comprising or consisting of an amino acid sequence of SEQ ID NO: 143; a CDR2 comprising or consisting of an amino acid sequence of SEQ ID NO: 146; a CDR3 comprising or consisting of an amino acid sequence of SEQ ID NO: 149, or
(c) a light chain variable domain (VL) comprising a complementarity determining region (CDR) 1 comprising or consisting of an amino acid sequence of SEQ ID NOs: 151; a CDR2 comprising or consisting of an amino acid sequence of SEQ ID NO: 153; a CDR3 comprising or consisting of an amino acid sequence of SEQ ID NO: 154; and
(d) a heavy chain variable domain (VH) comprising a complementarity determining region (CDR) 1 comprising or consisting of an amino acid sequence of SEQ ID NO: 144; a CDR2 comprising or consisting of an amino acid sequence of SEQ ID NO: 147; a CDR3 comprising or consisting of an amino acid sequence of SEQ ID NO: 149, or
(e) a light chain variable domain (VL) comprising a complementarity determining region (CDR) 1 comprising or consisting of an amino acid sequence of SEQ ID NOs: 152; a CDR2 comprising or consisting of an amino acid sequence of SEQ ID NO: 47; a CDR3 comprising or consisting of an amino acid sequence of SEQ ID NO: 154; and
(f) a heavy chain variable domain (VH) comprising a complementarity determining region (CDR) 1 comprising or consisting of an amino acid sequence of SEQ ID NO: 145; a CDR2 comprising or consisting of an amino acid sequence of SEQ ID NO: 148; a CDR3 comprising or consisting of an amino acid sequence of SEQ ID NO: 150.

In certain embodiments, the antigen binding domain comprises or consists of an amino acid sequence having at least 75%, 80%, 85%, 90%, 91%, 92%, 93%, 94%, 95%, 96%, 97%, 98%, 99%, or any percentage in between of identity to the amino acid sequence as set forth in SEQ ID NO: 1.

In certain embodiments, the antigen binding domain comprises or consists of an amino acid sequence as set forth in SEQ ID NO: 1 or SEQ ID NO: 166 with 0-10 amino acid insertions, deletions, substitutions, additions, or a combination thereof, at one or more positions other than the CDR regions.

In certain embodiments, the antigen binding domain comprises or consists of an amino acid sequence having at least 75%, 80%, 85%, 90%, 91%, 92%, 93%, 94%, 95%, 96%, 97%, 98%, 99%, or any percentage in between of identity to the amino acid sequence as set forth in SEQ ID NO: 3.

In certain embodiments, the antigen binding domain comprises or consists of an amino acid sequence as set forth in SEQ ID NO: 3 with 0-10 amino acid insertions, deletions, substitutions, additions, or a combination thereof, at one or more positions other than the CDR regions.

In certain embodiments, the antigen binding domain comprises or consists of an amino acid sequence having at least 75%, 80%, 85%, 90%, 91%, 92%, 93%, 94%, 95%, 96%, 97%, 98%, 99%, or any percentage in between of identity to the amino acid sequence as set forth in SEQ ID NO: 5.

In certain embodiments, the antigen binding domain comprises or consists of an amino acid sequence as set forth in SEQ ID NO: 5 with 0-10 amino acid insertions, deletions, substitutions, additions, or a combination thereof, at one or more positions other than the CDR regions.

In a more specific example, the antigen binding domain comprises or consists of the amino acid sequence as set forth in SEQ ID: 1; comprises or consists of amino acid sequence as set forth in SEQ ID NO: 3; or comprises or consists of the amino acid sequence as set forth in SEQ ID NO: 5.

In a more specific example, the scFv of i) has the amino acid sequence as set forth in SEQ ID NO: 1; the scFv of ii) has the amino acid sequence as set forth in SEQ ID NO: 3; and the scFv of iii) has the amino acid sequence as set forth in SEQ ID NO: 5.

In another aspect, the present disclosure refers to a nucleic acid comprising a nucleotide sequence encoding the chimeric antigen receptor as disclosed herein. A nucleic acid encoding chimeric antigen receptor as disclosed herein may also be referred to as a first nucleic acid (in the context where the disclosure refers to a second and/or third nucleic acid).

In another aspect, the present disclosure refers to a first polynucleotide encoding the chimeric antigen receptor as disclosed herein.

In another aspect, the present disclosure also refers to a nucleic acid construct or vector comprising the first nucleic acid or first polynucleotide as disclosed herein. In some embodiments, expression of the nucleic acid molecule is under the control of a transcriptional control sequence. In some embodiments, the transcriptional control sequence may be a constitutive promoter or an inducible promoter. In some embodiments, the vector is as a viral vector, which can be used to transform an immune cell, such as a T cell, to induce expression of the CAR.

In another aspect of the present disclosure, there is provided a genetically modified cell that includes a CAR as disclosed herein.

In another aspect of the present disclosure, there is provided a genetically modified cell that includes a nucleic acid molecule as described herein, or a nucleic acid construct or vector as described herein, or a genomically integrated form of the construct or vector. The genetically modified cell may be a T cell, for example a Treg, an iPSC-derived T cell, for example an iPSC-derived Treg cell, or a CD34+ iPSC.

In another aspect of the present disclosure, there is provided a modified T cell comprising the chimeric antigen receptor as disclosed herein, the first polynucleotide or first nucleic acid as disclosed herein, and/or the nucleic acid construct or vector as disclosed herein. The modified T cell may be a Treg cell, for example an iPSC-derived Treg cell.

In one embodiment, the genetically modified cell, preferably T cell, is further modified to express one or more immunosuppressive molecules. The expression can be constitutive expression or inducible expression. Preferably, the one or more immunosuppressive molecules are expressed when the modified T cell is activated, for example when the T cell is activated by the chimeric antigen receptor disclosed herein and expressed by the modified T cell, specifically through its signalling domain.

In one embodiment, the inducible expression is enabled by a NFAT-inducible system. Preferably, the modified T cell comprises a second nucleic acid or polynucleotide encoding a promoter comprising one or more binding sites for NFAT (nuclear factor of activated T cells), and one or more immunosuppressive molecules; wherein the binding of NFAT to the promoter induces the expression of the one or more immune-suppressive molecules.

In one example, both the first and the second nucleic acids or polynucleotide, as disclosed herein, are expressed from one nucleic acid expression sequence.

Exemplary immunosuppressors or immune-suppressive molecules are, but are not limited to, IL-10, TGF-beta, FOXP3 and combinations thereof. In an example, the one or more immunosuppressive molecules are capable of suppressing the activity of effecter T cells or are capable of inducing anergy in effector T cells. In a specific example, the one or more immunosuppressive molecules are selected from the group consisting of IL-10, TGF-beta, CTLA-4, LAG3, PD-L1 and PD-1. In a specific example, the one or more immunosuppressive molecules is/are IL-10 and/or TGF-beta. In a specific example, the second polynucleotide comprises the sequence as set forth in SEQ ID: 10.

In another aspect of the present disclosure, there is provided a pharmaceutical composition comprising a genetically modified cell as disclosed herein and a pharmaceutically acceptable carrier, diluent or excipient.

In another aspect of the present disclosure, there is provided a method of generating Chimeric antigen receptor (CAR)-regulatory T cells (CAR-Tregs), said method comprising transducing Tregs with the vector as disclosed herein, so that the transduced Tregs express the CAR of the present disclosure, thereby generating Chimeric antigen receptor (CAR)-regulatory T cells (CAR-Tregs).

In another aspect of the present disclosure, there is provided a method of generating Chimeric antigen receptor (CAR)-regulatory T cells (CAR-Tregs), said method comprising: a) isolating regulatory T cells (Treg) from peripheral blood mononuclear cells (PBMCs) from a subject; b) expanding the isolated Tregs ex vivo; and c) transducing the expanded Tregs with the vector as disclosed herein, so that the transduced Tregs express the CAR of the present disclosure.

In another aspect of the present disclosure, there is provided a method of generating modified a hypoimmunogenic T cell, said method comprising: a) modifying a CD34+ iPSC to (i) reduce or eliminate the level of expression or activity of B2 microglobulin and class II MHC transactivator, and (ii) overexpress CD47 or a functional variant thereof; b) transducing the modified CD34+ iPSC with a vector of the present disclosure, so that the transduced CD34+ iPSC expresses the CAR of the present disclosure; c) differentiating the CAR hypoimmunogenic iPSC to an iPSC-derived T cell. Preferably the iPSC-derived T cell is an iPSC-derived T-regulatory (Treg) cell.

In another aspect of the present disclosure, there is provided a method of generating modified a hypoimmunogenic T cell, said method comprising: a) providing a CD34+ iPSC, b) reducing or eliminating the level of expression of activity of B2 microglobulin and class II MHC transactivator in the CD34+ iPSC; c) overexpressing CD47 or a functional variant thereof in the CD34+ iPSC of b); d) transducing the CD34+ iPSC of c) with a vector of the present disclosure, so that the transduced CD34+ iPSC expresses the CAR of the present disclosure; e) differentiating the CAR hypoimmunogenic iPSC to an iPSC-derived T cell. Preferably the iPSC-derived T cell is an iPSC-derived T-regulatory (Treg) cell.

In another aspect of the present disclosure, there is provided a method of generating modified hypoimmunogenic T cells, said method comprising: a) editing the genome of CD34+ iPSCs to reduce or eliminate the expression of functional gene products of B2M (B2 microglobulin) and CIITA (class II MHC transactivator) genes; c) over expressing CD47 or a functional variant thereof in the CD34+ iPSCs; d) transducing the CD34+ iPSCs of c) with the vector of the present disclosure, so that the transduced cells express the CAR of the present disclosure; e) differentiating the CAR hypoimmunogenic iPSCs to iPSC-derived T cells. Preferably the iPSC-derived T cells are iPSC-derived T-regulatory (Treg) cells.

In another aspect of the present disclosure, there is provided a method of generating modified hypoimmunogenic T cells, said method comprising: a) CD34+ induced pluripotent stem cells; b) editing the genome of the CD34+ iPSCs to knock out both B2M (B2 microglobulin) and CIITA (class II MHC transactivator) genes; c) genetically incorporating CD47 or a functional variant thereof, wherein the transduced iPSC overexpresses CD47; d) transducing the T cells of e) with the vector of the present disclosure, so that the transduced T cells express the CAR of the present disclosure; f) differentiating the CAR hypoimmunogenic iPSCs to iPSC-derived T cells.

In another aspect, the present invention refers to a method of generating chimeric antigen receptor (CAR)-regulatory T cells (CAR-Tregs), said method comprising isolating regulatory T cells (Treg) from peripheral blood mononuclear cells (PBMCs) from a subject; expanding the isolated Tregs ex vivo; and transducing the expanded Tregs with the vector as disclosed herein, so that the transduced Tregs express the CAR as disclosed herein.

In yet another aspect, the present disclosure refers to a genetically modified cell or a modified T cell of the present disclosure for use in therapy. Preferably the modified T cell is a modified Treg cell expressing a CAR of the present disclosure. In one embodiment, the Treg cell may be an iPSC-derived Treg cell.

In yet another aspect, the present disclosure refers to a genetically modified cell or modified T cell of the present disclosure for use in the treatment of an autoimmune disease; a transplant rejection, a graft versus host disease (GVHD), a cytokine release syndrome or any disease/condition involving or resulting from uncontrolled inflammatory responses mediated by one or more inflammation associated factor(s). Preferably the modified T cell is a modified Treg cell expressing a CAR of the present disclosure. In one embodiment, the Treg cell may be an iPSC-derived Treg cell.

In yet another aspect, the present disclosure refers to a method of treating an autoimmune disease, a transplant rejection, a graft versus host disease (GVHD), a cytokine release syndrome or any disease/condition involving or resulting from uncontrolled inflammatory responses mediated by one or more inflammation associated factor(s), wherein the method comprises administering a therapeutically effective number of the genetically modified cells or the modified T cells of the present disclosure, thereby treating an autoimmune disease, a transplant rejection, or a graft versus host disease (GVHD).

In still another aspect, the present disclosure refers to a method of inducing immune tolerance in a subject in need thereof, wherein the method comprises administering a therapeutically effective number of the modified T cells as disclosed herein, thereby inducing immune tolerance in the subject in need thereof.

In still another aspect, the present disclosure refers to a method of downregulating inflammation locally or systemically in a subject in need thereof, wherein the method comprises administering a therapeutically effective number of the modified T cells as disclosed herein, thereby downregulating inflammation locally or systemically in a subject in need thereof.

In still another aspect, the present disclosure refers to a method of suppressing the activity of effector T cells locally or systematically in a subject, wherein the method comprises administering a modified T cells as disclosed herein, or a therapeutically effective number of modified T cells as disclosed herein of the present disclosure to the subject locally or systematically, thereby suppressing the activity of effector T cells locally or systematically in the subject.

In still another aspect, the present disclosure provides use of a nucleic acid, a nucleic acid construct, or a modified cell as described herein in the manufacture of a medicament for:
treating an autoimmune disease, a transplant rejection, a graft versus host disease (GVHD), a cytokine release syndrome or any disease/condition involving or resulting from uncontrolled inflammatory responses mediated by one or more inflammation associated factor(s);
inducing immune tolerance in a subject in need thereof,
downregulating inflammation locally or systemically in a subject in need thereof, or
suppressing the activity of effector T cells locally or systematically in a subject.

In another aspect of the present disclosure, there is provided a vector comprising a sequence as provided in the present disclosure.

### BRIEF DESCRIPTION OF THE DRAWINGS

The invention will be better understood with reference to the detailed description when considered in conjunction with the non-limiting examples and the accompanying drawings, in which:

FIG. 1 shows a schematic drawing of an exemplary design of the chimeric antigen receptor which binds pro-inflammatory cytokines, and an inducible expression cassette encoding immunosuppressor molecules. In this example, TNF-alpha and/or IFN-gamma are the pro-inflammatory cytokines to be targeted by the antigen binding domain(s) of the chimeric antigen receptor. Also in this example, the immunosuppressor molecules are IL-10 and TGF-beta.

FIG. 2 shows a schematic map of polynucleotide constructs encoding exemplary chimeric antigen receptors (CAR) targeting, for example, tumour necrosis factor alpha (TNF-alpha; TNFa), and further encoding a Nuclear factor of activated T cells (NFAT) inducible expression cassette. (A) In this example, the chimeric antigen receptor construct PMC882 comprises a single chain variable fragment (scFv), which binds the pro-inflammatory cytokine T^{N}F-alpha, and comprises a CD28 co-stimulatory domain, as well as a CD3-zeta signalling domain. In this example, the chimeric antigen receptor coding sequence is preceded by a CD8 leader sequence, which is preceded by a MNDU3 promoter. In this specific example, FIG. 2A, the construct also encodes a NFAT inducible expression cassette, which is found downstream of CAR cassette. The NFAT inducible expression cassette encodes a 6(NFAT)minimal IL-2 promoter, followed by the coding sequences of TGF-beta and IL-10, two immunosuppressor molecules exemplified in this example. A self-cleavable T2A peptide is also encoded between the two immunosuppressors. (B) In another example, FOXP3, another example of an immunosuppressor molecule, is co-expressed with the CAR cassette and is separated from CD3-zeta signalling domain by the self-cleavable T2A peptide.

FIG. 3 shows the exemplary sorting of a healthy donor peripheral blood mononuclear cells (PBMCs) using fluorescence-activated cell sorting (FACS), as well as the purity of the sorted populations. (A) Peripheral blood mononuclear cells (PBMCs) were labelled with anti-CD4, CD4, CD25, CD127 surface markers and sorted as shown. (B) This sorting strategy yielded high purity of CD4 CD25, CD127-T_{reg} based on FOXP3 expression (top panel) as compared to CD4⁺, CD25⁻, CD127⁺T effector cells (bottom panel). T_{reg} isolated using fluorescence-activated cell sorting (FACS) were subsequently transduced and expanded to generate CAR T_{regs}.

FIG. 4 shows results of expansion, as well as expression, of transduced CAR Tregs. (A) is a graph showing fold expansion of healthy Tregs in the production of CAR Tregs over 14 days. The results indicate a robust expansion of over 150-fold. (B) GFP CAR Tregs shows a transduction of 75% in expanded Tregs compared to untransduced expanded Tregs.

FIG. 5 shows the results of Jurkat cells transduced with CAR PMC882. The graph shows an increase in surface expression of the activation marker, CD69, determined by flow cytomery of CD69+ cells, over untransduced Jurkat. This increased expression demonstrates activation due to the presence of TNFa. Error bars indicating standard deviation; *p<0.05.

FIG. 6 shows the results of flow cytometry analysis based on intracellular staining of TGF beta and IL10 in transduced (A) Jurkat cells, (B) healthy Tregs and (C) active disease Tregs, both in the presence or absence of TNFa. This was done to simulate and demonstrate inducible expression by the NFAT promotor. (A) Jurkat cells transduced with CAR PMC882 demonstrated increased expression of immunosuppressive TGFb and IL0 in the presence of TNTa, as compared no TNFa stimulation. (B) CAR PMC882 Tregs from healthy donor and (C) donor with active disease (juvenile idiopathic arthritis; JIA) showed increased expression of TGFb and IL10 in the presence of TNFa, as compared to no TNFa stimulation. These results illustrate the inducible expression of TGFb and IL10 using the NFAT promotor in PMC882. Error bars indicating standard deviation; *p<0.05.

FIG. 7 shows data indicating a reduction of inflammatory T cells by co-culture with CAR Tregs PMC882 from (A) healthy donor and (B) active disease donor (JIA) compared to untransduced Tregs in the presence of CD3/28 activators during a 24-hour co-culture. Error bars indicating standard deviation; *p<0.05.

FIG. 8 shows suppression of proliferating effector T cells that were activated by CD3/28 activators by PMC882 transduced CAR Tregs over 7 days. (A) Healthy donor Tregs, as well as (B) active JIA disease Tregs, were transduced with CAR PMC882. The results show a suppression of proliferating T cells. Error bars indicating standard deviation; *p<0.05; **p<0.005.

FIG. 9 shows the effect of PMC882 CAR T_{reg} treatment in the NSG GvHD mouse model. (A) The clinical GvHD score of PMC882 CAR T_{reg} treated mice compared to untreated mice. Error bars indicating standard deviation; Area under the curve *p<0.0001; n=6 per group. (B) Survival curve of PMC882 CAR T_{reg} treated mice compared to untreated mice. Logrank test *p<0.05. (C) The percent engraftment of human CD45 in the peripheral blood is suppressed in the PMC882 CAR T_{reg} treated mice compared to untreated at Day 14. Error bars indicating standard deviation; Student's t-test **p<0.005; n=6. (D) The percent of human CD45 engraftment at the end of the experiment at Day 35, PMC882 CAR T_{reg} treated mice showed significant suppression of human CD45 engraftment compared to untreated mice. Student's t-test **p<0.005; n=4. (E) Liver tissue analysis using VECTRA that stains for CD4⁻ (yellow) and CD8′ (red) indicating the T cell infiltrate in the tissue. (F) High dimensional reduction tSNE analysis of CD3⁺ T cells in the peripheral blood using CyToF (top panel) showing well separated clusters from CAR T_{reg} treated mice (middle panel) and untreated mice (bottom panel). (G) Heatmap of markers which make up the clusters from CAR T_{reg} treated mice (left) and untreated mice (right).

FIG. 10 shows anti-IFNy CAR Jurkat (A) and healthy donor Tregs (B) are responsive to soluble human IFNy and induces TGFb (left two columns in each of (A) and (B)) and IL10 (right two columns in each of (A) and (B)) upregulation. Error bars indicating standard deviation; Student's t-test *p<0.05. Data from 3 independent experiments.

FIG. 11 demonstrates that both anti-TNFa CAR PMC882 and anti-IFNy CAR Tregs are able to suppress effector T cells from proliferating significantly better than untransduced Tregs in healthy donors. Error bars indicating standard error of the mean; Two-way ANOVA, Tukey's multiple comparison test; *p<0.05; **p<0.005. Data from 3 healthy donors and from 3 independent experiments.

FIG. 12 shows that the genetically engineered iPS cells demonstrates knockout of HLA A, B, C (A) and HLA DR, DP, DQ (B) as well as the overexpression of CD47 (C) by surface staining with flow cytometry compared to wildtype iPS cells. (D) shows that the engineered iPS do not induce T cell proliferation compared to wildtype iPS cells. Error bars indicating standard error of the mean; One-way ANOVA, Tukey's multiple comparison test; ***p<0.0005. Data from 3 healthy donors

FIG. 13 details the differentiation of iPS cells to CD34+ cells by Day 12 (A) and subsequent differentiation to T cells showing expression of CD3, CD4 and CD8 (B). The iPS-derived T cells transduced with anti-TNFa CAR with GFP under the inducible NFAT promotor and upon PMA stimulation, show upregulation of GFP expression.

### DESCRIPTION OF SEQUENCES

TABLE 1
Sequences of the invention

| SEQ | | |
|---|---|---|
| ID NO | Descriptor | Sequence |
| 1 | Exemplary amino acid | EVQLLESGGGLVQPGGSLRLSCAASGFTFSSYAMSWVRQAPG |
| | sequence of a TNF-alpha | KGLEWVSSISSTGASTTYADSVKGRFTISRDNSKNTLYLQMN |
| | scFv clone P3H2 | SLRAEDTAVYYCAKGGAAFDYWGQGTLVTVSSGGGGSGGGGS |
| | | GGGGSDIQMTQSPSSLSASVGDRVTITCRASQSISSYLNWYQ |
| | | QKPGKAPKLLIYSASYLQSGVPSRFSGSGSGTDFTLTISSLQ |
| | | PEDFATYYCQQANNAPTTFGQGTKVEIK |
| 2 | An exemplary coding | GAGGTGCAGCTGTTGGAGTCTGGGGGAGGCTTGGTACAGCCT |
| | sequence for the TNF- | GGGGGGTCCCTGAGACTCTCCTGTGCAGCCTCTGGATTCACC |
| | alpha scFv clone P3H2 | TTTAGCAGCTATGCCATGAGCTGGGTCCGCCAGGCTCCAGGG |
| | | AAGGGGCTGGAGTGGGTCTCAAGTATTTCTTCTACTGGTGCT |
| | | TCTACAACTTACGCAGACTCCGTGAAGGGCAGGTTCACCATC |
| | | TCCAGAGACAATTCCAAGAACACGCTGTATCTGCAAATGAAC |
| | | AGCCTGAGAGCCGAGGACACGGCCGTATATTACTGTGCGAAA |
| | | GGTGGTGCTGCTTTTGACTACTGGGGCCAGGGAACCCTGGTC |
| | | ACCGTCAGCAGCGGTGGAGGTGGTTCTGGTGGAGGAGGTTCT |
| | | GGTGGCGGTGGTTCTGACATCCAGATGACCCAGTCTCCATCC |
| | | TCCCTGTCTGCATCTGTAGGAGACAGAGTCACCATCACTTGC |
| | | CGGGCAAGTCAGAGCATTAGCAGCTATTTAAATTGGTATCAG |
| | | CAGAAACCAGGGAAAGCCCCTAAGCTCCTGATCTATAGTGCA |
| | | TCCTATTTGCAAAGTGGGGTCCCATCAAGGTTCAGTGGCAGT |
| | | GGATCTGGGACAGATTTCACTCTCACCATCAGCAGTCTGCAA |
| | | CCTGAAGATTTTGCAACTTACTACTGTCAACAGGCTAATAAT |
| | | GCTCCTACTACGTTCGGCCAAGGGACCAAGGTGGAAATCAAA |
| 3 | Exemplary amino acid | EVKLEQSGTVLARPGASVKMSCKASGYTFTNYWMHWIRQRPG |
| | sequence of an IFN- | QGLEWIGAIYPGISDTSYNQKFKVKAKLTAVTSTSTAYMELS |
| | gamma scFv TII 6AE52 | SLTNEDSAVYYCTRDEDFDGFAYWGQGTLVTVSAGGGGSGGG |
| | | GSGGGGSDVLMTQTPLSLPVSLGDQASISCRSSQSIVHSNGN |
| | | TYLDWYLQKPGQSPKLLIYKVSNRFSGVPDRFSGSGSGTDFT |
| | | LRISRVEAEDLGVYYCFRGSHAPYTFGGGTKLEIKR |
| 4 | An exemplary coding | GAGGTCAAGCTGGAGCAGTCTGGGACTGTGCTGGCAAGGCCT |
| | sequence for the IFN- | GGGGCTTCAGTGAAGATGTCCTGCAAGGCTTCTGGCTACACC |
| | gamma scFv TII 6AE52 | TTTACCAACTACTGGATGCATTGGATAAGACAGAGGCCTGGA |
| | | CAGGGTCTGGAATGGATTGGCGCTATTTATCCTGGAATTAGT |
| | | GATACTAGTTACAACCAGAAGTTCAAGGTCAAGGCCAAACTG |
| | | ACTGCAGTCACATCCACCAGCACTGCCTACATGGAGCTCAGC |
| | | AGCCTGACAAATGAGGACTCTGCGGTCTATTATTGTACAAGA |
| | | GATGAGGATTTCGACGGCTTTGCTTACTGGGGCCAAGGGACT |
| | | CTGGTCACTGTCTCTGCAGGTGGTGGTGGGTCTGGCGGAGGT |
| | | GGTAGTGGAGGTGGTGGGTCTGATGTTTTGATGACCCAAACT |
| | | CCACTCTCCCTGCCTGTCAGTCTTGGAGATCAAGCCTCCATC |
| | | TCTTGCAGATCTAGTCAGAGTATTGTACATAGTAATGGAAAC |
| | | ACCTATTTAGACTGGTACCTGCAGAAACCAGGCCAGTCTCCA |
| | | AAGCTCCTGATCTACAAAGTTTCCAACCGATTTTCTGGGGTC |
| | | CCAGACAGGTTCAGTGGCAGTGGATCAGGGACAGATTTCACA |
| | | CTCAGGATCAGCAGAGTGGAGGCTGAGGATCTGGGAGTTTAT |
| | | TACTGCTTTCGAGGATCACATGCTCCGTACACGTTCGGAGGG |
| | | GGGACCAAGCTGGAAATAAAACGG |
| 5 | Amino acid sequence of | QVKLEQSGPGLVAPSQSLSITCTVSGFSLTSYGVHWVRQSPG |
| | IFN-gamma scFv TII | KGLEWLGVIWAGGSTNYNSALMSRLSISKDNSKSQVFLKMNS |
| | 2G1G7 | LQTDDTAMYYCARRDGNRYYTMDYWGQGASVTVSSGGGGSGG |
| | | GGSGGGGSENVLTQSPAIMSASPGEKVTMTCRASSSVSSSYL |
| | | HWYQQKSGGSPKLWIYRTSNLASGVPARFSGSGSGTSYSLTI |
| | | SSVEAEDAATYYCQQYSGYPLTFGAGTKLELKR |
| 6 | An exemplary coding | CAAGTTAAGCTGGAGCAGTCAGGACCTGGCCTGGTGGCGCCC |
| | sequence for the IFN- | TCACAGAGCCTGTCCATCACTTGCACTGTCTCTGGGTTTTCA |
| | gamma scFv TII 2G1G7 | TTAACCAGCTATGGTGTACACTGGGTTCGCCAGTCTCCAGGA |
| | | AAGGGTCTGGAGTGGCTGGGAGTAATATGGGCTGGTGGAAGC |
| | | ACAAATTATAATTCGGCTCTCATGTCCAGACTGAGCATCAGT |
| | | AAAGACAACTCCAAGAGCCAAGTTTTCTTAAAAATGAACAGT |
| | | CTGCAAACTGATGACACAGCCATGTACTACTGTGCCAGAAGA |
| | | GATGGTAACAGGTACTATACTATGGACTACTGGGGTCAAGGA |
| | | GCTTCAGTCACCGTCTCCTCAGGTGGTGGTGGGTCTGGCGGA |
| | | GGTGGTAGTGGAGGTGGTGGGTCTGAAAATGTGCTCACCCAG |
| | | TCTCCAGCAATCATGTCTGCATCTCCAGGGGAAAAGGTCACC |
| | | ATGACCTGCAGGGCCAGCTCAAGTGTAAGTTCCAGTTACTTG |
| | | CACTGGTACCAGCAGAAGTCAGGTGGCTCCCCCAAACTCTGG |
| | | ATTTATAGAACATCCAACTTGGCTTCTGGAGTCCCTGCTCGC |
| | | TTCAGTGGCAGTGGGTCTGGGACCTCTTACTCTCTCACAATC |
| | | AGCAGTGTGGAGGCTGAAGATGCTGCCACTTATTACTGCCAG |
| | | CAGTACAGTGGTTACCCGCTCACGTTCGGTGCTGGGACCAAG |
| | | CTGGAGCTGAAACGG |
| 7 | Exemplary amino acid | IEVMYPPPYLDNEKSNGTIIHVKGKHLCPSPLFPGPSKP |
| | sequence of a CD28 hinge | |
| 8 | Exemplary amino acid | RVKFSRSADAPAYQQGQNQLYNELNLGRREEYDVLDKRRGRD |
| | sequence of an | PEMGGKPRRKNPQEGLYNELQKDKMAEAYSEIGMKGERRRGK |
| | intracellular signalling | GHDGLYQGLSTATKDTYDALHMQALPPR |
| | domain of CD3 zeta | |
| 9 | Exemplary amino acid | RSKRSRLLHSDYMNMTPRRLGPTRKHYQPYAPPRDFAAYRS |
| | sequence of the co- | |
| | stimulatory intracellular | |
| | signalling domain of CD28 | |
| 10 | Exemplary amino acid | FWVLVVVGGVLACYSLLVTVAFIIFWV |
| | sequence of a CD28 | |
| | transmembrane domain | |
| 11 | Exemplary coding | ATGGCTTTGCCTGTGACTGCACTGTTGCTTCCCTTGGCTCTG |
| | sequence of a CAR | CTTCTCCATGCTGCTAGACCGCAAGTTAAGCTGGAGCAGTCA |
| | comprising IFN-gamma | GGACCTGGCCTGGTGGCGCCCTCACAGAGCCTGTCCATCACT |
| | scFv TII 2G1G7, a IgG4 | TGCACTGTCTCTGGGTTTTCATTAACCAGCTATGGTGTACAC |
| | hinge region, a CD28 | TGGGTTCGCCAGTCTCCAGGAAAGGGTCTGGAGTGGCTGGGA |
| | transmembrane domain, a | GTAATATGGGCTGGTGGAAGCACAAATTATAATTCGGCTCTC |
| | CD28 co-stimulatory | ATGTCCAGACTGAGCATCAGTAAAGACAACTCCAAGAGCCAA |
| | domain, and a CD3 zeta | GTTTTCTTAAAAATGAACAGTCTGCAAACTGATGACACAGCC |
| | signalling domain | ATGTACTACTGTGCCAGAAGAGATGGTAACAGGTACTATACT |
| | | ATGGACTACTGGGGTCAAGGAGCTTCAGTCACCGTCTCCTCA |
| | | GGTGGTGGTGGGTCTGGCGGAGGTGGTAGTGGAGGTGGTGGG |
| | | TCTGAAAATGTGCTCACCCAGTCTCCAGCAATCATGTCTGCA |
| | | TCTCCAGGGGAAAAGGTCACCATGACCTGCAGGGCCAGCTCA |
| | | AGTGTAAGTTCCAGTTACTTGCACTGGTACCAGCAGAAGTCA |
| | | GGTGGCTCCCCCAAACTCTGGATTTATAGAACATCCAACTTG |
| | | GCTTCTGGAGTCCCTGCTCGCTTCAGTGGCAGTGGGTCTGGG |
| | | ACCTCTTACTCTCTCACAATCAGCAGTGTGGAGGCTGAAGAT |
| | | GCTGCCACTTATTACTGCCAGCAGTACAGTGGTTACCCGCTC |
| | | ACGTTCGGTGCTGGGACCAAGCTGGAGCTGAAACGGGCTAGC |
| | | GCAGAAAGCAAGTATGGTCCTCCCTGCCCCCCGTGCCCATTT |
| | | TGGGTGCTGGTGGTGGTTGGGGGAGTCCTGGCTTGCTATAGC |
| | | TTGCTAGTAACAGTGGCCTTTATTATTTTCTGGGTGAGGAGT |
| | | AAGAGGAGCAGGCTCCTGCACAGTGACTACATGAACATGACT |
| | | CCAAGACGCCTAGGTCCCACTCGCAAGCATTACCAGCCCTAT |
| | | GCCCCACCACGCGACTTCGCAGCCTATCGCTCCAGAGTGAAG |
| | | TTCAGCAGGAGCGCAGACGCTCCAGCTTACCAGCAGGGCCAG |
| | | AACCAGCTCTATAACGAGCTCAATCTAGGACGAAGAGAGGAG |
| | | TACGATGTTTTGGACAAGAGACGTGGCCGGGACCCTGAGATG |
| | | GGGGGAAAGCCGAGAAGGAAGAACCCTCAGGAAGGCCTGTAC |
| | | AATGAACTGCAGAAAGATAAGATGGCGGAGGCCTACAGTGAG |
| | | ATTGGGATGAAAGGCGAGCGCCGGAGGGGCAAGGGGCACGAT |
| | | GGCCTTTACCAGGGTCTCAGTACAGCCACCAAGGACACCTAC |
| | | GACGCCCTTCACATGCAGGCCCTGCCCCCTCGCTAA |
| 12 | Exemplary amino acid | MALPVTALLLPLALLLHAARPEVQLLESGGGLVQPGGSLRLS |
| | sequence of CAR PMC882 | CAASGFTFSSYAMSWVRQAPGKGLEWVSSISSTGASTTYADS |
| | comprising the TNF alpha | VKGRFTISRDNSKNTLYLQMNSLRAEDTAVYYCAKGGAAFDY |
| | scFv, a CD28 hinge, a | WGQGTLVTVSSGGGGSGGGGSGGGGSDIQMTQSPSSLSASVG |
| | CD28 transmembrane | DRVTITCRASQSISSYLNWYQQKPGKAPKLLIYSASYLQSGV |
| | domain, a CD28 co- | PSRFSGSGSGTDFTLTISSLQPEDFATYYCQQANNAPTTFGQ |
| | stimulatory domain, and a | GTKVEIKASAIEVMYPPPYLDNEKSNGTIIHVKGKHLCPSPL |
| | CD3 zeta signalling | FPGPSKPFWVLVVVGGVLACYSLLVTVAFIIFWVRSKRSRLL |
| | domain (with CD8a leader | HSDYMNMTPRRLGPTRKHYQPYAPPRDFAAYRSRVKFSRSAD |
| | sequence underlined) | APAYQQGQNQLYNELNLGRREEYDVLDKRRGRDPEMGGKPRR |
| | | KNPQEGLYNELQKDKMAEAYSEIGMKGERRRGKGHDGLYQGL |
| | | STATKDTYDALHMQALPPR |
| 13 | Exemplary coding | GGAGGAAAAACTGTTTCATACAGAAGGCGTGGAGGAAAAACT |
| | sequence of an NFAT- | GTTTCATACAGAAGGCGTGGAGGAAAAACTGTTTCATACAGA |
| | inducible expression | AGGCGTGGAGGAAAAACTGTTTCATACAGAAGGCGTGGAGGA |
| | cassette, encoding a | AAAACTGTTTCATACAGAAGGCGTGGAGGAAAAACTGTTTCA |
| | 6(NFAT) minimal IL-2 | TACAGAAGGCGTCAGAATTAACAGTATAAATTGCATCTCTTG |
| | promoter, a TGF-beta | TTCAAGAGTTCCCTATCACTCGAGCGGTCGCCACCATGCCAC |
| | gene, a T2A peptide | CTTCTGGACTGCGGTTGCTTCCGTTGCTGTTGCCACTGCTTT |
| | separating the TGF-beta | GGCTGCTTGTTCTGACACCTGGCAGACCCGCAGCCGGTTTGT |
| | and IL-10, and an IL-10 | CAACATGCAAAACGATCGACATGGAGCTGGTAAAGCGCAAGC |
| | gene | GCATTGAGGCAATCAGAGGGCAGATCTTGAGCAAGCTTCGGC |
| | | TCGCCTCTCCTCCAAGCCAGGGCGAGGTGCCACCCGGTCCGC |
| | | TGCCGGAAGCGGTTCTCGCTCTCTATAACAGCACTAGAGATA |
| | | GGGTCGCAGGTGAGTCTGCTGAACCTGAACCTGAACCGGAAG |
| | | CTGACTATTACGCTAAAGAGGTGACGAGAGTTCTCATGGTGG |
| | | AAACTCACAATGAGATTTATGATAAATTTAAACAGTCTACCC |
| | | ACAGCATTTATATGTTTTTTAACACCAGTGAGCTCCGCGAGG |
| | | CTGTCCCTGAACCTGTCCTCCTTTCCAGGGCAGAATTGCGGC |
| | | TTCTGAGGCTGAAGCTTAAAGTAGAGCAACACGTGGAGCTCT |
| | | ACCAGAAATATAGTAATAACAGCTGGAGATATCTGTCAAACC |
| | | GACTGCTGGCGCCCTCCGACAGTCCAGAGTGGCTTTCCTTTG |
| | | ACGTTACTGGCGTGGTGCGGCAATGGCTTTCAAGAGGCGGGG |
| | | AAATCGAGGGCTTCAGGCTTAGCGCTCACTGCTCATGTGATA |
| | | GTAGGGATAACACACTTCAGGTAGACATTAATGGGTTCACGA |
| | | CAGGAAGAAGAGGAGATCTGGCTACGATTCATGGCATGAATA |
| | | GGCCTTTCCTCCTGCTTATGGCTACGCCATTGGAGCGCGCGC |
| | | AACATCTCCAGTCCAGCCGCCACAGACGAGCTCTGGATACTA |
| | | ACTACTGCTTCTCCAGCACTGAGAAGAACTGCTGCGTAAGAC |
| | | AGCTTTACATCGACTTCCGCAAGGACCTGGGTTGGAAGTGGA |
| | | TACACGAGCCCAAGGGATATCATGCCAACTTCTGCCTCGGGC |
| | | CCTGTCCCTACATCTGGTCATTGGATACACAGTACAGCAAGG |
| | | TACTCGCTCTGTACAACCAGCACAACCCTGGTGCAAGCGCAG |
| | | CTCCCTGCTGTGTTCCCCAGGCTCTGGAACCATTGCCTATTG |
| | | TTTATTATGTAGGACGAAAACCGAAGGTCGAGCAGCTCAGTA |
| | | ACATGATCGTTCGGAGCTGCAAGTGCTCTGGCTCAGGCGAGG |
| | | GCAGAGGCAGCCTGCTGACATGTGGCGACGTGGAAGAGAACC |
| | | CCGGGCCTAGTCCAGGTCAAGGTACACAGAGTGAGAACAGTT |
| | | GCACACACTTTCCCGGCAACTTGCCTAATATGCTGAGGGACC |
| | | TCCGGGATGCTTTCTCTCGGGTAAAAACTTTTTTTCAAATGA |
| | | AAGACCAACTCGATAACCTCCTTCTGAAGGAGAGCCTCCTGG |
| | | AGGACTTCAAGGGATATTTGGGGTGCCAAGCTTTGTCCGAGA |
| | | TGATTCAGTTCTACTTGGAAGAGGTGATGCCACAAGCGGAGA |
| | | ACCAAGACCCGGACATCAAGGCTCACGTCAATAGCTTGGGAG |
| | | AAAACCTGAAGACCCTGCGGTTGAGGCTGCGCCGCTGTCATA |
| | | GGTTCCTTCCATGTGAGAACAAATCCAAAGCTGTGGAACAGG |
| | | TCAAAAATGCTTTCAACAAACTGCAGGAGAAAGGTATATATA |
| | | AGGCCATGTCTGAATTTGACATATTTATCAACTACATAGAGG |
| | | CGTATATGACCATGAAGATTCGCAAT |
| 14 | Full coding sequence of | ATGGCTTTGCCTGTGACTGCACTGTTGCTTCCCTTGGCTCTG |
| | the CAR construct | CTTCTCCATGCTGCTAGACCGGACTATAAAGATGACGATGAT |
| | comprising the TNF-alpha | AAGGGCGGGTCCgaggtgcagctggtggagtctgggggaggc |
| | scFv, IgG4 hinge, CD28 | ttggtacagcccggcaggtccctgagactctcctgtgcggcc |
| | transmembrane domain, | tctggattcacctttgatgattatgccatgcactgggtccgg |
| | CD28 co-stimulatory | caagctccagggaagggcctggaatgggtctcagctatcact |
| | domain, CD3 zeta | tggaatagtggtcacatagactatgcggactctgtggagggc |
| | signalling domain with a | cgattcaccatctccagagacaacgccaagaactccctgtat |
| | T2A skip peptide to | ctgcaaatgaacagtctgagagctgaggatacggccgtatat |
| | FOXP3 in the same | tactgtgcgaaagtctcgtaccttagcaccgcgtcctccctt |
| | cassette and a NFAT | gactattggggccaaggtaccctggtcaccgtctcgagtGGT |
| | inducible expression | GGTGGTGGGTCTGGCGGAGGTGGTAGTGGAGGTGGTGGGTCT |
| | cassette encoding TGF- | gacatccagatgacccagtctccatcctccctgtctgcatct |
| | beta and IL10 | gtaggggacagagtcaccatcacttgtcgggcaagtcagggc |
| | Upper and lowercase | atcagaaattacttagcctggtatcagcaaaaaccagggaaa |
| | characteristic denote | gcccctaagctcctgatctatgctgcatccactttgcaatca |
| | boundaries of different | ggggtcccatctcggttcagtggcagtggatctgggacagat |
| | components of the CAR | ttcactctcaccatcagcagcctacagcctgaagatgttgca |
| | summarised above | acttattactgtcaaaggtataaccgtgcaccgtatactttt |
| | | ggccagggcaccaaggtggaaatcaaaGCTAGCGCAGAAAGC |
| | | AAGTATGGTCCTCCCTGCCCCCCGTGCCCATTTTGGGTGCTG |
| | | GTGGTGGTTGGGGGAGTCCTGGCTTGCTATAGCTTGCTAGTA |
| | | ACAGTGGCCTTTATTATTTTCTGGGTGAGGAGTAAGAGGAGC |
| | | AGGCTCCTGCACAGTGACTACATGAACATGACTCCAAGACGC |
| | | CTAGGTCCCACTCGCAAGCATTACCAGCCCTATGCCCCACCA |
| | | CGCGACTTCGCAGCCTATCGCTCCAGAGTGAAGTTCAGCAGG |
| | | AGCGCAGACGCTCCAGCTTACCAGCAGGGCCAGAACCAGCTC |
| | | TATAACGAGCTCAATCTAGGACGAAGAGAGGAGTACGATGTT |
| | | TTGGACAAGAGACGTGGCCGGGACCCTGAGATGGGGGGAAAG |
| | | CCGAGAAGGAAGAACCCTCAGGAAGGCCTGTACAATGAACTG |
| | | CAGAAAGATAAGATGGCGGAGGCCTACAGTGAGATTGGGATG |
| | | AAAGGCGAGCGCCGGAGGGGCAAGGGGCACGATGGCCTTTAC |
| | | CAGGGTCTCAGTACAGCCACCAAGGACACCTACGACGCCCTT |
| | | CACATGCAGGCCCTGCCCCCTCGCGGCTCAGGCGAGGGCAGA |
| | | GGCAGCCTGCTGACATGTGGCGACGTGGAAGAGAACCCCGGG |
| | | CCTatgcctaatcctcggcctggaaagcctagcgctccttct |
| | | cttgctctgggaccttctcctggcgcctctccatcttggaga |
| | | gccgctcctaaagccagcgatctgctgggagctagaggacct |
| | | ggcggcacatttcagggcagagatcttagaggcggagcccac |
| | | gctagctcctccagccttaatcctatgcctcctagccagctc |
| | | cagctgcctacactgcctctggttatggtggctcctagcgga |
| | | gctagactgggccctctgcctcatctgcaagctctgctgcag |
| | | gacagaccccacttcatgcaccagctgagcaccgtggatgcc |
| | | cacgcaagaacacctgtgctgcaggttcaccctctggaatcc |
| | | ccagccatgatcagcctgacacctccaacaacagccaccggc |
| | | gtgttcagcctgaaagccagacctggactgcctcctggcatc |
| | | aatgtggccagcctggaatgggtgtccagagaacctgctctg |
| | | ctgtgcacattccccaatccaagcgctcccagaaaggacagc |
| | | acactgtctgccgtgcctcagagcagctatcccctgcttgct |
| | | aacggcgtgtgcaagtggcctggatgcgagaaggtgttcgag |
| | | gaacccgaggacttcctgaagcactgccaggccgatcatctg |
| | | ctggacgagaaaggcagagcccagtgtctgctccagcgcgag |
| | | atggtgcagtctctggaacagcagctggtcctggaaaaagaa |
| | | aagctgagcgccatgcaggcccacctggccggaaaaatggcc |
| | | ctgacaaaggccagcagcgtggcctcttctgataagggcagc |
| | | tgctgcattgtggccgctggatctcagggacctgtggttcct |
| | | gcttggagcggacctagagaggcccctgattctctgtttgcc |
| | | gtgcggagacacctgtggggctctcacggcaactctactttc |
| | | cccgagttcctgcacaacatggactacttcaagttccacaac |
| | | atgeggcctccattcacctacgccacactgatcagatgggcc |
| | | attctggaagcccctgagaagcagagaaccctgaacgagatc |
| | | taccactggtttacccggatgttcgccttcttccggaatcac |
| | | cctgccacctggaagaacgccatccggcacaatctgagcctg |
| | | cacaagtgcttcgtgcgcgtggaatctgagaaaggcgccgtg |
| | | tggacagtggacgagctggaattcagaaagaagagaagccag |
| | | cggcctagccggtgcagcaatcctacacctggacctTAATAG |
| | | ACGCGTGGGTTAGGGGAGGCGCTTTTCCCAAGGCAGTCTGGA |
| | | GCATGCGCTAAgcgatcgcgCCCGCTGGGCACTTGGCGCTAC |
| | | ACAAGTGGCCTCTGGCCTCGCACACATTCCAGGAGGAAAAAC |
| | | TGTTTCATACAGAAGGCGTGGAGGAAAAACTGTTTCATACAG |
| | | AAGGCGTGGAGGAAAAACTGTTTCATACAGAAGGCGTGGAGG |
| | | AAAAACTGTTTCATACAGAAGGCGTGGAGGAAAAACTGTTTC |
| | | ATACAGAAGGCGTGGAGGAAAAACTGTTTCATACAGAAGGCG |
| | | TCAGAATTAACAGTATAAATTGCATCTCTTGTTCAAGAGTTC |
| | | CCTATCACTCGAGCGGTCGCCACCATGCCACCTTCTGGACTG |
| | | CGGTTGCTTCCGTTGCTGTTGCCACTGCTTTGGCTGCTTGTT |
| | | CTGACACCTGGCAGACCCGCAGCCGGTTTGTCAACATGCAAA |
| | | ACGATCGACATGGAGCTGGTAAAGCGCAAGCGCATTGAGGCA |
| | | ATCAGAGGGCAGATCTTGAGCAAGCTTCGGCTCGCCTCTCCT |
| | | CCAAGCCAGGGCGAGGTGCCACCCGGTCCGCTGCCGGAAGCG |
| | | GTTCTCGCTCTCTATAACAGCACTAGAGATAGGGTCGCAGGT |
| | | GAGTCTGCTGAACCTGAACCTGAACCGGAAGCTGACTATTAC |
| | | GCTAAAGAGGTGACGAGAGTTCTCATGGTGGAAACTCACAAT |
| | | GAGATTTATGATAAATTTAAACAGTCTACCCACAGCATTTAT |
| | | ATGTTTTTTAACACCAGTGAGCTCCGCGAGGCTGTCCCTGAA |
| | | CCTGTCCTCCTTTCCAGGGCAGAATTGCGGCTTCTGAGGCTG |
| | | AAGCTTAAAGTAGAGCAACACGTGGAGCTCTACCAGAAATAT |
| | | AGTAATAACAGCTGGAGATATCTGTCAAACCGACTGCTGGCG |
| | | CCCTCCGACAGTCCAGAGTGGCTTTCCTTTGACGTTACTGGC |
| | | GTGGTGCGGCAATGGCTTTCAAGAGGCGGGGAAATCGAGGGC |
| | | TTCAGGCTTAGCGCTCACTGCTCATGTGATAGTAGGGATAAC |
| | | ACACTTCAGGTAGACATTAATGGGTTCACGACAGGAAGAAGA |
| | | GGAGATCTGGCTACGATTCATGGCATGAATAGGCCTTTCCTC |
| | | CTGCTTATGGCTACGCCATTGGAGCGCGCGCAACATCTCCAG |
| | | TCCAGCCGCCACAGACGAGCTCTGGATACTAACTACTGCTTC |
| | | TCCAGCACTGAGAAGAACTGCTGCGTAAGACAGCTTTACATC |
| | | GACTTCCGCAAGGACCTGGGTTGGAAGTGGATACACGAGCCC |
| | | AAGGGATATCATGCCAACTTCTGCCTCGGGCCCTGTCCCTAC |
| | | ATCTGGTCATTGGATACACAGTACAGCAAGGTACTCGCTCTG |
| | | TACAACCAGCACAACCCTGGTGCAAGCGCAGCTCCCTGCTGT |
| | | GTTCCCCAGGCTCTGGAACCATTGCCTATTGTTTATTATGTA |
| | | GGACGAAAACCGAAGGTCGAGCAGCTCAGTAACATGATCGTT |
| | | CGGAGCTGCAAGTGCTCTGGCTCAGGCGAGGGCAGAGGCAGC |
| | | CTGCTGACATGTGGCGACGTGGAAGAGAACCCCGGGCCTATG |
| | | cacagctcagcactgctctgttgcctggtcctcctgactggg |
| | | gtgagggccAGTCCAGGTCAAGGTACACAGAGTGAGAACAGT |
| | | TGCACACACTTTCCCGGCAACTTGCCTAATATGCTGAGGGAC |
| | | CTCCGGGATGCTTTCTCTCGGGTAAAAACTTTTTTTCAAATG |
| | | AAAGACCAACTCGATAACCTCCTTCTGAAGGAGAGCCTCCTG |
| | | GAGGACTTCAAGGGATATTTGGGGTGCCAAGCTTTGTCCGAG |
| | | ATGATTCAGTTCTACTTGGAAGAGGTGATGCCACAAGCGGAG |
| | | AACCAAGACCCGGACATCAAGGCTCACGTCAATAGCTTGGGA |
| | | GAAAACCTGAAGACCCTGCGGTTGAGGCTGCGCCGCTGTCAT |
| | | AGGTTCCTTCCATGTGAGAACAAATCCAAAGCTGTGGAACAG |
| | | GTCAAAAATGCTTTCAACAAACTGCAGGAGAAAGGTATATAT |
| | | AAGGCCATGTCTGAATTTGACATATTTATCAACTACATAGAG |
| | | GCGTATATGACCATGAAGATTCGCAATTGA |
| 15 | Full sequence of a CAR | ATGGCTTTGCCTGTGACTGCACTGTTGCTTCCCTTGGCTCTG |
| | construct comprising of | CTTCTCCATGCTGCTAGACCGCAAGTTAAGCTGGAGCAGTCA |
| | IFN-gamma scFv TII | GGACCTGGCCTGGTGGCGCCCTCACAGAGCCTGTCCATCACT |
| | 2G1G7, an IgG4 hinge, | TGCACTGTCTCTGGGTTTTCATTAACCAGCTATGGTGTACAC |
| | CD28 transmembrane | TGGGTTCGCCAGTCTCCAGGAAAGGGTCTGGAGTGGCTGGGA |
| | domain, CD28 co- | GTAATATGGGCTGGTGGAAGCACAAATTATAATTCGGCTCTC |
| | stimulatory domain, CD3 | ATGTCCAGACTGAGCATCAGTAAAGACAACTCCAAGAGCCAA |
| | zeta signalling domain | GTTTTCTTAAAAATGAACAGTCTGCAAACTGATGACACAGCC |
| | with a T2A skip peptide | ATGTACTACTGTGCCAGAAGAGATGGTAACAGGTACTATACT |
| | to FOXP3 in the same | ATGGACTACTGGGGTCAAGGAGCTTCAGTCACCGTCTCCTCA |
| | cassette and a NFAT | GGTGGTGGTGGGTCTGGCGGAGGTGGTAGTGGAGGTGGTGGG |
| | inducible expression | TCTGAAAATGTGCTCACCCAGTCTCCAGCAATCATGTCTGCA |
| | cassette encoding TGF- | TCTCCAGGGGAAAAGGTCACCATGACCTGCAGGGCCAGCTCA |
| | beta and IL10 | AGTGTAAGTTCCAGTTACTTGCACTGGTACCAGCAGAAGTCA |
| | Upper and lowercase | GGTGGCTCCCCCAAACTCTGGATTTATAGAACATCCAACTTG |
| | characteristic denote | GCTTCTGGAGTCCCTGCTCGCTTCAGTGGCAGTGGGTCTGGG |
| | boundaries of different | ACCTCTTACTCTCTCACAATCAGCAGTGTGGAGGCTGAAGAT |
| | components summarised | GCTGCCACTTATTACTGCCAGCAGTACAGTGGTTACCCGCTC |
| | above | ACGTTCGGTGCTGGGACCAAGCTGGAGCTGAAACGGGCTAGC |
| | | GCAGAAAGCAAGTATGGTCCTCCCTGCCCCCCGTGCCCATTT |
| | | TGGGTGCTGGTGGTGGTTGGGGGAGTCCTGGCTTGCTATAGC |
| | | TTGCTAGTAACAGTGGCCTTTATTATTTTCTGGGTGAGGAGT |
| | | AAGAGGAGCAGGCTCCTGCACAGTGACTACATGAACATGACT |
| | | CCAAGACGCCTAGGTCCCACTCGCAAGCATTACCAGCCCTAT |
| | | GCCCCACCACGCGACTTCGCAGCCTATCGCTCCAGAGTGAAG |
| | | TTCAGCAGGAGCGCAGACGCTCCAGCTTACCAGCAGGGCCAG |
| | | AACCAGCTCTATAACGAGCTCAATCTAGGACGAAGAGAGGAG |
| | | TACGATGTTTTGGACAAGAGACGTGGCCGGGACCCTGAGATG |
| | | GGGGGAAAGCCGAGAAGGAAGAACCCTCAGGAAGGCCTGTAC |
| | | AATGAACTGCAGAAAGATAAGATGGCGGAGGCCTACAGTGAG |
| | | ATTGGGATGAAAGGCGAGCGCCGGAGGGGCAAGGGGCACGAT |
| | | GGCCTTTACCAGGGTCTCAGTACAGCCACCAAGGACACCTAC |
| | | GACGCCCTTCACATGCAGGCCCTGCCCCCTCGCGGCTCAGGC |
| | | GAGGGCAGAGGCAGCCTGCTGACATGTGGCGACGTGGAAGAG |
| | | AACCCCGGGCCTatgcctaatcctcggcctggaaagcctagc |
| | | gctccttctcttgctctgggaccttctcctggcgcctctcca |
| | | tcttggagagccgctcctaaagccagcgatctgctgggagct |
| | | agaggacctggcggcacatttcagggcagagatcttagaggc |
| | | ggagcccacgctagctcctccagccttaatcctatgcctcct |
| | | agccagctccagctgcctacactgcctctggttatggtggct |
| | | cctagcggagctagactgggccctctgcctcatctgcaagct |
| | | ctgctgcaggacagaccccacttcatgcaccagctgagcacc |
| | | gtggatgcccacgcaagaacacctgtgctgcaggttcaccct |
| | | ctggaatccccagccatgatcagcctgacacctccaacaaca |
| | | gccaccggcgtgttcagcctgaaagccagacctggactgcct |
| | | cctggcatcaatgtggccagcctggaatgggtgtccagagaa |
| | | cctgctctgctgtgcacattccccaatccaagcgctcccaga |
| | | aaggacagcacactgtctgccgtgcctcagagcagctatccc |
| | | ctgcttgctaacggcgtgtgcaagtggcctggatgcgagaag |
| | | gtgttcgaggaacccgaggacttcctgaagcactgccaggcc |
| | | gatcatctgctggacgagaaaggcagagcccagtgtctgctc |
| | | cagcgcgagatggtgcagtctctggaacagcagctggtcctg |
| | | gaaaaagaaaagctgagcgccatgcaggcccacctggccgga |
| | | aaaatggccctgacaaaggccagcagcgtggcctcttctgat |
| | | aagggcagctgctgcattgtggccgctggatctcagggacct |
| | | gtggttcctgcttggagcggacctagagaggcccctgattct |
| | | ctgtttgccgtgcggagacacctgtggggctctcacggcaac |
| | | tctactttccccgagttcctgcacaacatggactacttcaag |
| | | ttccacaacatgcggcctccattcacctacgccacactgatc |
| | | agatgggccattctggaagcccctgagaagcagagaaccctg |
| | | aacgagatctaccactggtttacccggatgttcgccttcttc |
| | | cggaatcaccctgccacctggaagaacgccatccggcacaat |
| | | ctgagcctgcacaagtgcttcgtgcgcgtggaatctgagaaa |
| | | ggcgccgtgtggacagtggacgagctggaattcagaaagaag |
| | | agaagccagcggcctagccggtgcagcaatcctacacctgga |
| | | cctTAATAGACGCGTGGGTTAGGGGAGGCGCTTTTCCCAAGG |
| | | CAGTCTGGAGCATGCGCTAATTAGCTAGCCCCGCTGGGCACT |
| | | TGGCGCTACACAAGTGGCCTCTGGCCTCGCACACATTCCAGG |
| | | AGGAAAAACTGTTTCATACAGAAGGCGTGGAGGAAAAACTGT |
| | | TTCATACAGAAGGCGTGGAGGAAAAACTGTTTCATACAGAAG |
| | | GCGTGGAGGAAAAACTGTTTCATACAGAAGGCGTGGAGGAAA |
| | | AACTGTTTCATACAGAAGGCGTGGAGGAAAAACTGTTTCATA |
| | | CAGAAGGCGTCAGAATTAACAGTATAAATTGCATCTCTTGTT |
| | | CAAGAGTTCCCTATCACTCGAGCGGTCGCCACCATGCCACCT |
| | | TCTGGACTGCGGTTGCTTCCGTTGCTGTTGCCACTGCTTTGG |
| | | CTGCTTGTTCTGACACCTGGCAGACCCGCAGCCGGTTTGTCA |
| | | ACATGCAAAACGATCGACATGGAGCTGGTAAAGCGCAAGCGC |
| | | ATTGAGGCAATCAGAGGGCAGATCTTGAGCAAGCTTCGGCTC |
| | | GCCTCTCCTCCAAGCCAGGGCGAGGTGCCACCCGGTCCGCTG |
| | | CCGGAAGCGGTTCTCGCTCTCTATAACAGCACTAGAGATAGG |
| | | GTCGCAGGTGAGTCTGCTGAACCTGAACCTGAACCGGAAGCT |
| | | GACTATTACGCTAAAGAGGTGACGAGAGTTCTCATGGTGGAA |
| | | ACTCACAATGAGATTTATGATAAATTTAAACAGTCTACCCAC |
| | | AGCATTTATATGTTTTTTAACACCAGTGAGCTCCGCGAGGCT |
| | | GTCCCTGAACCTGTCCTCCTTTCCAGGGCAGAATTGCGGCTT |
| | | CTGAGGCTGAAGCTTAAAGTAGAGCAACACGTGGAGCTCTAC |
| | | CAGAAATATAGTAATAACAGCTGGAGATATCTGTCAAACCGA |
| | | CTGCTGGCGCCCTCCGACAGTCCAGAGTGGCTTTCCTTTGAC |
| | | GTTACTGGCGTGGTGCGGCAATGGCTTTCAAGAGGCGGGGAA |
| | | ATCGAGGGCTTCAGGCTTAGCGCTCACTGCTCATGTGATAGT |
| | | AGGGATAACACACTTCAGGTAGACATTAATGGGTTCACGACA |
| | | GGAAGAAGAGGAGATCTGGCTACGATTCATGGCATGAATAGG |
| | | CCTTTCCTCCTGCTTATGGCTACGCCATTGGAGCGCGCGCAA |
| | | CATCTCCAGTCCAGCCGCCACAGACGAGCTCTGGATACTAAC |
| | | TACTGCTTCTCCAGCACTGAGAAGAACTGCTGCGTAAGACAG |
| | | CTTTACATCGACTTCCGCAAGGACCTGGGTTGGAAGTGGATA |
| | | CACGAGCCCAAGGGATATCATGCCAACTTCTGCCTCGGGCCC |
| | | TGTCCCTACATCTGGTCATTGGATACACAGTACAGCAAGGTA |
| | | CTCGCTCTGTACAACCAGCACAACCCTGGTGCAAGCGCAGCT |
| | | CCCTGCTGTGTTCCCCAGGCTCTGGAACCATTGCCTATTGTT |
| | | TATTATGTAGGACGAAAACCGAAGGTCGAGCAGCTCAGTAAC |
| | | ATGATCGTTCGGAGCTGCAAGTGCTCTGGCTCAGGCGAGGGC |
| | | AGAGGCAGCCTGCTGACATGTGGCGACGTGGAAGAGAACCCC |
| | | GGGCCTAGTCCAGGTCAAGGTACACAGAGTGAGAACAGTTGC |
| | | ACACACTTTCCCGGCAACTTGCCTAATATGCTGAGGGACCTC |
| | | CGGGATGCTTTCTCTCGGGTAAAAACTTTTTTTCAAATGAAA |
| | | GACCAACTCGATAACCTCCTTCTGAAGGAGAGCCTCCTGGAG |
| | | GACTTCAAGGGATATTTGGGGTGCCAAGCTTTGTCCGAGATG |
| | | ATTCAGTTCTACTTGGAAGAGGTGATGCCACAAGCGGAGAAC |
| | | CAAGACCCGGACATCAAGGCTCACGTCAATAGCTTGGGAGAA |
| | | AACCTGAAGACCCTGCGGTTGAGGCTGCGCCGCTGTCATAGG |
| | | TTCCTTCCATGTGAGAACAAATCCAAAGCTGTGGAACAGGTC |
| | | AAAAATGCTTTCAACAAACTGCAGGAGAAAGGTATATATAAG |
| | | GCCATGTCTGAATTTGACATATTTATCAACTACATAGAGGCG |
| | | TATATGACCATGAAGATTCGCAATTGA |
| 16 | Exemplary amino acid | SPGQGTQSENSCTHFPGNLPNMLRDLRDAFSRVKTFFQMKDQ |
| | sequence of IL-10 | LDNLLLKESLLEDFKGYLGCQALSEMIQFYLEEVMPQAENQD |
| | | PDIKAHVNSLGENLKTLRLRLRRCHRFLPCENKSKAVEQVKN |
| | | AFNKLQEKGIYKAMSEFDIFINYIEAYMTMKIRN |
| 17 | Exemplary amino acid | PPSGLRLLPLLLPLLWLLVLTPGRPAAGLSTCKTIDMELVKR |
| | sequence of TGF-beta | KRIEAIRGQILSKLRLASPPSQGEVPPGPLPEAVLALYNSTR |
| | | DRVAGESAEPEPEPEADYYAKEVTRVLMVETHNEIYDKFKQS |
| | | THSIYMFFNTSELREAVPEPVLLSRAELRLLRLKLKVEQHVE |
| | | LYQKYSNNSWRYLSNRLLAPSDSPEWLSFDVTGVVRQWLSRG |
| | | GEIEGFRLSAHCSCDSRDNTLQVDINGFTTGRRGDLATIHGM |
| | | NRPFLLLMATPLERAQHLQSSRHRRALDTNYCFSSTEKNCCV |
| | | RQLYIDFRKDLGWKWIHEPKGYHANFCLGPCPYIWSLDTQYS |
| | | KVLALYNQHNPGASAAPCCVPQALEPLPIVYYVGRKPKVEQL |
| | | SNMIVRSCKCS |
| 18 | Exemplary amino acid | MPNPRPGKPSAPSLALGPSPGASPSWRAAPKASDLLGARGPG |
| | sequence of FOXP3 | GTFQGRDLRGGAHASSSSLNPMPPSQLQLPTLPLVMVAPSGA |
| | | RLGPLPHLQALLQDRPHFMHQLSTVDAHARTPVLQVHPLESP |
| | | AMISLTPPTTATGVFSLKARPGLPPGINVASLEWVSREPALL |
| | | CTFPNPSAPRKDSTLSAVPQSSYPLLANGVCKWPGCEKVFEE |
| | | PEDFLKHCQADHLLDEKGRAQCLLQREMVQSLEQQLVLEKEK |
| | | LSAMQAHLAGKMALTKASSVASSDKGSCCIVAAGSQGPVVPA |
| | | WSGPREAPDSLFAVRRHLWGSHGNSTFPEFLHNMDYFKFHNM |
| | | RPPFTYATLIRWAILEAPEKQRTLNEIYHWFTRMFAFFRNHP |
| | | ATWKNAIRHNLSLHKCFVRVESEKGAVWTVDELEFRKKRSQR |
| | | PSRCSNPTPGP* |
| 19 | anti-TNF-alpha scFv clone | EVQLLESGGGLVQPGGSLRLSCAASGFTFSSYAMSWVRQAPG |
| | P3H2 VH | KGLEWVSSISSTGASTTYADSVKGRFTISRDNSKNTLYLQMN |
| | | SLRAEDTAVYYCAKGGAAFDYWGQGTLVTVSS |
| 20 | anti-TNF-alpha scFv clone | DIQMTQSPSSLSASVGDRVTITCRASQSISSYLNWYQQKPGK |
| | P3H2 VL | APKLLIYSASYLQSGVPSRFSGSGSGTDFTLTISSLQPEDFA |
| | | TYYCQQANNAPTTFGQGTKVEIK |
| 21 | anti-TNF-alpha scFv clone | SYAMS |
| | P3H2 & Anti-IFNy | |
| | NovImmune Clone A6 - | |
| | Emapalumab - CDRH1 | |
| | (Kabat) | |
| 22 | anti-TNF-alpha scFv clone | GFTFSSYAMS |
| | P3H2 & Anti-IFNy | |
| | NovImmune Clone A6 - | |
| | Emapalumab - CDRH1 | |
| | (AbM) | |
| 23 | anti-TNF-alpha scFv clone | GFTFSSYA |
| | P3H2 & Anti-IFNy | |
| | NovImmune Clone A6 - | |
| | Emapalumab - CDRH1 | |
| | (IMGT) | |
| 24 | anti-TNF-alpha scFv clone | SISSTGASTTYADSVKG |
| | P3H2 - CDRH2 (Kabat) | |
| 25 | anti-TNF-alpha scFv clone | SISSTGASTT |
| | P3H2 - CDRH2 (AbM) | |
| 26 | anti-TNF-alpha scFv clone | ISSTGAST |
| | P3H2 - CDRH2 (IMGT) | |
| 27 | anti-TNF-alpha scFv clone | GGAAFDY |
| | P3H2 - CDRH3 (Kabat, | |
| | AbM) | |
| 28 | anti-TNF-alpha scFv clone | AKGGAAFDY |
| | P3H2 - CDRH3 (IMGT) | |
| 29 | anti-TNF-alpha scFv clone | RASQSISSYLN |
| | P3H2 - CDRLI (Kabat, | |
| | AbM) | |
| 30 | anti-TNF-alpha scFv clone | QSISSY |
| | P3H2 - CDRLI (IMGT) | |
| 31 | anti-TNF-alpha scFv clone | SASYLOS |
| | P3H2 - CDRL2 (Kabat, | |
| | AbM) | |
| 32 | anti-TNF-alpha scFvclone | SA |
| | P3H2 - CDRL2 (IMGT) | |
| 33 | anti-TNF-alpha scFv clone | QQANNAPTT |
| | P3H2 - CDRL3 (Kabat, | |
| | AbM, IMGT) | |
| 34 | IFN-gamma scFv TII | EVKLEQSGTVLARPGASVKMSCKASGYTFTNYWMHWIRQRPG |
| | 6AE52 VH | QGLEWIGAIYPGISDTSYNQKFKVKAKLTAVTSTSTAYMELS |
| | | SLTNEDSAVYYCTRDEDFDGFAYWGQGTLVTVSA |
| 35 | IFN-gamma scFv TII | DVLMTQTPLSLPVSLGDQASISCRSSQSIVHSNGNTYLDWYL |
| | 6AE52 VL | QKPGQSPKLLIYKVSNRFSGVPDRFSGSGSGTDFTLRISRVE |
| | | AEDLGVYYCFRGSHAPYTFGGGTKLEIKR |
| 36 | IFN-gamma scFv TII | NYWMH |
| | 6AE52 - CDRH1 (Kabat) | |
| 37 | IFN-gamma scFv TII | GYTFTNYWMH |
| | 6AE52 - CDRH1 (AbM) | |
| 38 | IFN-gamma scFv TII | GYTFTNYW |
| | 6AE52 - CDRH1 (IMGT) | |
| 39 | IFN-gamma scFv TII | AIYPGISDTSYNQKFKV |
| | 6AE52 - CDRH2 (Kabat) | |
| 40 | IFN-gamma scFv TII | AIYPGISDTS |
| | 6AE52 - CDRH2 (AbM) | |
| 41 | IFN-gamma scFv TII | IYPGISDT |
| | 6AE52 - CDRH2 (IMGT) | |
| 42 | IFN-gamma scFv TII | DEDFDGFAY |
| | 6AE52 - CDRH3 (Kabat, | |
| | AbM) | |
| 43 | IFN-gamma scFv TII | TRDEDFDGFAY |
| | 6AE52 - CDRH3 (IMGT) | |
| 44 | IFN-gamma scFv TII | RSSQSIVHSNGNTYLD |
| | 6AE52 - CDRL1 (Kabat, | |
| | AbM) | |
| 45 | IFN-gamma scFv TII | QSIVHSNGNTY |
| | 6AE52 - CDRL1 (IMGT) | |
| 46 | IFN-gamma scFv TII | KVSNRFS |
| | 6AE52 - CDRL2 (Kabat, | |
| | AbM) | |
| 47 | IFN-gamma scFv TII | KV |
| | 6AE52 & Anti-IL17A | |
| | Clone Mab126 - | |
| | Ixekizumab - CDRL2 | |
| | (IMGT) | |
| 48 | IFN-gamma scFv TII | FRGSHAPYT |
| | 6AE52 - CDRL3 (Kabat, | |
| | AbM, IMGT) | |
| 49 | IFN-gamma scFv TII | QVKLEQSGPGLVAPSQSLSITCTVSGFSLTSYGVHWVRQSPG |
| | 2G1G7 VH | KGLEWLGVIWAGGSTNYNSALMSRLSISKDNSKSQVFLKMNS |
| | | LQTDDTAMYYCARRDGNRYYTMDYWGQGASVTVSS |
| 50 | IFN-gamma scFv TII | ENVLTQSPAIMSASPGEKVTMTCRASSSVSSSYLHWYQQKSG |
| | 2G1G7 VL | GSPKLWIYRTSNLASGVPARFSGSGSGTSYSLTISSVEAEDA |
| | | ATYYCQQYSGYPLTFGAGTKLELKR |
| 51 | IFN-gamma scFv TII | SYGVH |
| | 2G1G7 - CDRH1 (Kabat) | |
| 52 | IFN-gamma scFv TII | GFSLTSYGVH |
| | 2G1G7 - CDRH1 (AbM) | |
| 53 | IFN-gamma scFv TII | GFSLTSYG |
| | 2G1G7 - CDRH1 (IMGT) | |
| 54 | IFN-gamma scFv TII | VIWAGGSTNYNSALMS |
| | 2G1G7 - CDRH2 (Kabat) | |
| 55 | IFN-gamma scFv TII | VIWAGGSTN |
| | 2G1G7 - CDRH2 (AbM) | |
| 56 | IFN-gamma scFv TII | IWAGGST |
| | 2G1G7 - CDRH2 (IMGT) | |
| 57 | IFN-gamma scFv TII | RDGNRYYTMDY |
| | 2G1G7 - CDRH3 (Kabat, | |
| | AbM) | |
| 58 | IFN-gamma scFv TII | ARRDGNRYYTMDY |
| | 2G1G7 - CDRH3 (IMGT) | |
| 59 | IFN-gamma scFv TII | RASSSVSSSYLH |
| | 2G1G7 - CDRL1 (Kabat, | |
| | AbM) | |
| 60 | IFN-gamma scFv TII | SSVSSSY |
| | 2G1G7 - CDRL1 (IMGT) | |
| 61 | IFN-gamma scFv TII | RTSNLAS |
| | 2G1G7 - CDRL2 (Kabat, | |
| | AbM) | |
| 62 | IFN-gamma scFv TII | RT |
| | 2G1G7 - CDRL2 (IMGT) | |
| 63 | IFN-gamma scFv TII | QQYSGYPLT |
| | 2G1G7 - CDRL3 (Kabat, | |
| | AbM, IMGT) | |
| 64 | Exemplary amino acid | EVQLVESGGGLVQPGRSLRLSCAASGFTFDDYAMHWVRQAPG |
| | sequence of anti-TNFa | KGLEWVSAITWNSGHIDYADSVEGRFTISRDNAKNSLYLQMN |
| | scFv clone 2DE7 | SLRAEDTAVYYCAKVSYLSTASSLDYWGQGTLVTVSSGGGGS |
| | | GGGGSGGGGSDIQMTQSPSSLSASVGDRVTITCRASQGIRNY |
| | | LAWYQQKPGKAPKLLIYAASTLQSGVPSRFSGSGSGTDFTLT |
| | | ISSLQPEDVATYYCQRYNRAPYTFGQGTKVEIK |
| 65 | Anti-TNFa scFv clone | EVQLVESGGGLVQPGRSLRLSCAASGFTFDDYAMHWVRQAPG |
| | Humira 2DE7 VH | KGLEWVSAITWNSGHIDYADSVEGRFTISRDNAKNSLYLQMN |
| | | SLRAEDTAVYYCAKVSYLSTASSLDYWGQGTLVTVSS |
| 66 | Anti-TNFa scFv clone | DIQMTQSPSSLSASVGDRVTITCRASQGIRNYLAWYQQKPGK |
| | Humira 2DE7 VL | APKLLIYAASTLQSGVPSRFSGSGSGTDFTLTISSLQPEDVA |
| | | TYYCQRYNRAPYTFGQGTKVEIK |
| 67 | Anti-TNFa scFv clone | DYAMH |
| | Humira 2DE7 & Anti- | |
| | TNFa scFv clone 2SD4- | |
| | CDRH1 (Kabat) | |
| 68 | Anti-TNFa scFv clone | GFTFDDYAMH |
| | Humira 2DE7 & Anti- | |
| | TNFa scFv clone 2SD4- | |
| | CDRH1 (AbM) | |
| 69 | Anti-TNFa scFv clone | GFTFDDYA |
| | Humira 2DE7 & Anti- | |
| | TNFa scFv clone 2SD4 - | |
| | CDRH1 (IMGT) | |
| 70 | Anti-TNFa scFv clone | AITWNSGHIDYADSVEG |
| | Humira 2DE7 & Anti- | |
| | TNFa scFv clone 2SD4- | |
| | CDRH2 (Kabat) | |
| 71 | Anti-TNFa scFv clone | AITWNSGHID |
| | Humira 2DE7 & Anti- | |
| | TNFa scFv clone 2SD4 - | |
| | CDRH2 (AbM) | |
| 72 | Anti-TNFa scFv clone | ITWNSGHI |
| | Humira 2DE7 & Anti- | |
| | TNFa scFv clone 2SD4 - | |
| | CDRH2 (IMGT) | |
| 73 | Anti-TNFa scFv clone | VSYLSTASSLDY |
| | Humira 2DE7 - CDRH3 | |
| | (Kabat, AbM) | |
| 74 | Anti-TNFa scFv clone | AKVSYLSTASSLDY |
| | Humira 2DE7 - CDRH3 | |
| | (IMGT) | |
| 75 | Anti-TNFa scFv clone | RASQGIRNYLA |
| | Humira 2DE7 & Anti- | |
| | TNFa scFv clone 2SD4 - | |
| | CDRL1 (Kabat, AbM) | |
| 76 | Anti-TNFa scFv clone | QGIRNY |
| | Humira 2DE7 & Anti- | |
| | TNFa scFv clone 2SD4 - | |
| | CDRL1 (IMGT) | |
| 77 | Anti-TNFa scFv clone | AASTLQS |
| | Humira 2DE7 & Anti- | |
| | TNFa scFv clone 2SD4 - | |
| | CDRL2 (Kabat, AbM) | |
| 78 | Anti-TNFa scFv clone | AA |
| | Humira 2DE7 & Anti- | |
| | TNFa scFv clone 2SD4 - | |
| | CDRL2 (IMGT) | |
| 79 | Anti-TNFa scFv clone | QRYNRAPYT |
| | Humira 2DE7 - CDRL3 | |
| | (Kabat, AbM, IMGT) | |
| 80 | Anti-TNFa scFv clone | QVQLVESGGGLVQPGRSLRLSCAASGFTFDDYAMHWVRQAPG |
| | 2SD4 VH | KGLDWVSAITWNSGHIDYADSVEGRFAVSRDNAKNALYLQMN |
| | | SLRPEDTAVYYCTKASYLSTSSLDNWGQGTLVTVSS |
| 81 | Anti-TNFa scFv clone | DIQMTQSPSSLSASIGDRVTITVRASQGIRNYLAWYQQKPGK |
| | 2SD4 VL | APKLLIYAASTLQSGVPSRFSGSGSGTDFTLTISSLQPEDVA |
| | | TYYCQKYNSAPYAFGQGTKVEIK |
| 82 | Anti-TNFa scFv clone | ASYLSTSSLDN |
| | 2SD4 - CDRH3 (Kabat, | |
| | AbM) | |
| 83 | Anti-TNFa scFv clone | TKASYLSTSSLDN |
| | 2SD4 - CDRH3 (IMGT) | |
| 84 | Anti-TNFa scFv clone | QKYNSAPYA |
| | 2SD4 - CDRL3 (Kabat, | |
| | AbM, IMGT) | |
| 85 | Anti-TNFa scFv clone cA2 - | EVKLEESGGGLVQPGGSMKLSCVASGFIFSNHWMNWVRQSPE |
| | Infliximab VH | KGLEWVAEIRSKSINSATHYAESVKGRFTISRDDSKSAVYLQ |
| | | MTDLRTEDTGVYYCSRNYYGSTYDYWGQGTTLTVS |
| 86 | Anti-TNFa scFv clone cA2 - | DILLTQSPAILSVSPGERVSFSCRASQFVGSSIHWYQQRTNG |
| | Infliximab VL | SPRLLIKYASESMSGIPSRFSGSGSGTDFTLSINTVESEDIA |
| | | DYYCQQSHSWPFTFGSGTNLEVK |
| 87 | Anti-TNFa scFv clone cA2 - | NHWMN |
| | Infliximab - CDRH1 | |
| | (Kabat) | |
| 88 | Anti-TNFa scFv clone cA2 - | GFIFSNHWMN |
| | Infliximab - CDRH1 | |
| | (AbM) | |
| 89 | Anti-TNFa scFv clone cA2 - | GFIFSNHW |
| | Infliximab - CDRH1 | |
| | (IMGT) | |
| 90 | Anti-TNFa scFv clone cA2 - | EIRSKSINSATHYAESVKG |
| | Infliximab - CDRH2 | |
| | (Kabat) | |
| 91 | Anti-TNFa scFv clone cA2 - | EIRSKSINSATH |
| | Infliximab - CDRH2 | |
| | (AbM) | |
| 92 | Anti-TNFa scFv clone cA2 - | IRSKSINSAT |
| | Infliximab - CDRH2 | |
| | (IMGT) | |
| 93 | Anti-TNFa scFv clone cA2 - | NYYGSTYDY |
| | Infliximab - CDRH3 | |
| | (Kabat, AbM) | |
| 94 | Anti-TNFa scFv clone cA2 - | SRNYYGSTYDY |
| | Infliximab - CDRH3 | |
| | (IMGT) | |
| 95 | Anti-TNFa scFv clone cA2 - | RASQFVGSSIH |
| | Infliximab - CDRL1 | |
| | (Kabat, AbM) | |
| 96 | Anti-TNFa scFv clone cA2 - | QFVGSS |
| | Infliximab - CDRL1 | |
| | (IMGT) | |
| 97 | Anti-TNFa scFv clone cA2 - | YASESMS |
| | Infliximab - CDRL2 | |
| | (Kabat, AbM) | |
| 98 | Anti-TNFa scFv clone cA2 - | YA |
| | Infliximab - CDRL2 | |
| | (IMGT) | |
| 99 | Anti-TNFa scFv clone cA2 - | QQSHSWPFT |
| | Infliximab - CDRL3 | |
| | (Kabat, AbM, IMGT) | |
| 100 | Anti-IFNy NovImmune | EVQLLESGGGLVQPGGSLRLSCAASGFTFSSYAMSWVRQAPG |
| | Clone A6 - Emapalumab | KGLEWVSAISGSGGSTYYADSVKGRFTISRDNSKNTLYLQMN |
| | VH | SLRAEDTAVYYCAKDGSSGWYVPHWFDPWGRGTLVTVSS |
| 101 | Anti-IFNy NovImmune | NFMLTQPHSVSESPGKTVTISCTRSSGSIVSNYVQWYQQRPG |
| | Clone A6 - Emapalumab | SAPTTVIYEDNRRPSGVPDRFSGSIDSSSNTASLTISGLEAE |
| | VL | DEADYYCQSYDGSNRWMFGGGTKLTVLG |
| 102 | Anti-IFNy NovImmune | AISGSGGSTYYADSVKG |
| | Clone A6 - Emapalumab - | |
| | CDRH2 (Kabat) | |
| 103 | Anti-IFNy NovImmune | AISGSGGSTY |
| | Clone A6 - Emapalumab - | |
| | CDRH2 (AbM) | |
| 104 | Anti-IFNy NovImmune | ISGSGGST |
| | Clone A6 - Emapalumab - | |
| | CDRH2 (IMGT) | |
| 105 | Anti-IFNy NovImmune | DGSSGWYVPHWFDP |
| | Clone A6 - Emapalumab - | |
| | CDRH3 (Kabat, AbM) | |
| 106 | Anti-IFNy NovImmune | AKDGSSGWYVPHWFDP |
| | Clone A6 - Emapalumab - | |
| | CDRH3 (IMGT) | |
| 107 | Anti-IFNy NovImmune | TRSSGSIVSNYVQ |
| | Clone A6 - Emapalumab - | |
| | CDRL1 (Kabat, AbM) | |
| 108 | Anti-IFNy NovImmune | SGSIVSNY |
| | Clone A6 - Emapalumab - | |
| | CDRL1 (IMGT) | |
| 109 | Anti-IFNy NovImmune | EDNRRPS |
| | Clone A6 - Emapalumab - | |
| | CDRL2 (Kabat, AbM) | |
| 110 | Anti-IFNy NovImmune | ED |
| | Clone A6 - Emapalumab - | |
| | CDRL2 (IMGT) | |
| 111 | Anti-IFNy NovImmune | QSYDGSNRWM |
| | Clone A6 - Emapalumab - | |
| | CDRL3 (Kabat, AbM, | |
| | IMGT) | |
| 112 | Anti-IFNy HuZAF - | QVQLVQSGAELKKPGSSVKVSCKASGYIFTSSWINWVQAPGQ |
| | Fontolizumab VH | GLEWIGRIDPSDGEVHYNQDFKDKATLTTVDKSTNTAYMELS |
| | | SLRSEDTAVYYCARGFLPWFADWGQGTLVTVSS |
| 113 | Anti-IFNy HuZAF - | DIQMTQSPSTLSASVGDRVTITCKASENVDTYVSWYQQKPGK |
| | Fontolizumab VL | APKLLIYGASNRYTGVPSRFSGSGSGTDFTLTISSLQPDDFA |
| | | TYYCGQSYNYPFTFGQGTKVEVKR |
| 114 | Anti-IFNy HuZAF - | SSWIN |
| | Fontolizumab - CDRH1 | |
| | (Kabat) | |
| 115 | Anti-IFNy HuZAF - | GYIFTSSWIN |
| | Fontolizumab - CDRH1 | |
| | (AbM) | |
| 116 | Anti-IFNy HuZAF - | GYIFTSSW |
| | Fontolizumab - CDRH1 | |
| | (IMGT) | |
| 117 | Anti-IFNy HuZAF - | RIDPSDGEVHYNQDFKD |
| | Fontolizumab - CDRH2 | |
| | (Kabat) | |
| 118 | Anti-IFNy HuZAF - | IDPSDGEV |
| | Fontolizumab - CDRH2 | |
| | (AbM) | |
| 119 | Anti-IFNy HuZAF - | RIDPSDGEVH |
| | Fontolizumab - CDRH2 | |
| | (IMGT) | |
| 120 | Anti-IFNy HuZAF - | GFLPWFAD |
| | Fontolizumab - CDRH3 | |
| | (Kabat, AbM) | |
| 121 | Anti-IFNy HuZAF - | ARGFLPWFAD |
| | Fontolizumab - CDRH3 | |
| | (IMGT) | |
| 122 | Anti-IFNy HuZAF - | KASENVDTYVS |
| | Fontolizumab - CDRL1 | |
| | (Kabat, AbM) | |
| 123 | Anti-IFNy HuZAF - | ENVDTY |
| | Fontolizumab - CDRL1 | |
| | (IMGT) | |
| 124 | Anti-IFNy HuZAF - | GASNRYT |
| | Fontolizumab - CDRL2 | |
| | (Kabat, AbM) | |
| 125 | Anti-IFNy HuZAF - | GA |
| | Fontolizumab & Anti- | |
| | IL17A Clone AIN457 - | |
| | Secukinumab - CDRL2 | |
| | (IMGT) | |
| 126 | Anti-IFNy HuZAF - | GQSYNYPFT |
| | Fontolizumab - CDRL3 | |
| | (Kabat, AbM, IMGT) | |
| 127 | Anti-IL17A Clone AIN457 - | EVQLVESGGGLVQPGGSLRLSCAASGFTFSNYWMNWVRQAPG |
| | Secukinumab VH | KGLEWVAAINQDGSEKYYVGSVKGRFTISRDNAKNSLYLQMN |
| | | SLRVEDTAVYYCVRDYYDILTDYYIHYWYFDLWGRGTLVTVS |
| | | S |
| 128 | Anti-IL17A Clone AIN457 - | EIVLTQSPGTLSLSPGERATLSCRASQSVSSSYLAWYQQKPG |
| | Secukinumab VL | QAPRLLIYGASSRATGIPDRFSGSGSGTDFTLTISRLEPEDF |
| | | AVYYCQQYGSSPCTFGQGTRLEIKR |
| 129 | Anti-IL17A Clone AIN457 - | NYWMN |
| | Secukinumab - CDRH1 | |
| | (Kabat) | |
| 130 | Anti-IL17A Clone AIN457 - | GFTFSNYWMN |
| | Secukinumab - CDRH1 | |
| | (AbM) | |
| 131 | Anti-IL17A Clone AIN457 - | GFTFSNYW |
| | Secukinumab - CDRH1 | |
| | (IMGT) | |
| 132 | Anti-IL17A Clone AIN457 - | AINQDGSEKYYVGSVKG |
| | Secukinumab - CDRH2 | |
| | (Kabat) | |
| 133 | Anti-IL17A Clone AIN457 - | AINQDGSEKY |
| | Secukinumab - CDRH2 | |
| | (AbM) | |
| 134 | Anti-IL17A Clone AIN457 - | INQDGSEK |
| | Secukinumab - CDRH2 | |
| | (IMGT) | |
| 135 | Anti-IL17A Clone AIN457 - | DYYDILTDYYIHYWYFDL |
| | Secukinumab - CDRH3 | |
| | (Kabat, AbM) | |
| 136 | Anti-IL17A Clone AIN457 - | VRDYYDILTDYYIHYWYFDL |
| | Secukinumab - CDRH3 | |
| | (IMGT) | |
| 137 | Anti-IL17A Clone AIN457 - | RASQSVSSSYLA |
| | Secukinumab - CDRL1 | |
| | (Kabat, AbM) | |
| 138 | Anti-IL17A Clone AIN457 - | QSVSSSY |
| | Secukinumab - CDRL1 | |
| | (IMGT) | |
| 139 | Anti-IL17A Clone AIN457 - | GASSRAT |
| | Secukinumab - CDRL2 | |
| | (Kabat, AbM) | |
| 140 | Anti-IL17A Clone AIN457 - | QQYGSSPCT |
| | Secukinumab - CDRL3 | |
| | (Kabat, AbM, IMGT) | |
| 141 | Anti-IL17A Clone | QVQLVQSGAEVKKPGSSVKVSCKASGYSFTDYHIHWVRQAPG |
| | Mab 126 - Ixekizumab VH | QGLEWMGVINPMYGTTDYNQRFKGRVTITADESTSTAYMELS |
| | | SLRSEDTAVYYCARYDYFTGTGVYWGQGTLVTVSS |
| 142 | Anti-IL17A Clone | DIVMTQTPLSLSVTPGQPASISCRSSRSLVHSRGNTYLHWYL |
| | Mab 126 - Ixekizumab VL | QKPGQSPQLLIYKVSNRFIGVPDRFSGSGSGTDFTLKISRVE |
| | | AEDVGVYYCSQSTHLPFTFGQGTKLEIK |
| 143 | Anti-IL17A Clone | DYHIH |
| | Mab 126 - Ixekizumab - | |
| | CDRH1 (Kabat) | |
| 144 | Anti-IL17A Clone | GYSFTDYHIH |
| | Mab 126 - Ixekizumab - | |
| | CDRH1 (AbM) | |
| 145 | Anti-IL17A Clone | GYSFTDYH |
| | Mab 126 - Ixekizumab - | |
| | CDRH1 (IMGT) | |
| 146 | Anti-IL17A Clone | VINPMYGTTDYNQRFKG |
| | Mab 126 - Ixekizumab - | |
| | CDRH2 (Kabat) | |
| 147 | Anti-IL17A Clone | VINPMYGTTD |
| | Mab 126 - Ixekizumab - | |
| | CDRH2 (AbM) | |
| 148 | Anti-IL17A Clone | INPMYGTT |
| | Mab 126 - Ixekizumab - | |
| | CDRH2 (IMGT) | |
| 149 | Anti-IL17A Clone | YDYFTGTGVY |
| | Mab 126 - Ixekizumab - | |
| | CDRH3 (Kabat, AbM) | |
| 150 | Anti-IL17A Clone | ARYDYFTGTGVY |
| | Mab 126 - Ixekizumab - | |
| | CDRH3 (IMGT) | |
| 151 | Anti-IL17A Clone | RSSRSLVHSRGNTYLH |
| | Mab 126 - Ixekizumab - | |
| | CDRL1 (Kabat, AbM) | |
| 152 | Anti-IL17A Clone | RSLVHSRGNTY |
| | Mab 126 - Ixekizumab - | |
| | CDRL1 (IMGT) | |
| 153 | Anti-IL17A Clone | KVSNRFI |
| | Mab 126 - Ixekizumab - | |
| | CDRL2 (Kabat, AbM) | |
| 154 | Anti-IL17A Clone | SQSTHLPFT |
| | Mab 126 - Ixekizumab - | |
| | CDRL3 (Kabat, AbM, | |
| | IMGT) | |
| 155 | Exemplary amino acid | IYIWAPLAGTCGVLLLSLVITLYC |
| | sequence of a CD8 | |
| | transmembrane domain | |
| 156 | Exemplary amino acid | ESKYGPPCPPCP |
| | sequence of a IgG4 hinge | |
| 157 | Exemplary amino acid | RWPESPKAQASSVPTAQPQAEGSLAKATTAPATTRNTGRGGE |
| | sequence of a IgD hinge | EKKKEKEKEEQEERETKTPECPSHTQPLGVYLLTPAVQDLWL |
| | | RDKATFTCFVVGSDLKDAHLTWEVAGKVPTGGVEEGLLERHS |
| | | NGSQSQHSRLTLPRSLWNAGTSVTCTLNHPSLPPQRLMALRE |
| | | PAAQAPVKLSLNLLASSDPPEAASWLLCEVSGFSPPNILLMW |
| | | LEDQREVNTSGFAPARPPPQPGSTTFWAWSVLRVPAPPSPQP |
| | | ATYTCVVSHEDSRTLLNASRSLEVSYVTDH |
| 158 | Exemplary amino acid | KRGRKKLLYIFKQPFMRPVQTTQEEDGCSCRFPEEEEGGCEL |
| | sequence of a 4-1BB co- | |
| | stimulatrory intracellular | |
| | signalling domain | |
| 159 | Exemplary amino acid | QRRKYRSNKGESPVEPAEPCRYSCPREEEGSTIPIQEDYRKP |
| | sequence of a CD27 co- | EPACSP |
| | stimulatory intracellular | |
| | signalling domain | |
| 160 | Exemplary amino acid | MALPVTALLLPLALLLHAARPQVKLEQSGPGLVAPSQSLSIT |
| | sequence of a CAR | CTVSGFSLTSYGVHWVRQSPGKGLEWLGVIWAGGSTNYNSAL |
| | comprising IFN-gamma | MSRLSISKDNSKSQVFLKMNSLQTDDTAMYYCARRDGNRYYT |
| | scFv TII 2G1G7, a IgG4 | MDYWGQGASVTVSSGGGGSGGGGSGGGGSENVLTQSPAIMSA |
| | hinge region, a CD28 | SPGEKVTMTCRASSSVSSSYLHWYQQKSGGSPKLWIYRTSNL |
| | transmembrane domain, a | ASGVPARFSGSGSGTSYSLTISSVEAEDAATYYCQQYSGYPL |
| | CD28 co-stimulatory | TFGAGTKLELKRASAESKYGPPCPPCPFWVLVVVGGVLACYS |
| | domain, and a CD3 zeta | LLVTVAFIIFWVRSKRSRLLHSDYMNMTPRRLGPTRKHYQPY |
| | signalling domain (with | APPRDFAAYRSRVKFSRSADAPAYQQGQNQLYNELNLGRREE |
| | leader sequence) | YDVLDKRRGRDPEMGGKPRRKNPQEGLYNELQKDKMAEAYSE |
| | | IGMKGERRRGKGHDGLYQGLSTATKDTYDALHMQALPPR |
| 161 | Exemplary amino acid | EVQLLESGGGLVQPGGSLRLSCAASGFTFSSYAMSWVRQAPG |
| | sequence of CAR PMC882 | KGLEWVSSISSTGASTTYADSVKGRFTISRDNSKNTLYLQMN |
| | comprising the TNF alpha | SLRAEDTAVYYCAKGGAAFDYWGQGTLVTVSSGGGGSGGGGS |
| | scFv, a CD28 hinge, a | GGGGSDIQMTQSPSSLSASVGDRVTITCRASQSISSYLNWYQ |
| | CD28 transmembrane | QKPGKAPKLLIYSASYLQSGVPSRFSGSGSGTDFTLTISSLQ |
| | domain, a CD28 co- | PEDFATYYCQQANNAPTTFGQGTKVEIKASAIEVMYPPPYLD |
| | stimulatory domain, and a | NEKSNGTIIHVKGKHLCPSPLFPGPSKPFWVLVVVGGVLACY |
| | CD3 zeta signalling | SLLVTVAFIIFWVRSKRSRLLHSDYMNMTPRRLGPTRKHYQP |
| | domain (without leader | YAPPRDFAAYRSRVKFSRSADAPAYQQGQNQLYNELNLGRRE |
| | sequence) | EYDVLDKRRGRDPEMGGKPRRKNPQEGLYNELQKDKMAEAYS |
| | | EIGMKGERRRGKGHDGLYQGLSTATKDTYDALHMQALPPR |
| 162 | Anti-TNFa scFv clone | AEVQLLESGGGLVQPGGSLRLSCAASGFTFSSYAMSWVRQAP |
| | P3H2 VH | GKGLEWVSSISSTGASTTYADSVKGRFTISRDNSKNTLYLQM |
| | | NSLRAEDTAVYYCAKGGAAFDYWGQGTLVTVSS |
| 163 | Anti-TNFa scFv clone | TDIQMTQSPSSLSASVGDRVTITCRASQSISSYLNWYQQKPG |
| | P3H2 VL | KAPKLLIYSASYLQSGVPSRFSGSGSGTDFTLTISSLQPEDF |
| | | ATYYCQQANNAPTTFGQGTKVEIKRAAA |
| 164 | Exemplary amino acid | TTTPAPRPPTPAPTIASQPLSLRPEACRPAAGGAVHTRGLDF |
| | sequence of a CD8a hinge | ACD |
| 165 | Exemplary amino acid | QVKLEQSGPGLVAPSQSLSITCTVSGFSLTSYGVHWVRQSPG |
| | sequence of a CAR | KGLEWLGVIWAGGSTNYNSALMSRLSISKDNSKSQVFLKMNS |
| | comprising IFN-gamma | LQTDDTAMYYCARRDGNRYYTMDYWGQGASVTVSSGGGGSGG |
| | scFv TII 2G1G7, a IgG4 | GGSGGGGSENVLTQSPAIMSASPGEKVTMTCRASSSVSSSYL |
| | hinge region, a CD28 | HWYQQKSGGSPKLWIYRTSNLASGVPARFSGSGSGTSYSLTI |
| | transmembrane domain, a | SSVEAEDAATYYCQQYSGYPLTFGAGTKLELKRASAESKYGP |
| | CD28 co-stimulatory | PCPPCPFWVLVVVGGVLACYSLLVTVAFIIFWVRSKRSRLLH |
| | domain, and a CD3 zeta | SDYMNMTPRRLGPTRKHYQPYAPPRDFAAYRSRVKFSRSADA |
| | signalling domain (without | PAYQQGQNQLYNELNLGRREEYDVLDKRRGRDPEMGGKPRRK |
| | leader sequence) | NPQEGLYNELQKDKMAEAYSEIGMKGERRRGKGHDGLYQGLS |
| | | TATKDTYDALHMQALPPR |
| 166 | Exemplary amino acid | AEVQLLESGGGLVQPGGSLRLSCAASGFTFSSYAMSWVRQAP |
| | sequence of a TNF-alpha | GKGLEWVSSISSTGASTTYADSVKGRFTISRDNSKNTLYLQM |
| | scFv clone P3H2 - v2 | NSLRAEDTAVYYCAKGGAAFDYWGQGTLVTVSSGGGGSGGGG |
| | | SGGGGSTDIQMTQSPSSLSASVGDRVTITCRASQSISSYLNW |
| | | YQQKPGKAPKLLIYSASYLQSGVPSRFSGSGSGTDFTLTISS |
| | | LQPEDFATYYCQQANNAPTTFGQGTKVEIKRAAA |

### Definitions

The term “polynucleotide” or “nucleic acid” as used herein is defined as a chain of nucleotides. The terms “nucleic acids” and “polynucleotides” (or singular versions), as used herein, are interchangeable. A polynucleotide or nucleic acid may be DNA, RNA or a combination of DNA and RNA. A person skilled in the art would readily understand that nucleic acids are polynucleotides which can be hydrolysed into the monomeric “nucleotides.”

The term “antibody,” as used herein, refers to an immunoglobulin molecule which specifically binds with an antigen. Antibodies can be intact immunoglobulins (e.g., IgG, IgM, IgA, IgD and IgE). derived from natural sources or from recombinant sources and can be immunoreactive portions of intact immunoglobulins. The term “antibody” is used herein in the broadest sense and encompasses various antibody structures, including, but not limited to, monoclonal antibodies, polyclonal antibodies, multispecific antibodies (e.g., bispecific, trispecific antibodies), multivalent antibodies (e.g., bivalent, trivalent antibodies), single chain antibodies, antibody fragments (i.e., Fv, Fab, F(ab)2, etc.), and humanized antibodies., so long as they exhibit the desired antigen-binding activity.

As used herein, the term “vector” refers to any molecule used to transfer coding information to a host cell. Numerous vectors are known in the art including, but not limited to, nuclear acids, linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDRH1, CDRH2 and CDRH3, respectively, wherein CDRH1 corresponds to CDR1 of VH, CDRH2 corresponds to CDR2 of VH and CDRH3 corresponds to CDR3 of VH. Likewise, the CDRs of VL are referred to herein as CDRL1, CDRL2 and CDRL3, respectively, wherein CDRL1 corresponds to CDR1 of VL, CDRL2 corresponds to CDR2 of VL and CDRL3 corresponds to CDR3 of VL. In one example, the amino acid positions assigned to CDRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs are defined according to the Enhanced Chothia Numbering Scheme, also referred to herein as “the AbM numbering system” (http://www.bioinfo.org.uk/mdex.html; Abhinandan & Martin, Mol. Immunology. 45(14): 3832-3839, 2008). In another example, the amino acid positions assigned to CDRs are defined according to the international ImMunoGeneTics information system (IMGT) (Lefranc, Immunology Today. 18: 509, 1997; Lefranc, The Immunologist. 7: 132-136, 1999). The present invention is not limited to CDRs as defined by the Kabat numbering system, but includes all numbering systems, including but not limited to the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; or the numbering system of Honnegher and Plükthun J. Mol. Biol. 309: 657-670, 2001.

As used herein, the term “inflammation associated factors” refers to any factors known to be involved or associated with inflammation. The inflammation associated factors include, but are not limited to proteins, nucleic acids and lipids. The inflammation associated factors can be inducers or promoters of the inflammation process or can be upregulated and/or secreted as a result of the increased inflammation. In particular examples, the inflammation associated factors are pro-inflammatory cytokines or inflammation-associated chemokines. As used herein, the term “cytokines” refers to small proteins or peptides involved in cell signalling, and are generally secreted by immune cells, such as for example, T cells and macrophages. Pro-inflammatory cytokines are cytokines which are involved in the up-regulation of inflammatory reactions, and include, for example, Interleukin 1 (IL-1), Tumour Necrosis Factor alpha (TNF-alpha), Interferon gamma (IFN− gamma), Interleukin 12 (IL-12), Interleukin 18 (IL-18), Granulocyte-macrophage colony-stimulating factor (GMCSF), Interleukin 6 (IL-6), Interleukin 8 (IL-8), Interleukin 17A (IL-17), and the like. As used herein, the term “chemokines” refers to a specialized type of cytokines primarily known for their function in mediating chemotaxis (i.e., serving as attractants to other cells, particularly immune cells). Inflammation-associated chemokines, also called inflammatory chemokines, play an active role in the inflammatory response, for example, in the attraction of immune cells to the site of inflammation. Examples of inflammation-associated chemokines include, but are not limited to, CXCL1, CXCL2, CXCL9, CXCL10, CXCL11, CXCL16, CCL2-20, and the like.

As used herein, the term “codon redundant sequence” or “codon redundancy”, also known as “codon degeneracy”, refers to the multiplicity of three-base pair codon combinations that specify an amino acid. That is to say, one amino acid can be encoded by different triplets (codons) of nucleic acids. However, one triplet (codon) of nucleic acids can only result in a single amino acid.

The term “encoding” refers to the inherent property of specific sequences of nucleotides in a polynucleotide (or the entire polynucleotide), such as a gene, a cDNA (including that of a transgene), or an mRNA, to serve as templates for synthesis of other polymers and macromolecules in biological processes. The polymers and macromolecules have either a defined sequence of nucleotides (i.e., rRNA, tRNA and mRNA) or a defined sequence of amino acids. Thus, a gene encodes a protein if the transcription and translation of the mRNA corresponding to that gene produces said protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA. A polynucleotide is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced therefrom.

As used herein, the term “CRISPR-Cas9” refers to genome editing technology based on the capability of clustered regularly interspaced palindromic repeats (CRISPR) and the CRISPR— associated protein-9 nuclease (Cas9) from, for example, Streptococcus pyogenes to induce, for example, double-strand (ds) DNA breaks in a specific location that is complementary to the synthetic guide RNA (sgRNA) sequence integrated into the CRISPR-Cas9 complex. This allows the deletion, addition, and/or modification of genes and/or other genomic elements, such as transcription elements, promoters, promoter enhancers, transcription enhancers, restriction sites, mutations, selection markers, for example antibiotic selection cassettes, and the like. For example, an antibiotic selection cassette, or any other selection marker, can also added to the genome, preceding, simultaneously with, or following insertion of genetic material using the CRISPR technology. The functions of CRISPR (Clustered Regularly Interspaced Short Palindromic Repeats) and CRISPR-associated (Cas) genes are essential in adaptive immunity in select bacteria and archaea, enabling the organisms to respond to and eliminate invading genetic material. Various types of CRISPR mechanisms have been identified so far, of which type II is the most well-studied. Also, other combinations of CRISPR, for example CRISPR-Cpfl have been developed. Also contemplated herein is the use of CRISPR technologies, wherein the Cas proteins or functionally analogue proteins are not isolated from S. pyogenes. Examples of Cas 9 proteins are, but are not limited to, Cas 9 proteins, or proteins with the same functionality, isolated from S. pyogenees, Staphylococcus aureus, or any representatives of the archaea kingdom (Woese, Kandler & Wheelis, 1990). Cas 9 proteins can also be substituted with so-called CasX and CasY proteins. In another example, examples of Cpfl proteins, or proteins with the same functionality are isolated from, but are not limited to, Acidaminococcus sp. and Lachnospiraceae. In terms of adaptive immunity, the mechanism of CRISPR-Cas9 mediated defence is as follows: invading DNA from viruses or plasmids is cut into small fragments and incorporated into a CRISPR locus amidst a series of short repeats (around 20 bps). The loci are transcribed, and transcripts are then processed to generate small RNAs (crRNA-CRISPR RNA; also referred to as synthetic guide RNA (sgRNA) in an in vitro setting), which are used to guide effector endonucleases that target invading DNA based on sequence complementarity. In terms of gene editing, the CRISPR-Cas9 works according to the same principle, with the sgRNA guiding the effector nucleases to the desired sections of the DNA, in which the excision is to be made.

The term “activation”, as used herein, refers to the state of an immune cell, e.g., a T cell, that has been sufficiently stimulated to show detectable cellular proliferation or cytokine production. In one example as disclosed herein, the activation is the result of stimulation by the binding of the chimeric antigen receptor (CAR) with its one or more target antigens. In a specific example of the present disclosure, wherein the modified T cell comprises a chimeric antigen receptor which is capable of binding with one or more pro-inflammatory cytokines, the binding of the one or more pro-inflammatory cytokines with the chimeric antigen receptor activates the modified T cell via the activity of the signalling domain(s) of the chimeric antigen receptor.

As used herein, the term “immune tolerance” (also known as immunological tolerance, or immunotolerance) refers to a state of unresponsiveness or reduced responsiveness of the immune system to substances that otherwise could elicit an immune response in a human subject, either locally or systematically.

As used herein, the term “autologous” refers to any material derived from the same individual, to which it is later re-introduced. In contrast, the term “allogeneic” refers to a graft derived from a different individual.

As used herein, the term “Tregs” refers to regulatory T cells, a subpopulation of T cells also known as suppressor T cells, which have at least one of the following characteristics: expressing CD4; expressing FOXP3; expressing CD25; CD4+, FOXP3+ and CD25+ T cells; the ability to down regulate the induction and proliferation of effector T cells; CD4+ FOXP3+ CD25 (high) T cells; or a larger T cell receptor (TCR) diversity than effector T cells.

As used herein, the term “autoimmune disease” as used herein is defined as a disease or disorder that is the result of, or results in, a response by the host against itself. An autoimmune disease is therefore the result of an inappropriate and excessive response to a self-antigen. Examples of autoimmune diseases include, but are not limited to, Addision's disease, alopecia greata, ankylosing spondylitis, autoimmune hepatitis, autoimmune parotitis, Crohn's disease, diabetes (Type I), dystrophic epidermolysis bullosa, epididymitis, glomerulonephritis, general rejection of transplanted organs and the like, Graft vs. host disease, Graves' disease, Guillain-Barrè syndrome, Hashimoto's disease, haemolytic anaemia, inflammation, systemic lupus erythematosus (lupus), multiple sclerosis, inflammatory bowel disease, myasthenia gravis, pemphigus vulgaris, psoriatic arthritis, psoriasis, rheumatism, rheumatic fever, rheumatoid arthritis, juvenile rheumatoid arthritis, sarcoidosis, scleroderma, Sjogren's syndrome, spondyloarthropathies, thyroiditis, vasculitis, vitiligo, myxoedema, pernicious anaemia, and ulcerative colitis, among others.

As used herein, the term “operably linked” refers to the linking between a first and a second nucleic acid sequence, whereby the linking allows functional expression of both nucleic acid sequences to take place. In other words, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “subject” as used herein refers to any animal capable of suffering from an autoimmune disease; a transplant rejection, a graft versus host disease (GVHD), a cytokine release syndrome or any disease/condition involving or resulting from uncontrolled inflammatory responses mediated by one or more inflammation associated factor(s). Particular subjects of interest are human beings, and scientifically relevant species such as mice, rats, ferrets, guinea pigs, hamsters, non-human primates, dogs, pigs and sheep, or economically relevant animals such as horses, dogs, cats and cattle. In a preferred embodiment, the subject is a human.

A reference to “providing a subject with” relates to administering to the subject the genetically modified cell. Alternatively, the genetically modified cell may be generated within the subject. For example, the genetically modified cell may be generated in vivo such that the subject has an endogenous population of genetically modified cells. Suitable means for such in vivo generation are known in the art and include gene therapy of a subject.

### DETAILED DESCRIPTION OF THE PRESENT INVENTION

Regulatory T cells (Tregs, a subset of T cells) play an important role in induction and maintenance of peripheral immune tolerance and are key in preventing excessive immune responses and autoimmunity.

Advances in adoptive cell therapy have enabled researchers/clinicians with the ability to endow patients' own immune cells with responsiveness to selected antigen(s) through the introduction of genes that encode synthetic chimeric antigen receptors (CAR).

For this reason, the function of Tregs is utilized according to the present disclosure for dampening or suppressing an immune response. This provides treatment options for subjects with inflammatory disorders, and those undergoing transplantation. As shown herein, potentiation of the inhibitory properties of Tregs, or similar immunosuppressive cells, by gene modification provides modalities for treating autoimmune disorders, transplant rejection, graft versus host disease (GVHD), cytokine release syndrome and any disease/condition involving or resulting from uncontrolled inflammatory responses mediated by one or more inflammation associated factor(s).

Disclosed herein is a strategy of adoptive cell transfer of T cells transduced to express a chimeric antigen receptor (CAR). Instead of binding with tumour-specific antigens and promoting the immune response/activity, as seen in most anti-cancer CART cell therapies, the chimeric antigen receptors disclosed herein bind to, or are capable of binding with, pro-inflammatory cytokines. Upon activation, the modified T cells comprising the chimeric antigen receptor disclosed herein have been shown to lead to the death and/or suppression of effector T cells, thereby suppressing immune responses and activities. Also disclosed herein are materials and methods for regulating inflammatory immune responses and inducing long term drug free immune tolerance in diseases where tolerance is desirable, such as autoimmunity and transplant rejection.

Compositions and methods of the invention uses modified cells, preferably modified regulatory T cells (Tregs) or induced pluripotent stem cells (iPSCs)-derived T cells to modulate immune responses associated with allogenic translations and autoimmune diseases. Modified cells, such as modified Tregs, or iPSC-derived T cells are combined with chimeric antigen receptors (CAR) with a single-chain variable fragment (scFv) that binds to inflammation associated factors which leads to the reduction and resolution of inflammation.

### Chimeric Antigen Receptor

Accordingly, in one aspect, the present disclosure refers to a chimeric antigen receptor (CAR) comprising an extracellular domain, a transmembrane domain, and an intracellular domain, wherein the extracellular domain binds one or more inflammation associated factor(s). In specific examples, the inflammation associated factor(s) are pro-inflammatory cytokines or inflammation—associated chemokines. In a specific example, the one or more inflammation associated factors is/are selected from the group consisting of Interleukin 1 (IL-1), Tumour Necrosis Factor alpha (TNF-alpha), Interferon gamma (IFN-gamma), Interleukin 12 (IL-12), Interleukin 18 (IL-18), Granulocyte-macrophage colony-stimulating factor (GMCSF), Interleukin 6 (IL-6), Interleukin 8 (IL-8), Interleukin 17A (IL-17), CXCL1, CXCL2, CXCL9, CXCL10, CXCL11, CXCL16, and CCL2-20.

In a specific example, the present disclosure refers to a chimeric antigen receptor (CAR) comprising an extracellular domain, a transmembrane domain, and an intracellular domain, wherein the extracellular domain binds one or more pro-inflammatory cytokines. In a further example, the one or more pro-inflammatory cytokines is/are selected from the group consisting of Interleukin 1 (IL-1), Tumour Necrosis Factor alpha (TNF-alpha), Interferon gamma (IFN-gamma), Interleukin 12 (IL-12), Interleukin 18 (IL-18), Granulocyte-macrophage colony-stimulating factor (GMCSF), Interleukin 6 (IL-6), Interleukin 8 (IL-8), and Interleukin 17A (IL-17). In one example, the pro-inflammatory cytokine is Tumour Necrosis Factor alpha (TNF-alpha). In one example, the pro-inflammatory cytokine is Interferon gamma (IFN-gamma). In one example, the pro-inflammatory cytokine is Interleukin 8 (IL-8).

In a specific example, the chimeric antigen receptor as disclosed herein comprises an extracellular domain, a transmembrane domain, and an intracellular domain, wherein the extracellular domain comprises an scFv that binds TNF-alpha, and wherein the hinge region is a CD8a or CD28 hinge, the transmembrane domain is a CD28 transmembrane domain, the signalling domain is the intracellular signalling domain of CD3 zeta, and the co-stimulatory domain is the intracellular signalling domain of CD28. In a specific example, the chimeric antigen receptor as disclosed herein comprises an extracellular domain, a transmembrane domain, and an intracellular domain, wherein the extracellular domain comprises an scFv that binds IFN-gamma, and wherein the hinge region is a CD8a or CD28 hinge, the transmembrane domain is a CD28 transmembrane domain, the signalling domain is the intracellular signalling domain of CD3 zeta, and the co-stimulatory domain is the intracellular signalling domain of CD28. In another specific example, the chimeric antigen receptor as disclosed herein comprises an extracellular domain, a transmembrane domain, and an intracellular domain, wherein the extracellular domain comprises an scFv that binds IL-17a, and wherein the hinge region is a CD8a or CD28 hinge, the transmembrane domain is a CD28 transmembrane domain, the signalling domain is the intracellular signalling domain of CD3 zeta, and the co-stimulatory domain is the intracellular signalling domain of CD28.

### Antigen Binding Domain

In an example, the extracellular domain of the chimeric antigen receptor as disclosed herein comprises one or more antigen binding domains.

The antigen-recognition domain can be any suitable domain that can recognise one or more inflammation associated factor(s) (for example a pro-inflammatory cytokine or inflammation—associated chemokine). As used throughout the specification the term “antigen-recognition domain” refers to the portion of the CAR that provides the specificity of the CAR for the one or more inflammation associated factor(s). The antigen-recognition domain may be all of, or may merely be part of, the extracellular region of the CAR. Suitable antigen-recognition domains, include, but are not limited to, polypeptides having sequence homology to the antigen-binding site of an antibody, or fragment thereof, that bind to an inflammation associated factor. Therefore, in some embodiments of any aspect of the present invention, the antigen-recognition domain includes an amino acid sequence having homology to an antibody, or a fragment thereof, that binds to an inflammation associated factor. In some embodiments, a portion of the antigen-recognition domain includes an amino acid sequence having homology to an antibody, or a fragment thereof, that binds to the inflammation associated factor. The source homologous antibody sequence can be any suitable sequence of an antibody that has an affinity for an inflammation associated factor. For example the sequence can share sequence homology with an antibody originating from one or more of the following species; human, non-human primate, mouse, rat, rabbit, sheep, goat, ferret, canine, chicken, feline, guinea pig, hamster, horse, cow, or pig. The antigen-recognition domain may share sequence homology with the sequence of a monoclonal antibody produced from a hybridoma cell line. When the originating species of the homologous antibody sequence is not human, the antibody is preferably a humanised antibody. The homologous antibody sequence may also be from a non-mammalian animal species such as a cartilaginous fish (e.g. shark IgNAR antibodies—see WO2012/073048). Alternatively, the antigen binding domain may include a modified protein scaffolds that provide functionality similar to shark antibodies, such as i-bodies which have binding moieties based on shark IgNAR antibodies (see WO2005/118629). Additionally, the antigen-recognition domain could be, could be derived from, or could share sequence homology with any other suitable binding molecule or peptide that can selectively interact with an inflammation associated factor with an affinity sufficient to activate the CAR signalling domain. Methods are known in the art for the identification of antigen-binding proteins such as, inter alia, panning phage display libraries, protein affinity chromatography, co-immunoprecipitation and yeast two-hybrid systems (see Srinivasa Rao, V. et al. Int J Proteomics, 2014; article ID 147648).

In some embodiments the antigen-recognition domain of the CAR includes amino acid sequence homology to the amino acid sequence of a fragment-antigen binding (Fab) portion of an antibody that binds to an inflammation associated factor. As will be understood in the art, a Fab portion of an antibody in composed of one constant region and one variable region of each of the heavy and light chains of an antibody. The Fab is the antigen determinant region of the antibody and can be generated by enzymatically cleaving the Fc region from an antibody.

In some embodiments of any aspect of the present invention, the antigen-recognition domain includes amino acid sequence homology to the amino acid sequence of a single-chain variable fragment (scFv) that binds to an inflammation associated factor. As would be understood in the art, a scFv is a fusion protein comprising two portions that may share homology with, or may be identical to, the variable-heavy (VH) and variable-light (VL) chains of an antibody, with the two portions connected together with a linker peptide. For example, the scFv may include VH and VL amino acid sequences that are derived from an antibody that recognises an inflammation associated factor. In this context it will be appreciated that the term “derived from” is not a reference to the source of the polypeptides per se, but rather refers to the derivation of the amino acid sequence that constitute a portion ofthe antigen-binding region. Consequently, the term “derived from” includes synthetically, artificially or otherwise created polypeptides that share sequence identity to an antibody that binds to the inflammation associated factor.

In some embodiments, the antigen-recognition domain includes amino acid sequence homology to the amino acid sequence of a multivalent scFv that binds to an inflammation associated factor. In some embodiments, the multivalent scFv is a di-valent or tri-valent scFv.

In some embodiments of any aspect of the present invention, the antigen-recognition domain has the amino acid sequence of a single-antibody domain (sdAb) that binds to an inflammation associated factor.

In some examples, the/each antigen binding domain is independently selected from the group consisting of an antibody, an antibody fragment, a single-chain variable fragment (scFv), a chemokine receptor, or functional variations thereof. In one specific example, the chemokine receptor is further selected from the group consisting of CCR5, CXCR3, CCR1, and CCR2. In another specific example, the/each antigen binding domain is a single-chain variable fragment (scFv).

In a specific example, the extracellular domain comprises any one or more of the below: a scFv which binds TNF-alpha, a scFv which binds IFN-gamma; and a scFv which binds IL-17a. In a more specific example, the scFv of i) has the amino acid sequence as set forth in SEQ ID: 1; the scFv of ii) has the amino acid sequence as set forth in SEQ ID: 2; and the scFv of iii) has the amino acid sequence as set forth in SEQ ID: 3.

When comparing amino acid sequences, the sequences should be compared over a comparison window which is determined by the length of the polypeptide. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms such as the BLAST family of programs as, for example, disclosed by Altschul et al., 1997, Nucl. Acids Res. 25: 3389-3402. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., 2000, Trends Genet., 16: 276-277), and the GGSEARCH program (available at fasta.bioch.virginia.edu/fasta_www2/fasta_www.cgi?rm=compare&pgm=gnw) which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998).

As indicated above, the functional variant may comprise individual amino acid substitutions, deletions or insertions relative to one of the amino acid sequences described herein, for example in Table 2. For example, a person skilled in the art will recognise that any amino acid can be substituted with a chemically (functionally) similar amino acid and retain function of the polypeptide. Such conservative amino acid substitutions are well known in the art. The following groups in Table 2 each contain amino acids that are conservative substitutions for one another.

TABLE 2
Exemplary amino acid conservative substitutions

| Original | |
|---|---|
| Residue | Exemplary Substitutions |
| Ala (A) | Val (V), Leu (L), Ile (I), Gly (G) |
| Arg (R) | Lys (K) |
| Asn (N) | Gln (Q), His (H) |
| Asp (D) | Glu (E) |
| Cys (C) | Ser (S) |
| Gln (Q) | Asn (N), His (H) |
| Glu (E) | Asp (D) |
| Gly (G) | Pro (P), Ala (A) |
| His (H) | Asn (N), Gln (Q) |
| Ile (I) | Leu (L), Val (V), Ala (A) |
| Leu (L) | Ile (I), Val (V), Met (M), Ala (A), Phe (F) |
| Lys (K) | Arg (R) |
| Met (M) | Leu (L), Phe (F) |
| Phe (F) | Leu (L), Val (V), Alal (A) |
| Pro (P) | Gly (G) |
| Ser (S) | Thr (T) |
| Thr (T) | Ser (S) |
| Trp (W) | Tyr (Y) |
| Tyr (Y) | Trp (W), Phe (F) |
| Val (V) | Ile (I), Leu (L), Met (M), Phe (F), Ala (A) |

Furthermore, if desired, unnatural amino acids or chemical amino acid analogues can be introduced as a substitution or addition into a polypeptide encompassed herein. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, f3-alanine, fluoro-amino acids, designer amino acids such as f3-methyl amino acids, Ca-methyl amino acids, Na-methyl amino acids, and amino acid analogues in general.

### Hinge

In one example, the chimeric antigen receptor as disclosed herein further comprises a hinge region located between the extracellular domain and the transmembrane domain. In a specific example, the hinge region is selected from the group consisting of a CD8a hinge, a CD28 hinge, an IgG hinge, an IgD hinge and functional variations thereof. For example, the hinge region is a CD8a hinge comprising or consisting of an amino acid sequence set forth in SEQ ID NO: 164. In another example, the hinge region is an IgG hinge region, such as an IgG4 hinge region comprising or consisting of an amino acid sequence set forth in SEQ ID NO: 156. In yet another example, the hinge region is an IgD hinge region comprising or consisting of an amino acid sequence set forth in SEQ ID NO: 157. In a preferred example, the hinge region is a CD28 hinge region. In this particular example, the hinge region comprises or consists of an amino acid sequence set forth in SEQ ID NO: 7 or a codon redundant sequence thereof. In certain embodiments, the hinge region comprises or consists of an amino acid sequence having at least 75%, 80%, 85%, 90%, 91%, 92%, 93%, 94%, 95%, 96%, 97%, 98%, 99%, or any percentage in between of identity to the amino acid sequence as set forth in SEQ ID NO: 7, 156, 157 or 164.

### Signalling Domain

In another example, the intracellular domain of the chimeric antigen receptor as disclosed herein comprises a signalling domain and optionally one or more co-stimulatory domains.

In an example, the signaling domain includes a portion derived from an activation receptor.

As used throughout the specification the term “portion”, when used with respect to an activation receptor or co-stimulatory receptor, relates to any segment of the receptor that includes a sequence responsible for, or involved in, the initiation/induction of an intracellular signalling cascade following interaction of the receptor with its cognate antigen or ligand. An example of the initiation/induction of an intracellular signalling cascade for the T cell receptor (TCR) via CD3 is outlined below.

Whilst not wishing to be bound by theory, the extracellular portion ofthe TCR largely comprises heterodimers of either the clonotypic TCRα and TCRβ chains (the TCRα/β receptor) or the TCRγ and TCRδ chains (the TCRγδ receptor). These TCR heterodimers generally lack inherent signalling transduction capabilities and therefore they are non-covalently associated with multiple signal transducing subunits of CD3 (primarily CD3-zeta, -gamma, -delta, and -epsilon). Each of the gamma, delta, and epsilon chains of CD3 has an intracellular (cytoplasmic) portion that includes a single Immune-receptor-Tyrosine-based-Activation-Motif (ITAM), whilst the CD3-zeta chain includes three tandem ITAMs. Upon engagement of the TCR by its cognate antigen in the presence of MHC, and the association of a requisite co-receptor such as CD4 or CD8, signalling is initiated which results in a tyrosine kinase (namely Lek) phosphorylating the two tyrosine residues within the intracellular ITAM(s) of the CD3 chains. Subsequently, a second tyrosine kinase (ZAP-70 -itself activated by Lek phosphorylation) is recruited to biphosphorylate the ITAMs. As a result, several downstream target proteins are activated which eventually leads to intracellular conformational changes, calcium mobilization, and actin cytoskeleton re-arrangement that when combined ultimately lead to activation of transcription factors and induction of a T cell immune response.

As used throughout the specification the term “activation receptor” relates to receptors, or co-receptors that form a component of, or are involved in the formation of, the T cell receptor (TCR) complex, or receptors involved in the specific activation of immune cells as a result of recognition of an antigenic or other immunogenic stimuli.

Non-limiting examples of such activation receptors include components of the T cell receptor-CD3 complex (CD3-zeta, -gamma, -delta, and -epsilon), the CD4 co-receptor, the CD8 co-receptor, FC receptors or Natural Killer (NK) cell associated activation receptors such a LY-49 (KLRA1), natural cytotoxicity receptors (NCR, preferably NKp46, NKp44, NKp30 or NKG2 or the CD94/NKG2 heterodimer). Consequently, in some embodiments, the signalling domain includes a portion derived from any one or more of a member of the CD3 co-receptor complex (preferably the CD3-ζ (chain or a portion thereof), the CD4 co-receptor, the CD8 co-receptor, a Fc Receptor (FcR) (preferably the FcεRI or FcγRI) or NK associated receptors such a LY-49.

In some embodiments of, the signalling domain includes a portion derived from any one of CD3 (preferably the CD3-ζ (chain or a portion thereof) or an FC receptor (preferably the FcεRI or FcγRI).

Various combinations of portions of activation receptors can be utilized to form the transmembrane (TM) and intracellular (IC) portions of the CAR for example the CD3ζ(TM and CD3ζIC (Landmeier S. et al. Cancer Res. 2007; 67:8335-43; Guest R D. et al., J Immunother. 2005, 28:203-11; Hombach A A. et al. J Immunol. 2007; 178: 4650-7), the CD4 TM and CD3 IC (James S E. et al. J Immunol. 2008; 180:7028-38), the CD8 TM and CD3ζIC (Patel S D. et al. Gene Ther. 1999; 6: 412-9), and the FcεRIγ and the FcεRIγ IC (Haynes N M. et al. J Immunol. 2001; 166: 182-7; Annenkov A E. et al. J Immunol. 1998; 161: 6604-13).

In an example, the signalling domain comprises one or more immunoreceptor tyrosine-based activation motifs (ITAMs). In a specific example, the signalling domain comprises an intracellular signalling domain of any one of the proteins selected from the group consisting of TCR zeta, FcR gamma, FcR beta, CD3 gamma, CD3 delta, CD3 epsilon, CD3 zeta, CD22, CD79a, CD79b, CD66d, and functional variations/fragments thereof. In a further specific example, the signalling domain is the intracellular signalling domain of CD3 zeta or a functional variant thereof.

In one specific example, the signalling domain comprises or consists of the amino acid sequence set forth in SEQ ID: 8. In certain embodiments, the signalling domain comprises or consists of an amino acid sequence having at least 75%, 80%, 85%, 90%, 91%, 92%, 93% 94% 95%, 96%, 97%, 98%, 99%, or any percentage in between of identity to the amino acid sequence as set forth in SEQ ID NO: 8 or a codon redundant sequence thereof.

### Co-Stimulatory Domain

As used throughout the specification the term “co-stimulatory receptor” relates to receptors or co-receptors that assist in the activation of an immune cell upon antigen specific inducement of an activation receptor. As will be understood, co-stimulatory receptors do not require the presence of antigen and are not antigen specific, but are typically one of two signals, the other being an activation signal, which is required for the induction of an immune cellular response. In the context of an immune response a co-stimulation receptor is typically activated by the presence of its expressed ligand on the surface of an antigen-presenting cell (APC) such as a dendritic cell or macrophage. With specific regard to T cells, co-stimulation is necessary to lead to cellular activation, proliferation, differentiation and survival (all of which are generally referred to under the umbrella of T cell activation), whilst presentation of an antigen to a T cell in the absence of co-stimulation can lead to anergy, clonal deletion and/or the development of antigen specific tolerance.

Non-limiting examples of T cell co-stimulatory receptors include CD27, CD28, CD30, CD40, DAP10, OX40, 4-1BB (CD137), ICOS. Specifically, CD27, CD28, CD30, CD40, DAP10, OX40, 4-1BB (CD137), and ICOS all represent ‘positive’ co-stimulatory molecules that enhance activation of a T cell response. Accordingly, in some embodiments of any aspect of the present invention, the signalling domain includes a portion derived from any one or more of CD27, CD28, CD30, CD40, DAP10, OX40, 4-1BB (CD137) and ICOS.

In some embodiments of any aspect of the present invention, the signalling domain includes a portion derived from the CD28, OX40 or 4-1BB co-stimulatory receptors. In some embodiments, the signalling domain includes a portion of the CD28 co-stimulatory receptor. In some embodiments, the signalling domain includes a portion of the OX40 co-stimulatory receptor.

Various combinations of portions of co-stimulatory receptors can be utilized to form the transmembrane (TM) and intracellular (IC) portions of the CAR. For example the CD8 TM and DAP10 IC or CD8 TM and 4-1BB IC (Main V. et al. Exp Hematol. 2007; 35: 1388-97), the CD28 TM and the CD28 IC (Wilkie S. et al. J Immunol. 2008;180: 4901-9; Maher J. et al. Nat Biotechnol. 2002; 20: 70-5), and the CD8 TM and the CD28 IC (Marin V. et al. Exp Hematol. 2007; 35: 1388-97).

In one example, each of the one or more co-stimulatory domains comprise an intracellular signalling domain of any one of the proteins selected from the group consisting of: CD28, CD28T, OX40, 4-1BB/CD137, CD2, CD4, CD5, CD7, CD9, CD16, CD22, CD27, CD30, CD33, CD37, CD40, CD45, CD64, CD80, CD86, CD134, CD137, CD154, PD-1, ICOS, lymphocyte function-associated antigen-1 (LFA-1/CD11a/CD18), CD247, CD276 (B7-H3), LIGHT (tumour necrosis factor superfamily member 14; TNFSF14), NKG2C, Ig alpha (CD79a), DAP-10, Fc gamma receptor, MHC class I molecule, TNF, TNFr, integrin, signalling lymphocytic activation molecule, BTLA, Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, IT GAL, CD11a, LFA-1, ITGAM, CD11b, ITGAX, CDllc, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, FOXP3, functional variations/fragments, and combinations thereof. In another example, the intracellular domain comprises FOXP3. In a specific example, each of the one or more co-stimulatory domains comprise an intracellular signalling domain of any one of the proteins selected from the group consisting of CD28, 41BB, IL2Rb, TLR2, MyD88, and CD40. In a preferred example, the one or more co-stimulatory domains comprise the intracellular signalling domain of CD28.

In one specific example, the signalling domain comprises or consists of the amino acid sequence set forth in SEQ ID NO: 9. In certain embodiments, the signalling domain comprises or consists of an amino acid sequence having at least 75%, 80%, 85%, 90%, 91%, 92%, 93% 94% 95%, 96%, 97%, 98%, 99%, or any percentage in between of identity to the amino acid sequence as set forth in SEQ ID: 9 or a codon redundant sequence thereof.

Sequence information for the above-referenced activation and co-stimulatory receptors is readily accessible in a variety of databases. For example, embodiments of human amino acid, gene and mRNA sequences for these receptors is provided in Table 3.

TABLE 3
Summary of Activation and Co-stimulation
Receptor Sequence Information

| | | NCBI | |
|---|---|---|---|
| Receptor | Uniprot | Gene | GeneBank |
| Name | Ref No. | ID No. | mRNA Ref No. |
| CD3-zeta | P20963 | 919 | GI: 166362721 |
| CD3-gamma | P09693 | 917 | GI: 166362738 |
| CD3-delta | P04234 | 915 | GI: 98985799 |
| CD3- | P07766 | 916 | GI: 166362733 |
| epsilon | | | |
| CD4 | P0173 | 920 | GI: 303522473 |
| CD8 alpha | P01732 | 925 | GI: 225007534 |
| CD8 beta | P01966 | 926 | GI: 296010927 |
| FcγRI | P12314 | 2209 | GI: 31331 |
| FcεR1 | Q01362 | 2206 | GI: 219881 |
| Ly-49 | Q7Z556 | 10748 | GI: 33114184 |
| (KLRAI) | | | |
| NKp46 | O76036 | 9437 | GI: 3647268 |
| NKp44 | O95944 | 9436 | GI: 4493701 |
| NKp30 | O14931 | 259197 | GI: 5823969 |
| CD94 | Q13241 | 3824 | GI: 1098616 |
| CD27 | P26842 | 939 | GI: 180084 |
| CD28 | P10747 | 940 | GI: 338444 |
| CD30 | P28908 | 943 | GI: 180095 |
| CD40 | P25942 | 958 | GI: 29850 |
| DAP10 | Q9UBK5 | 10870 | GI: 5738198 |
| OX40 | P43489 | 7293 | GI: 472957 |
| 4-1BB | Q07011 | 3604 | GI: 571320 |
| (CD137) | | | |
| ICOS | Q9Y6W8 | 29851 | GI: 9968295 |
| CTLA-4 | P16410 | 1493 | GI: 291928 |
| PD-1 | Q15116 | 5133 | GI: 2149002 |

Whilst Table 3 is provided with reference to human activation and co-stimulatory receptors, it would be understood by a person skilled in the art that homologous and orthologous versions of each receptor are present in the majority of mammalian and vertebrate species. Therefore, the above-referenced sequences are only provided as non-limiting examples of receptor sequences that may be included in a CAR as described herein and homologous and orthologous sequences from any desired species may be used to generate a CAR that is suitable for the given species.

In one example, the intracellular domain and/or the extracellular domain comprise a self-cleaving peptide. In another example, the self-cleaving peptide is, but is not limited to, P32A, E2A, F2A, and T2A. In yet another example, the self-cleaving peptide is T2A.

### Transmembrane Domain

In another example, the transmembrane domain of the chimeric antigen receptor as disclosed herein is a transmembrane domain of a protein selected from the group consisting of CD28, CD28T, OX-40, 4-1BB/CD137, CD2, CD7, CD27, CD30, CD40, programmed death-l (PD-1), inducible T cell co-stimulator (ICOS), lymphocyte function-associated antigen-1 (LFA-1, CD11a/CD18), CD3 gamma, CD3 delta, CD3 epsilon, CD247, CD276 (B37-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, and functional variations/fragments thereof. In a specific example the transmembrane domain is a CD3 epsilon transmembrane domain or a functional variant thereof.

In a particular example, the transmembrane domain is a CD28 transmembrane domain, preferably the CD28 transmembrane domain comprises or consists of an amino acid sequence set forth in SEQ ID: 10. In certain embodiments, the CD28 transmembrane domain comprises or consists of an amino acid sequence having at least 75%, 80%, 85%, 90%, 91%, 92%, 93%, 94%, 95%, 96%, 97%, 98%, 99%, or any percentage in between of identity to the amino acid sequence as set forth in SEQ ID NO: 10 or a codon redundant sequence thereof.

In a specific example of the chimeric antigen receptor as disclosed herein, the hinge region is an IgG4 hinge, the transmembrane domain is a CD28 transmembrane domain, the signalling domain is the intracellular signalling domain of CD3 zeta, and the co-stimulatory domain is the intracellular signalling domain of CD28.

In a more specific example, the chimeric antigen receptor comprises or consists of the amino acid sequence of SEQ ID NO: 12, SEQ ID NO: 160, SEQ ID NO: 161 or SEQ ID NO: 165, or any other CAR amino acid sequence described in Table 1. In another specific example, the chimeric antigen receptor comprises or consists of the amino acid sequence of SEQ ID NO: 12, SEQ ID NO: 160, SEQ ID NO: 161 or SEQ ID NO: 165 with the “ASA” residues deleted or substituted with any combination of alanine and senne; or any other combination of 3 amino acids. In a more specific example, the chimeric antigen receptor comprises or consists of an amino acid sequence encoded by the nucleotide sequence of SEQ ID NO: 11.

### Nucleic Acids

The present disclosure provides a first polynucleotide, nucleic acid or nucleic acid construct comprising a nucleotide sequence encoding a chimeric antigen receptor as disclosed herein. For example, the nucleic acid may comprise or consist of the nucleotide sequence of SEQ ID NO: 11.

The present disclosure also provides a polynucleotide, nucleic acid or nucleic acid construct comprising or consisting of the nucleotide sequence of SEQ ID NO: 13, SEQ ID NO: 14 or SEQ ID NO: 15.

The present disclosure also provides a polynucleotide, nucleic acid or nucleic acid construct comprising or consisting of the nucleotide sequence that encodes any one or more of the amino acid sequences described in Table 1.

The present disclosure also provides a polynucleotide, nucleic acid or nucleic acid construct that comprises or consists of the nucleotide sequence of a CD28 hinge as shown in SEQ ID NO: 11, or IgG4 hinge as shown in SEQ ID NO: 14 or 15; a CD28 transmembrane domain as shown in SEQ ID NO: 11, 14, or 15; a CD28 co-stimulatory domain as shown in SEQ ID NO: 11, 14, or 15; and/or a CD3 zeta signalling domain as shown in SEQ ID NO: 11, 14, or 15.

The nucleic acid construct may further comprise one or more of: an origin of replication for one or more hosts; a selectable marker gene which is active in one or more hosts; and/or one or more transcriptional control sequences.

As used herein, the term “selectable marker gene” includes any gene that confers a phenotype on a cell in which it is expressed, to facilitate the identification and/or selection of cells which are transfected or transformed with the construct.

“Selectable marker genes” include any nucleotide sequences which, when expressed by a cell transformed with the construct, confer a phenotype on the cell that facilitates the identification and/or selection of these transformed cells. A range of nucleotide sequences encoding suitable selectable markers are known in the art (for example Mortesen, R M. and Kingston R E. Curr Protoc Mol Biol, 2009; Unit 9.5). Exemplary nucleotide sequences that encode selectable markers include: Adenosine deaminase (ADA) gene; Cytosine deaminase (CDA) gene; Dihydrofolate reductase (DHFR) gene; Histidinol dehydrogenase (hisD) gene; Puromycin-N-acetyl transferase (PAC) gene; Thymidine kinase (TK) gene; Xanthine-guanine phosphoribosyltransferase (XGPRT) gene or antibiotic resistance genes such as ampicillin-resistance genes, puromycin-resistance genes, Bleomycin-resistance genes, hygromycin-resistance genes, kanamycin-resistance genes and ampicillin-resistance gene; fluorescent reporter genes such as the green, red, yellow or blue fluorescent protein-encoding genes; and luminescence-based reporter genes such as the luciferase gene, amongst others which permit optical selection of cells using techniques such as Fluorescence-Activated Cell Sorting (FACS).

Furthermore, it should be noted that the selectable marker gene may be a distinct open reading frame in the construct or may be expressed as a fusion protein with another polypeptide (e.g. the CAR).

As set out above, the nucleic acid construct may also comprise one or more transcriptional control sequences. The term “transcriptional control sequence” should be understood to include any nucleic acid sequence which effects the transcription of an operably connected nucleic acid. A transcriptional control sequence may include, for example, a leader, polyadenylation sequence, promoter, enhancer or upstream activating sequence, and transcription terminator. Typically, a transcriptional control sequence at least includes a promoter. The term “promoter” as used herein, describes any nucleic acid which confers, activates or enhances expression of a nucleic acid in a cell.

In some embodiments of any aspect of the present invention, at least one transcriptional control sequence is operably connected to the nucleic acid molecule of the invention. For the purposes of the present specification, a transcriptional control sequence is regarded as “operably connected” to a given nucleic acid molecule when the transcriptional control sequence is able to promote, inhibit or otherwise modulate the transcription of the nucleic acid molecule. Therefore, in some embodiments, the nucleic acid molecule is under the control of a transcription control sequence, such as a constitutive promoter or an inducible promoter.

The “nucleic acid construct” may be in any suitable form, such as in the form of a plasmid, phage, transposon, cosmid, chromosome, vector, etc., which is capable of replication when associated with the proper control elements and which can transfer gene sequences, contained within the construct, between cells. Thus, the term includes cloning and expression vehicles, as well as viral vectors. In some embodiments, the nucleic acid construct is a vector. In some embodiments the vector is a viral vector.

A promoter may regulate the expression of an operably connected nucleic acid molecule constitutively, or differentially, with respect to the cell, tissue, or organ at which expression occurs. As such, the promoter may include, for example, a constitutive promoter, or an inducible promoter. A “constitutive promoter” is a promoter that is active under most environmental and physiological conditions. An “inducible promoter” is a promoter that is active under specific environmental or physiological conditions. The present invention contemplates the use of any promoter which is active in a cell of interest. As such, a wide array of promoters would be readily ascertained by one of ordinary skill in the art.

Mammalian constitutive promoters may include, but are not limited to, Simian virus 40 (SV40), cytomegalovirus (CMV), P-actin, Ubiquitin C (UBC), elongation factor-1 alpha (EF1A), phosphoglycerate kinase (PGK) and CMV early enhancer/chicken β actin (CAGG).

Inducible promoters may include, but are not limited to, chemically inducible promoters and physically inducible promoters. Chemically inducible promoters include promoters which have activity that is regulated by chemical compounds such as alcohols, antibiotics, steroids, metal ions or other compounds. Examples of chemically inducible promoters include: tetracycline regulated promoters (e.g. see U.S. Pat. Nos. 5,851,796 and 5,464,758); steroid responsive promoters such as glucocorticoid receptor promoters (e.g. see U.S. Pat. No. 5,512,483), ecdysone receptor promoters (e.g. see U.S. Pat. No. 6,379,945) and the like; and metal-responsive promoters such as metallothionein promoters (e.g. see U.S. Pat. Nos. 4,940,661, 4,579,821 and 4,601,978) amongst others.

As mentioned above, the control sequences may also include a terminator. The term “terminator” refers to a DNA sequence at the end of a transcriptional unit which signals termination of transcription. Terminators are 3-non-translated DNA sequences generally containing a polyadenylation signal, which facilitate the addition of polyadenylate sequences to the 3′-end of a primary transcript. As with promoter sequences, the terminator may be any terminator sequence which is operable in the cells, tissues or organs in which it is intended to be used. Suitable terminators would be known to a person skilled in the art.

As will be understood, the nucleic acid construct can further include additional sequences, for example sequences that permit enhanced expression, cytoplasmic or membrane transportation, and location signals. Specific non-limiting examples include an Internal Ribosome Entry Site (IRES).

The present invention extends to all genetic constructs essentially as described herein. These constructs may further include nucleotide sequences intended for the maintenance and/or replication of the genetic construct in eukaryotes and/or the integration of the genetic construct or a part thereof into the genome of a eukaryotic cell.

Methods are known in the art for the deliberate introduction (transfection/transduction) of exogenous genetic material, such as the nucleic acid, into eukaryotic cells. As will be understood the method best suited for introducing the nucleic acid construct into the desired host cell is dependent on many factors, such as the size of the nucleic acid construct, the type of host cell the desired rate of efficiency of the transfection/transduction and the final desired, or required, viability of the transfected/transduced cells. Non-limiting examples of such methods include; chemical transfection with chemicals such as cationic polymers, calcium phosphate, or structures such as liposomes and dendrimers; non-chemical methods such as electroporation, sonoporations, heat-shock or optical transfection; particle-based methods such as ‘gene gun’ delivery, magnetofection, or impalefection or viral transduction.

The nucleic acid construct will be selected depending on the desired method of transfection/transduction. In some embodiments, the nucleic acid construct is a viral vector, and the method for introducing the nucleic acid construct into a host cell is viral transduction. Methods are known in the art for utilising viral transduction to elicit expression of a CAR in a PBMC (Parker, L L. et al. Hum Gene Ther. 2000;11: 2377-87) and more generally utilising retroviral systems for transduction of mammalian cells (Cepko, C. and Pear, W. Curr Protoc Mol Biol. 2001, unit 9.9). In other embodiments, the nucleic acid construct is a plasmid, a cosmid, an artificial chromosome or the like, and can be transfected into the cell by any suitable method known in the art.

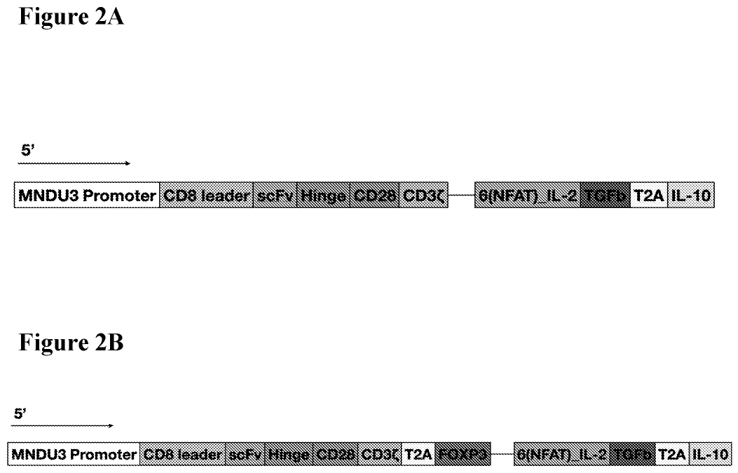

In addition to the domains and regions disclosed herein, the nucleic acid sequences as shown herein may also comprise promoter sequences specifically intended for the expression of the CAR construct, along with any other sequences that may be or are required for functional expression of the CAR constructs disclosed herein. In one example, the nucleic acid sequence for expression of the CAR construct comprises a promoter at its 5′ or N terminus. In another example, the sequence of expression of a CAR construct comprises a leader sequence (may also be referred to as a signal peptide), whereby the leader sequence is operably linked to the promoter sequence. In one example, the promoter is MNDU3. In another example, the leader sequence is a CD8 leader sequence. In one example, the sequence for expression comprises both a MNDU3 promoter and a CD8 leader sequence. Examples sequences of expression of a CAR construct can be found in, for example, FIGS. 2A and 2B. The promoter and leader sequences as disclosed herein are thus inserted or present upstream of the sequences encoding the extracellular domains of the CAR constructs disclosed here and are operably linked to the same.

In another aspect, the present disclosure refers to a first polynucleotide encoding the chimeric antigen receptor as disclosed herein.

In another aspect, the present disclosure also refers to a vector comprising the first polynucleotide as disclosed herein. In a specific example, the vector is a viral vector, optionally a lentiviral vector.

### Modified Cells

In another aspect of the present disclosure, there is provided a modified cell, preferably a modified T cell, comprising the chimeric antigen receptor as disclosed herein, the first polynucleotide, nucleic acid or nucleic acid construct as disclosed herein, and/or the vector as disclosed herein.

The cell to be genetically modified can be obtained from any suitable source. In some embodiments of any aspect of the present invention the cell to be genetically modified is an autologous cell, being a cell autologous to the subject to be treated. Advantageously, an autologous cell would not be recognised as ‘non-self by the subject’s immune system and would therefore be tolerated by the subject. However, autologous cells may not be readily available. Therefore, in some embodiments of the invention the cell to be genetically modified is an allogeneic or heterologous cell.

In one example, the modified T cell of the present disclosure is modified from a regulatory T cell (Treg). In this example, the Treg is isolated from a subject, or is isolated from a subject and expanded ex vivo. In an example, the Treg is modified with the transfection of the one or more vectors of the present disclosure after it is isolated and optionally expanded ex vivo. In another example, the modified T cell is derived from an induced pluripotent stem cell (iPSC). In a specific example, the iPSC is modified with the transfection of the one or more vectors of the present disclosure to arrive at the modified T cell of the present disclosure. In a preferred example, the iPSC is derived from a donor T cell.

Upon creation of a genetically modified cell in accordance with the present disclosure it may be desirable to expand the cell population in vitro to increase the total cell numbers available for use in treatment. This can be done using the step of exposing the cell to an antigen for the CAR. Accordingly, the present disclosure also provides a method of expanding in vitro the genetically modified cell as described herein, the method including the step of exposing the cell to an antigen for the CAR. In some embodiments, the method includes the further step of exposing the cell to a cytokine. For example, the present disclosure also includes a method of expanding in vitro the genetically modified cell, the method including the step of exposing the cell to an antigen for the CAR and simultaneously exposing the cell to a cytokine.

In another aspect, the present disclosure provides a method of expanding in vitro the genetically modified cell as described herein, the method including exposing the cell to immobilised anti-CD3 and anti-CD28 antibodies. In some embodiments, the antibodies are immobilised on a beaded substrate (for example “Human Activator” Dynabeads™). In some embodiments, the antibodies are immobilised on an alternative surface such as the surface of a tissue culture vessel, a culture flask, plate or bioreactor.

As would be understood by a person skilled in the art, depending on the signalling domain of the CAR, recognition by the CAR of its cognate antigen will lead to intracellular signalling that may ultimately lead to cellular proliferation. Accordingly, small numbers of cells, or even individual cells, can be expanded (or in the case of a single cell, clonally expanded) to form therapeutically significant numbers. This process can be further enhanced by the provision of cytokines.

The delivery or administration of the genetically modified cell may be delivery or administration of the cell alone, or delivery or administration of the cell formulated into a suitable pharmaceutical composition. Accordingly, the present invention provides a pharmaceutical composition including a genetically modified cell, and a pharmaceutically acceptable carrier.

Methods are known in the art for providing CAR-containing cells for immunotherapy (see for example Kershaw, M H. et al. Clin Cancer Res. 2006; 12(20): 6106-15; Parker L L. et al. Hum Gene Ther 2000;11: 2337-87). Furthermore, protocols and methods are known in the art for the preparation, expansion and assessment of mammalian CAR-expressing cells (see for example Cheadle, E J. et al. Antibody Engineering: Methods and Protocols, Second Edition, Methods in Molecular Biology, vol. 907: 645-66) and are summarised in the Examples below.

The pharmaceutical composition may also include one or more pharmaceutically acceptable additives, including pharmaceutically acceptable salts, amino acids, polypeptides, polymers, solvents, buffers, excipients and bulking agents, taking into consideration the particular physical and chemical characteristics of the cell to be administered. In some embodiments, the pharmaceutical composition includes a suspension of genetically modified cells in a suitable medium, such as isotonic saline solution. In some embodiments, the pharmaceutical composition may include suitable adjuvants such as one or more cytokines as described above.

Administration of the pharmaceutical composition may also be via parenteral means which include intravenous, intraventricular, intraperitoneal, intramuscular or intracranial injection, or local injections to the site of a tumour or cancerous mass.

### Immunosuppressive Molecules and Inducible System

In an example, the modified T cell expresses one or more immunosuppressive molecules. The expression can be constitutive expression or inducible expression. In a specific example, the one or more immunosuppressive molecules are expressed when the modified T cell is activated. In certain examples, the T cell is activated by the chimeric antigen receptor disclosed herein and expressed by the modified T cell, specifically through its signalling domain.

In a specific example, the inducible expression is enabled by a NFAT-inducible system. In a specific example, the modified T cell comprises a second polynucleotide encoding a promoter comprising one or more binding sites for NFAT (nuclear factor of activated T cells), and one or more immunosuppressive molecules; wherein the binding of NFAT to the promoter induces the expression of the one or more immune-suppressive molecules. In one example, both the first and the second polynucleotide, as disclosed herein, are expressed from one nucleic acid expression sequence. It is known in the technical field that NFAT is a major response to T cell activation (via CD3 zeta and CD28 signalling) and hence the molecule of choice for coupling the expression of the immunosuppressors with the activation of the chimeric antigen receptor as disclosed herein. Such immunosuppressors are, but are not limited to, IL-10, TGF-beta, FOXP3 and combinations thereof. Nevertheless, factors like NF-κB could be potentially induced by many other non-CD3zeta dependent ways, and the chimeric antigen receptor could be modified accordingly. In a more specific example, the promotor comprises, or is, the promoter of Interleukin-2 (IL-2), or a derivative thereof. NFAT motifs in the IL2 promoter is well characterised and hence is a preferred example for the promoter. It is known generally in the art that the number of NFAT binding sites can dictate the level of activation. In a specific example, the promoter is a 6(NFAT) minimal IL-2 promoter which comprises 6 NFAT binding sites. In an example where the second polynucleotide encodes more than one immunosuppressive molecule, the coding sequences of the immunosuppressive molecules can be separated by one or more linker sequences. In certain examples, the one or more linker sequences encode glycine-senne (GS) linkers, for example GSG linkers. In a further example, each of the one or more linker sequences further encodes a self-cleaving peptide, preferably a T2A peptide. In an example, the one or more immunosuppressive molecules are capable of suppressing the activity of effecter T cells or are capable of inducing anergy in effector T cells. In a specific example, the one or more immunosuppressive molecules are selected from the group consisting of IL-10, TGF-beta, CTLA-4, LAG3, PD-L1 and PD-1. In a specific example, the one or more immunosuppressive molecules is/are IL-10 and/or TGF-beta. In a specific example, the second polynucleotide comprises the sequence as set forth in SEQ ID: 10. It would be immediately apparent to a person skilled in the art that the first, second and/or third polynucleotides can be encoded on one, two, three or more vectors, and in any combination. For example, the first polynucleotide which comprises the CAR construct could further comprise the second or third polynucleotide, or both. It is also conceivable for the second polynucleotide to be broken down into several polynucleotide segments encoded on different vectors, wherein each polynucleotide segment is responsible for expressing a different immunosuppressor.

With regard to the nucleic acid sequences encoding the inducible cassette, as described above, the sequences encoding each part of the CAR construct can be found on one or more (nucleic acid) expression sequences. In one example, the nucleic acid sequences for expressing the promoter, the leader sequence, the extracellular sequence, the transmembrane domain, the intracellular domain and the inducible cassette, as described herein, are found on one expression sequence. In another example, the nucleic acid sequences for expressing the promoter, the leader sequence, the extracellular sequence, the transmembrane domain, the intracellular domain and the inducible cassette, as described herein, are found on separate expression sequences, whereby the separate expression sequences are operably linked to each other in a manner that enables expression of a functional CAR construct.

### Inducible Suicide System

In a further example, the modified T cell further comprises a third polynucleotide, said third polynucleotide encoding an inducible suicide system, wherein contacting of an inducer molecule by the modified T cell will induce cell death in said modified T cell. An example for the inducible suicide system is a caspase-9 (iCasp9) suicide gene system. In this system, the inducer is CID (also known as AP1903/Rimiducid). Examples of the iCasp9 based inducible suicide system are known in the art and are described in the following non-exhaustive list of publications: An inducible caspase 9 safety switch can halt cell therapy-induced autoimmune disease (de Witte M A, Jorritsma A, Swart E, Straathof K C, de Punder K, Haanen J B, Rooney C M, Schumacher T N; J Immunol. 2008 May 1; 180(9):6365-73); Combining a CD20 chimeric antigen receptor and an inducible caspase 9 suicide switch to improve the efficacy and safety of T cell adoptive immunotherapy for lymphoma (Budde L E, Berger C, Lin Y, Wang J, Lin X, Frayo S E, Brouns S A, Spencer D M, Till B G, Jensen M C, Riddell S R, Press O W PLoS One. 2013; 8(12):e82742).

### Hypoimmunogenic Modified Cells

In an example, the modified T cell as disclosed herein is hypoimmunogenic. In a specific example, the modified T cell is genetically edited to become hypoimmunogenic. In a specific example, one or more genes of the Major Histocompatibility Class (MHC) I, and one or more genes of the Major Histocompatibility Class (MHC) II are deleted or mutated, wherein the deletion of the mutations of the genes result in the dysfunction of the MHC I and MHC II complexes. In a more specific example, the one or more genes of MHC I and II comprise at least B2M (B2 microglobulin) and CIITA (class II MHC transactivator). In a specific example, further editing can be introduced to the modified T cell to increase its hypoimmunogenicity. In an example, the modified T cell is further modified to overexpress CD47 (Cluster of Differentiation 47) or a functional variant thereof. The overexpression of CD47 on the cell surface could further prevent recognition and clearance of the modified T cell by a non-self-host immune system. It is to be understood that the above-mentioned genetic editing could be applied to modified T cells that are either iPSC-derived or Treg-derived. The hypoimmunogenic modified T cell can serve as “stealth” cells which are not targeted by the immune system when administered to an individual. The hypoimmunogenic modified T cells are suitable for allogenic administration, wherein the cells do not have to be derived from or histocompatible with the individual receiving the administration.

### Methods of Generating Genetically Modified Cells

In another aspect of the present disclosure, there is provided a method of generating Chimeric antigen receptor (CAR)-regulatory T cells (CAR-Tregs), said method comprising transducing Tregs with the vector as disclosed herein, so that the transduced Tregs express the CAR of the present disclosure, thereby generating Chimeric antigen receptor (CAR)-regulatory T cells (CAR-Tregs).

In another aspect of the present disclosure, there is provided a method of generating Chimeric antigen receptor (CAR)-regulatory T cells (CAR-Tregs), said method comprising: a) isolating regulatory T cells (Treg) from peripheral blood mononuclear cells (PBMCs) from a subject; b) expanding the isolated Tregs ex vivo; and c) transducing the expanded Tregs with the vector as disclosed herein, so that the transduced Tregs express the CAR of the present disclosure.

In another aspect of the present disclosure, there is provided a method of generating modified a hypoimmunogenic T cell, said method comprising: a) modifying a CD34+ iPSC to (i) reduce or eliminate the level of expression or activity of B2 microglobulin and class II MHC transactivator, and (ii) overexpress CD47 or a functional variant thereof; b) transducing the modified CD34+ iPSC with a vector of the present disclosure, so that the transduced CD34+ iPSC expresses the CAR of the present disclosure; c) differentiating the CAR hypoimmunogenic iPSC to an iPSC-derived T cell. Preferably the iPSC-derived T cell is an iPSC-derived T-regulatory (Treg) cell.

In another aspect of the present disclosure, there is provided a method of generating modified a hypoimmunogenic T cell, said method comprising: a) providing a CD34+ iPSC, b) reducing or eliminating the level of expression of activity of B2 microglobulin and class II MHC transactivator in the CD34+ iPSC; c) overexpressing CD47 or a functional variant thereof in the CD34+ iPSC of b); d) transducing the CD34+ iPSC of c) with a vector of the present disclosure, so that the transduced CD34+ iPSC expresses the CAR of the present disclosure; e) differentiating the CAR hypoimmunogenic iPSC to an iPSC-derived T cell. Preferably the iPSC-derived T cell is an iPSC-derived T-regulatory (Treg) cell.

In another aspect of the present disclosure, there is provided a method of generating modified hypoimmunogenic T cells, said method comprising: a) editing the genome of CD34+ iPSCs to reduce or eliminate the expression of functional gene products of B2M (B2 microglobulin) and CIITA (class II MHC transactivator) genes; c) over expressing CD47 or a functional variant thereof in the CD34+ iPSCs; d) transducing the CD34+ iPSCs of c) with the vector of the present disclosure, so that the transduced cells express the CAR of the present disclosure; e) differentiating the CAR hypoimmunogenic iPSCs to iPSC-derived T cells. Preferably the iPSC-derived T cells are iPSC-derived T-regulatory (Treg) cells.

In another aspect of the present disclosure, there is provided a method of generating modified hypoimmunogenic T cells, said method comprising: a) CD34+ induced pluripotent stem cells (iPSCs); b) editing the genome of the iPSCs to knockout both B2M (B2 microglobulin) and CIITA (class II MHC transactivator) genes; c) gene editing iPSCs from b) with a template encoding CD47 or a functional variant thereof, wherein the knocked in iPSC overexpresses CD47; d) transducing the gene edited iPSCs with the vector of the present disclosure, so that the transduced iPSCs express the chimeric antigen receptor of the present disclosure; e) differentiating the gene edited CAR-iPSCs to iPSC-derived T cells.

In a specific example, steps b) and c), or any editing of a genome as described herein, are carried out using the CRISPR-Cas gene editing system. The methods of utilising CRISPR-Cas gene editing is well known in the art and widely used, with commercial kits readily available.

The method for differentiating iPSCs to iPSC-derived T cells are also well-known in the art with commercial kits available. A detailed example for this method can be found in Kaneko S. (2016), In Vitro Generation of Antigen-Specific T Cells from Induced Pluripotent Stem Cells of Antigen-Specific T Cell Origin. In: Bondanza A., Casucci M. (eds) Tumor Immunology. Methods in Molecular Biology, vol 1393. Humana Press, New York, NY. DOI: 10.1007/978-1-4939-3338-9_6.

In a further example, the method further comprises any one or more ofthe following: g) testing for the expression of the chimeric antigen receptor of the present disclosure; h) testing the functional potential of the chimeric antigen receptor-Tregs or modified T cells to produce one or more immunosuppressive molecules in response to contacting the inflammation associated factors; and i) testing the functional potential of the chimeric antigen receptor-Tregs or modified T cells to suppress activation/proliferation of effector T cells.

In specific examples of the methods as described above, the vector is a viral vector, preferably a lentiviral vector. Viral vectors of the disclosure may comprise a sequence isolated or derived from a retrovirus, a lentivirus, an adenovirus, an adeno-associated virus or any combination thereof. The viral vector may comprise a sequence isolated or derived from an adeno-associated virus (AAV). The viral vector may comprise a recombinant AAV (rAAV). Exemplary adeno-associated viruses and recombinant adeno-associated viruses of the disclosure comprise two or more inverted terminal repeat (ITR) sequences located in cis next to a sequence encoding a CAR of the disclosure. Exemplary adeno-associated viruses and recombinant adeno-associated viruses of the disclosure include, but are not limited to all serotypes (e.g. AAV1, AAV2, AAV3, AAV4, AAV5, AAV6, AAV7, AAV8, and AAV9). Exemplary adeno-associated viruses and recombinant adeno-associated viruses of the disclosure include, but are not limited to, self-complementary AAV (scAAV) and AAV hybrids containing the genome of one serotype and the capsid of another serotype (e.g. AAV2/5, AAV-DJ and AAV-DJ8).

### Methods of Therapy

In yet another aspect, the present disclosure refers to a modified cell, preferably a modified T cell, of the present disclosure for use in therapy.

Any disease/condition involving or resulting from uncontrolled inflammatory responses mediated by one or more inflammation associated factor(s), for example disclosed herein, can be treated using a modified cell (for example, modified T cell, or CAR Treg cell) or nucleic acid as disclosed herein. Typically, the inflammation associated fact is TNF alpha and/or IFN gamma. The disease/condition includes but is not limited to inflammatory autoimmune diseases, transplantation, cytokine release syndrome and any other disease or condition described herein.

In any aspect or embodiment, the present disclosure includes a method of treating a disease or condition where disease or condition pathogenesis involves TNF alpha or IFN gamma.

In yet another aspect, the present disclosure refers to a modified cell (for example, modified T cell, or CAR Treg cell) or nucleic acid of the present disclosure for use in the treatment of an autoimmune disease; a transplant rejection, or a graft versus host disease (GVHD).

In yet another aspect, the present disclosure refers to a method of treating an autoimmune disease, a transplant rejection, or a graft versus host disease (GVHD), wherein the method comprises administering a therapeutically effective number of the modified cells, preferably modified T cells, or amount of nucleic acid as disclosed herein.

In an example, the autoimmune disease to be treated is selected from the group consisting of rheumatoid arthritis, psoriatic arthritis, psoriasis, lupus, juvenile rheumatoid arthritis, multiple sclerosis, inflammatory bowel disease, and Crohn's disease. In another example, the transplant rejection to be treated is selected from the group consisting of organ transplant rejection, stem cell transplant rejection, and bone marrow transplant rejection.

In still another aspect, the present disclosure refers to a method of inducing immune tolerance in a subject in need thereof, wherein the method comprises administering a therapeutically effective number of the modified cells, preferably modified T cells, or amount of nucleic acid as disclosed herein.

In still another aspect, the present disclosure refers to a method of downregulating inflammation locally or systemically in a subject in need thereof, wherein the method comprises administering a therapeutically effective number of the modified cells, preferably modified T cells, or amount of nucleic acid as disclosed herein.

In still another aspect, the present disclosure refers to a method of suppressing the activity of effecter T cells locally or systematically in a subject, wherein the method comprises administering a modified cell, preferably a modified T cell, or nucleic acid of the present disclosure to the subject locally or systematically.

In another aspect of the present disclosure, there is provided a vector comprising a sequence as provided in the present disclosure.

T Cell Isolation from a Leukapheresis Product

A leukapheresis product or blood may be collected from a subject at clinical site using a closed system and standard methods (e.g., a COBE Spectra Apheresis System). Preferably, the product is collected according to standard hospital or institutional Leukapheresis procedures in standard Leukapheresis collection bags. For example, in preferred embodiments of the methods of the disclosure, no additional anticoagulants or blood additives (heparin, etc.) are included beyond those normally used during leukapheresis.

Alternatively, white blood cells (WBC)/Peripheral Blood Mononuclear Cells (PBMC) (using Biosafe Sepax 2 (Closed/Automated)) or T cells (using CliniMACS® Prodigy (Closed/Automated)) may be isolated directly from whole blood. However, in certain subjects (e.g. those diagnosed and/or treated for cancer), the WBC/PBMC yield may be significantly lower when isolated from whole blood than when isolated by leukapheresis.

Either the leukapheresis procedure and/or the direct cell isolation procedure may be used for any subject of the disclosure.

The leukapheresis product, blood, WBC/PBMC composition and/or T-cell composition should be packed in insulated containers and should be kept at controlled room temperature (+19° C. to +25° C.) according to standard hospital of institutional blood collection procedures approved for use with the clinical protocol. The leukapheresis product, blood, WBC/PBMC composition and/or T-cell composition should not be refrigerated.

The cell concentration leukapheresis product, blood, WBC/PBMC composition and/or T-cell composition should not exceed 0.2×109 cells per mL during transportation. Intense mixing of the leukapheresis product, blood, WBC/PBMC composition and/or T-cell composition should be avoided.

If the leukapheresis product, blood, WBC/PBMC composition and/or T-cell composition has to be stored, e.g. overnight, it should be kept at controlled room temperature (same as above). During storage, the concentration of the leukapheresis product, blood, WBC/PBMC composition and/or T-cell composition should never exceed 0.2×109 cell per mL.

Preferably, cells of the leukapheresis product, blood, WBC/PBMC composition and/or T-cell composition should be stored in autologous plasma. In certain embodiments, if the cell concentration of the leukapheresis product, blood, WBC/PBMC composition and/or T-cell composition is higher than 0.2×109 cell per mL, the product should be diluted with autologous plasma.

Preferably, the leukapheresis product, blood, WBC/PBMC composition and/or T-cell composition should not be older than 24 hours when starting the labeling and separation procedure. The leukapheresis product, blood, WBC/PBMC composition and/or T-cell composition may be processed and/or prepared for cell labeling using a closed and/or automated system (e.g., CliniMACS Prodigy).

An automated system may perform additional buffy coat isolation, possibly by ficolation, and/or washing of the cellular product (e.g., the leukapheresis product, blood, WBC/PBMC composition and/or T cell composition).

A closed and/or automated system may be used to prepare and label cells for T-Cell isolation (from, for example, the leukapheresis product, blood, WBC/PBMC composition and/or T cell composition).

Although WBC/PBMCs may be nucleofected directly (which is easier and saves additional steps), the methods of the disclosure may include first isolating T cells prior to nucleofection

T cells may be isolated directly, by enrichment of labeled cells or depletion of labeled cells in a one-way labeling procedure or, indirectly, in a two-step labeling procedure. According to certain enrichment strategies of the disclosure, T cells may be collected in a Cell Collection Bag and the non-labeled cells (non-target cells) in a Negative Fraction Bag. In contrast to an enrichment strategy of the disclosure, the non-labeled cells (target cells) are collected in a Cell Collection Bag and the labeled cells (non-target cells) are collected in a Negative Fraction Bag or in the Non-Target Cell Bag, respectively. Selection reagents may include, but are not limited to, antibody-coated beads. Antibody-coated beads may either be removed prior to a modification and/or an expansion step, or, retained on the cells prior to a modification and/or an expansion step. One or more of the following non-limiting examples of cellular markers may be used to isolate T-cells: CD3, CD4, CD8, CD25, anti-biotin, CD1c, CD3/CD19, CD3/CD56, CD14, CD19, CD34, CD45RA, CD56, CD62L, CD133, CD137, CD271, CD304, IFN-gamma, TCR alpha/beta, and/or any combination thereof. Methods for the isolation of T-cells may include one or more reagents that specifically bind and/or detectably-label one or more of the following non-limiting examples of cellular markers may be used to isolate T-cells: CD3, CD4, CD8, CD25, anti-biotin, CD1c, CD3/CD19, CD3/CD56, CD14, CD19, CD34, CD45RA, CD56, CD62L, CD133, CD137, CD271, CD304, IFN-gamma, TCR alpha/beta, and/or any combination thereof. These reagents may or may not be “Good Manufacturing Practices” (“GMP”) grade. Reagents may include, but are not limited to, Thermo DynaBeads and Miltenyi CliniMACS products. Methods of isolating T-cells of the disclosure may include multiple iterations of labeling and/or isolation steps. At any point in the methods of isolating T-cells of the disclosure, unwanted cells and/or unwanted cell types may be depleted from a T cell product composition of the disclosure by positively or negatively selecting for the unwanted cells and/or unwanted cell types. A T cell product composition of the disclosure may contain additional cell types that may express CD4, CD8, and/or another T cell marker(s).

Methods of the disclosure for nucleofection of T cells may eliminate the step of T cell isolation by, for example, a process for nucleofection of T cells in a population or composition of WBC/PBMCs that, following nucleofection, includes an isolation step or a selective expansion step via TCR signaling.

Certain cell populations may be depleted by positive or negative selection before or after T cell enrichment and/or sorting. Examples of cell compositions that may be depleted from a cell product composition may include myeloid cells, CD8+ cytotoxic T cells, dendritic cells, macrophages, red blood cells, mast cells, gamma-delta T cells, natural killer (NK) cells, a Natural Killer (NK)-like cell (e.g. a Cytokine Induced Killer (CIK) cell), induced natural killer (iNK) T cells, NK T cells, B cells, or any combination thereof.

Preferred methods for T cell isolation may include a negative selection strategy for yielding untouched pan T cell, meaning that the resultant T-cell composition includes T-cells that have not been manipulated and that contain an endogenously-occurring variety/ratio of T-cells.

Reagents that may be used for positive or negative selection include, but are not limited to, magnetic cell separation beads. Magnetic cell separation beads may or may not be removed or depleted from selected populations of CD4+ T cells, CD8+ T cells, or a mixed population of both CD4+ and CD8+ T cells before performing the next step in a T-cell isolation method of the disclosure.

T cell compositions and T cell product compositions may be prepared for cryopreservation, storage in standard T Cell Culture Medium, and/or genetic modification.

T cell compositions, T cell product compositions, unstimulated T cell compositions, resting T cell compositions or any portion thereof may be cryopreserved using a standard cryopreservation method optimized for storing and recovering human cells with high recovery, viability, phenotype, and/or functional capacity. Commercially-available cryopreservation media and/or protocols may be used. Cryopreservation methods of the disclosure may include a DMSO free cryopreservant (e.g. CryoSOfree™ DMSO-free Cryopreservation Medium) reduce freezing-related toxicity.

T cell compositions, T cell product compositions, unstimulated T cell compositions, resting T cell compositions or any portion thereof may be stored in a culture medium. T cell culture media of the disclosure may be optimized for cell storage, cell genetic modification, cell phenotype and/or cell expansion. T cell culture media of the disclosure may include one or more antibiotics. Because the inclusion of an antibiotic within a cell culture media may decrease transfection efficiency and/or cell yield following genetic modification via nucleofection, the specific antibiotics (or combinations thereof) and their respective concentration(s) may be altered for optimal transfection efficiency and/or cell yield following genetic modification via nucleofection.

T cell culture media of the disclosure may include serum, and, moreover, the serum composition and concentration may be altered for optimal cell outcomes. Human AB serum is preferred over FBS/FCS for culture of T cells because, although contemplated for use in T cell culture media of the disclosure, FBS/FCS may introduce xeno-proteins. Serum may be isolated form the blood of the subject for whom the T-cell composition in culture is intended for administration, thus, a T cell culture medium of the disclosure may comprise autologous serum. Serum-free media or serum-substitute may also be used in T-cell culture media of the disclosure. In certain embodiments of the T-cell culture media and methods of the disclosure, serum-free media or serum-substitute may provide advantages over supplementing the medium with xeno-serum, including, but not limited to, healthier cells that have greater viability, nucleofect with higher efficiency, exhibit greater viability post-nucleofection, display a more desirable cell phenotype, and/or greater/faster expansion upon addition of expansion technologies.

T cell culture media may include a commercially-available cell growth media. Exemplary commercially-available cell growth media include, but are not limited to, PBS, HBSS, OptiMEM, DMEM, RPMI 1640, AIM-V, X—VIVO 15, CellGro DC Medium, CTS OpTimizer T Cell Expansion SFM, TexMACS Medium, PRIME-XV T Cell Expansion Medium, ImmunoCult-XF T Cell Expansion Medium, or any combination thereof.

T cell compositions, T cell product compositions, unstimulated T cell compositions, resting T cell compositions or any portion thereof may be prepared for genetic modification. Preparation of T cell compositions, T cell product compositions, unstimulated T cell compositions, resting T cell compositions or any portion thereof for genetic modification may include cell washing and/or resuspension in a desired nucleofection buffer. Cryopreserved T-cell compositions may be thawed and prepared for genetic modification by nucleofection. Cryopreserved cells may be thawed according to standard or known protocols. Thawing and preparation of cryopreserved cells may be optimized to yield cells that have greater viability, nucleofect with higher efficiency, exhibit greater viability post-nucleofection, display a more desirable cell phenotype, and/or greater/faster expansion upon addition of expansion technologies. For example, Grifols Albutein (25% human albumin) may be used in the thawing and/or preparation process.

### Infusion of Modified Cells as Adoptive Cell Therapy

The disclosure provides modified cells that express one or more CARs of the disclosure that have been selected and/or expanded for administration to a subject in need thereof. Modified cells of the disclosure may be formulated for storage at any temperature including room temperature and body temperature. Modified cells of the disclosure may be formulated for cryopreservation and subsequent thawing. Modified cells of the disclosure may be formulated in a pharmaceutically acceptable carrier for direct administration to a subject from sterile packaging. Modified cells ofthe disclosure may be formulated in a pharmaceutically acceptable carrier with an indicator of cell viability and/or CAR expression level to ensure a minimal level of cell function and CAR expression. Modified cells of the disclosure may be formulated in a pharmaceutically acceptable carrier at a prescribed density with one or more reagents to inhibit further expansion and/or prevent cell death.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used in this application, the singular form “a,” “an,” and “the” include plural references unless the context clearly dictates otherwise. For example, the term “a genetic marker” includes a plurality of genetic markers, including mixtures and combinations thereof.

As used herein, the term “about”, in the context of concentrations of components of the formulations, typically means+/−5% of the stated value, more typically +/−4% of the stated value, more typically +/−30% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Example 1—Synthesis of constructs

Synthesis of construct and cloning of constructs into lentiviral vectors are common and known techniques in the field of the invention. In the examples disclosed herein, the synthesis and viral packaging utilizes third generation lentivirus packaging plasmids consisting of 4 plasmids. Constructs are designed and cloned into the transfer plasmid containing the disclosed sequences.

### Example 2—Generation of CAR Treg Cells

Autologous chimeric antigen receptor (CAR) Treg cells can be generated by isolation and expansion of regulatory T cells (Tregs) from patients, which can then be used for treatment purposes. Regulatory T cells are isolated from peripheral blood mononuclear cells from healthy donors using commercially available immunomagnetic regulatory T cell isolation kit against CD25+ and CD127−CD4+ T cells (Stemcell Technologies) or using Fluorescent Activated Cell Sorting (FACS) against markers of CD4+, CD25+, CD127−CD14− cells. Purity of isolated cells is confirmed using flow cytometry by staining for CD3, CD4, CD25, FOXP3, CD127 (markers used for identifying regulatory T cells). Isolated Tregs are expanded using CD3/28 magnetic beads for 14 days.

### Example 3—Lentivirus-Based Transduction of Tregs and Validation

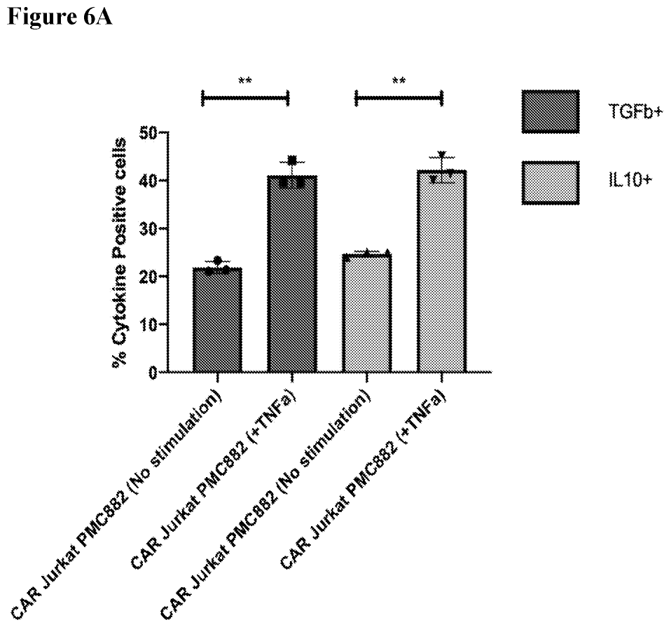

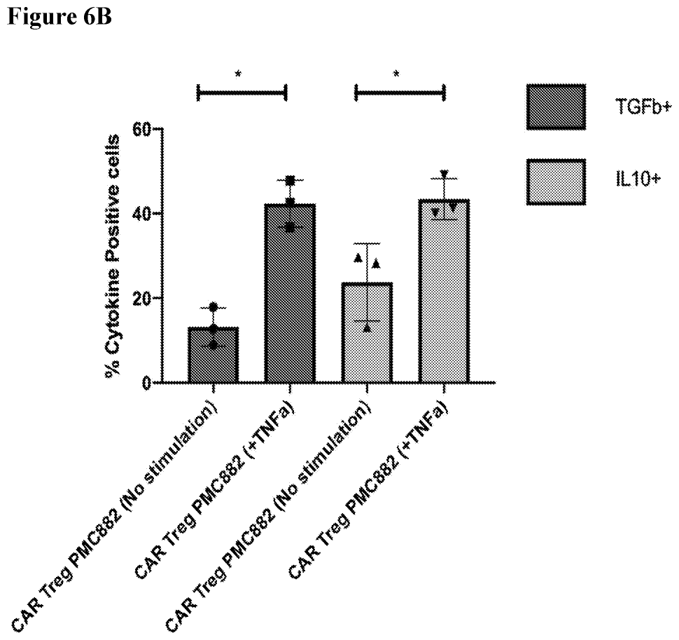

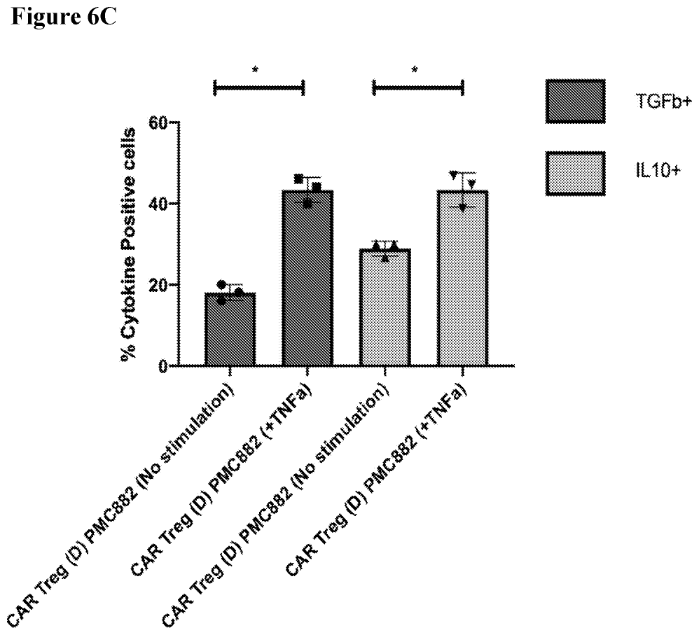

Isolated Tregs are transduced with lentivirus vectors containing the disclosed chimeric antigen receptor (CAR) sequences at one day post-activation with CD3/28 beads in the presence of polybrene (8 μg/ml; Sigma-Aldrich) or GMP-grade transduction activators (LentiBOOST; Sirion Biotech). At the end of 14 days of expansion, CAR Tregs are validated for cytokine production by transduced cells in response to recombinant TNF alpha and IFN gamma by ELISA and flow cytometry (FIG. 6). In addition, suppressive potential of CAR Treg cells is evaluated using activated T cell suppression assay.

### Example 4—Generation of iPSC-Derived T Cells

To overcome the limitation of autologous treatment and offer the consistency of off-the-shelf therapy, induced pluripotent stem cells (iPSCs) can be differentiated to become iPSC-derived T cells. In the first and preferred method, human CD34+ iPS cells from curated iPS cell bank sources such as Riken BioResource Research Center or ATCC. These CD34+ iPSCs can be differentiated to iPSC-derived T cells using commercially available kits such as STEMdiffr™ Cell Kit (Stemcell Technologies). Any disease/condition involving or resulting from uncontrolled inflammatory responses mediated via TNF alpha and/or IFN gamma can potentially be treated using the CAR Treg cells disclosed herein. These include but are not limited to inflammatory autoimmune diseases, transplantation, etc. Furthermore, the off-the-shelf iPSC-derived CAR Treg cells, as provided herein, negates the requirement of autologous T cells for CAR-T generation.

### Example 5—Gene-Editing of iPSCs

Firstly, CD34+ iPSCs undergo 2 rounds of gene modification to be made hypoimmunogenic. The first round involves CRISPR-Cas based knock out of B2M and CIITA genes. Commercially available kits such as IDT Alt-R CRISPR systems are used as per manufacturer's protocol. Briefly, gRNA is synthesised (B2M guide RNA: GAGTAGCGCGAGCACAGCTA; CIITA guide RNA TCCAGGTAGCCACCTTCTAG) and added with the optimized recombinant cas9 protein to form the ribonucleoprotein (RNP) which is transfected into the cell via electroporation or liposomes. After transfection, single cell clones are isolated and expanded into colonies. Colonies are screened for successful CRISPR editing by sequencing for gene disruption as well as ensuring no off-target effects. In addition, cells are tested for pluripotency and normal karyotype. A second round of gene editing involves CRISPR-cas9 induced homology directed repair for site-specific integration of the overexpressing CD47 template.

The successfully CRISPR-edited hypoimmunogenic iPSCs are then lentivirally transduced with the CAR sequences disclosed here. iPSCs that successfully integrates and expresses the CAR construct are subsequently single cell cloned and banked.

### Example 6—Differentiation of Hypoimmunogenic CAR-iPSCs to CAR-iPSC-Derived T Cells

The hypoimmunogenic CAR-iPSCs are cultured with feeder cells in EB medium (Embryoid Body Formation Medium) containing rhVEGF (recombinant human Vascular endothelial growth factor for 7 days, followed by addition of rhCSF (recombinant human Colony-stimulating factor-1) and rhFlt-3L (recombinant human Fins-related tyrosine kinase 3 ligand). On day 14, hematopoietic progenitor cells are collected and transferred onto OP9-DL1 cells in the presence of rhIL7 (recombinant human Interleukin-7) and rhFlt-3L. T cell are harvested and stimulated in the presence of rhIL7 and dexamethasone. Other methods include using feeder free conditions such as STEMdiffr^{M} T Cell Kit (Stemcell Technologies) as well as method developed by Iriguchi, S., Yasui, Y., Kawai, Y., Arima, S., Kunitomo, M., Sato, T., & Kaneko, S. (2021). Feeder-free differentiation and expansion for T cells from induced pluripotent stem cells.

### Example 7—Hypoimmunogenic, iPSC Derived CAR-T Cells

A limitation in CAR-T cell-based therapy is availability of autologous cells for engineering. Development of “Off the shelf” allogeneic cells for cellular therapy is one of the big goals in CAR-T field. Disclosed herein is a method for modification of allogenic T cells to hypoimmunogenic CAR-T cells. Briefly, T cells from healthy donors are re-programmed into induced pluripotent stem cells or using curated CD34+ iPS for the ease of expansion and as a ready supply of cells. These iPSCs are made hypoimmunogenic by inactivation of MHC class I and II and over-expression of CD47. These cells are then differentiated to generate iPS-derived T cells, that can be modified further to become antigen-specific. This antigen specificity is the result of introduction of a chimeric antigen receptor (CAR) along with the expression of FOXP3, a master transcription factor for regulatory T cells, to inherit regulatory functions.

### Example 8—Construction of Inflammatory Soluble Factor Specific CARs

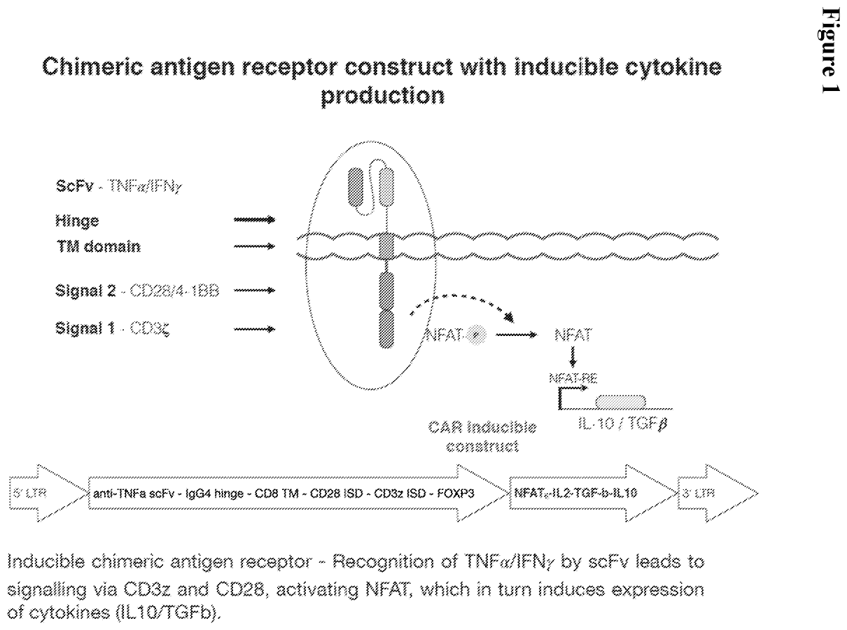

The CAR design for the inducible cassette, named PMC882, contains two parts (FIG. 2A). The first part is the CAR cassette contains the antigen recognition domain (scFv) with specificity to inflammatory soluble factors. Upon binding to these factors, the CAR co-stimulatory domain, CD28 and CD3zeta becomes phosphorylated, activating the cell (FIG. 1). The second part is the immunosuppressive cassette containing immunosuppressive cytokines TGF beta (Tumour Growth Factor Beta) and IL10 (interleukin 10). The inducible promotor NFAT (Nuclear Factor of Activated T-cells) lead to the expression of these cytokines only when the cell is activated.

Another design adds the FOXP3 (forkhead box P3), a transcription factor for regulatory T cells, with the CAR cassette, separated by a translational self-cleaving peptide sequence, T2A. (FIG. 2B).

### Example 9—Expression and Antigen Specificity of CAR Design

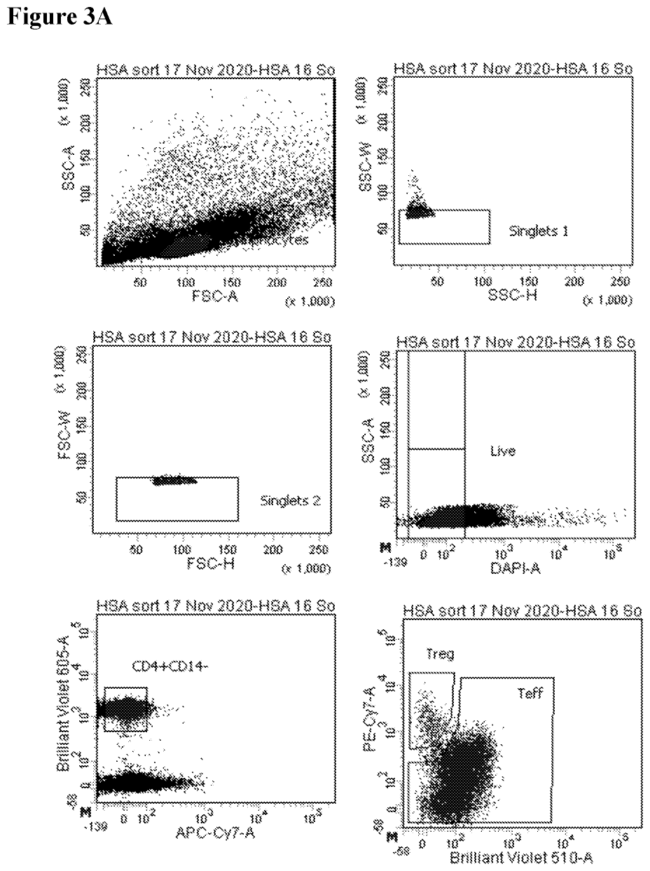

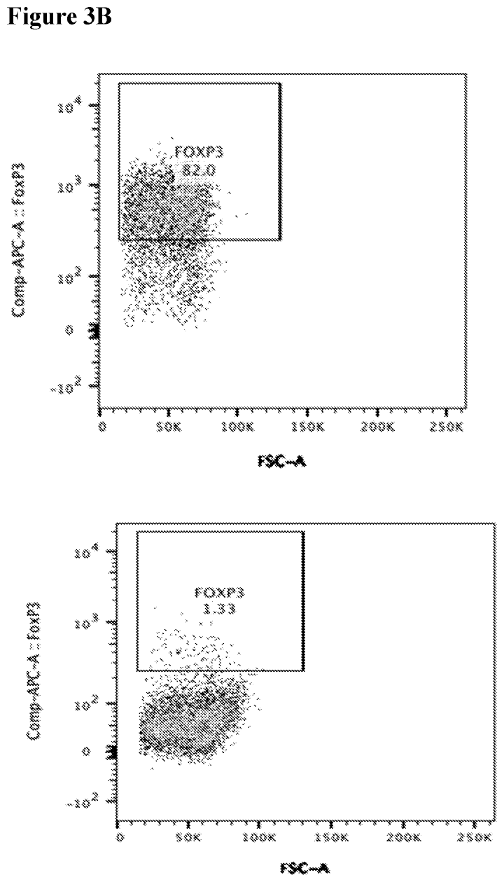

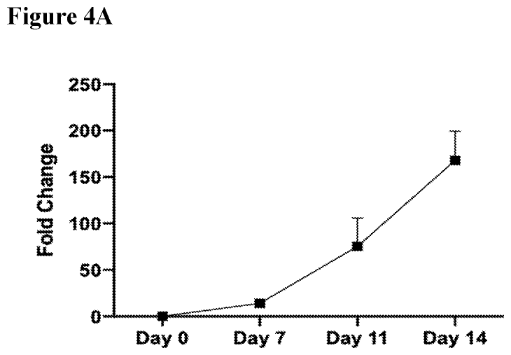

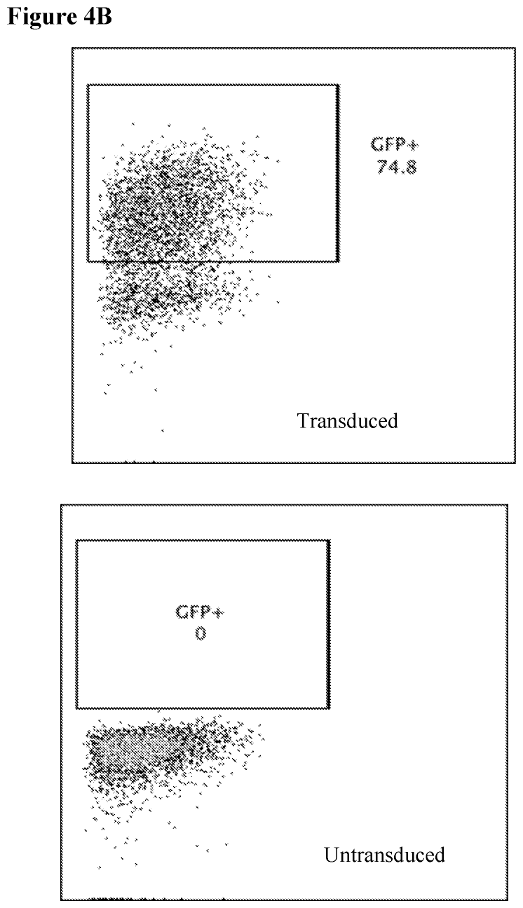

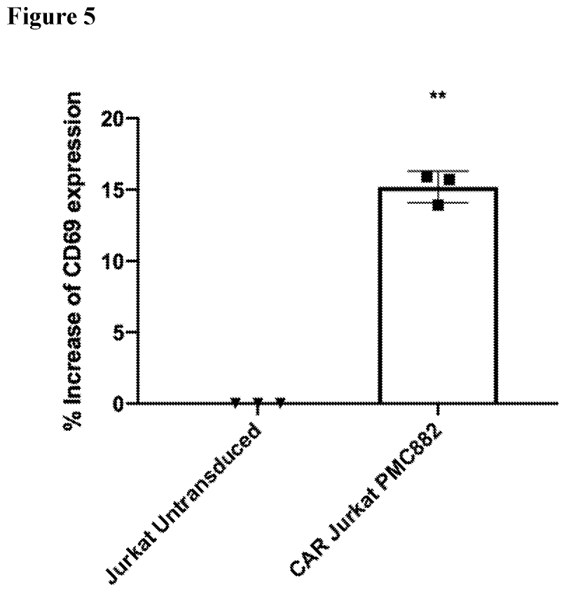

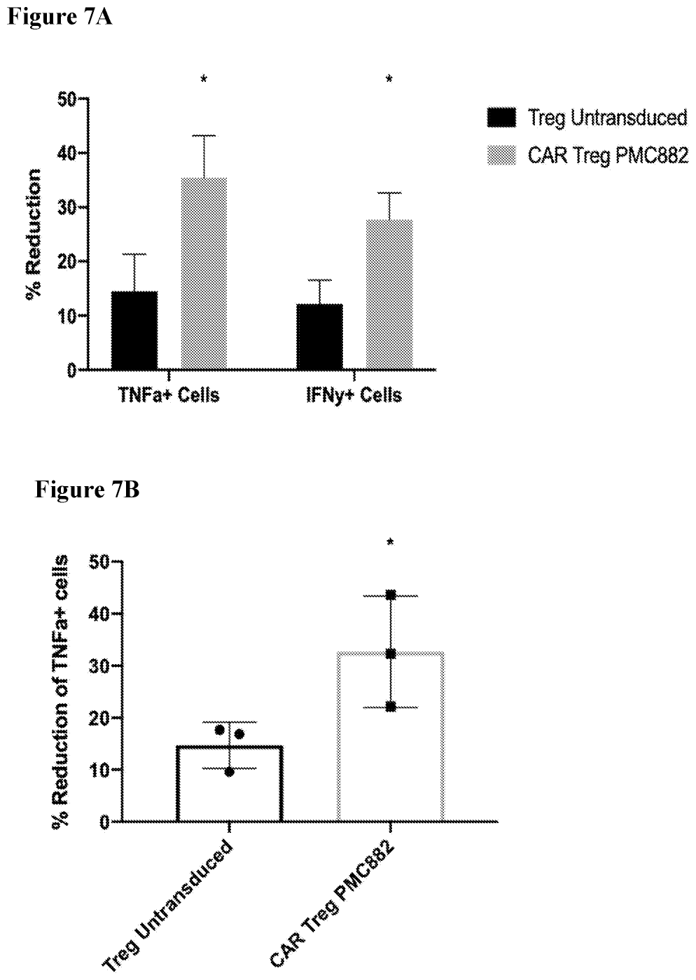

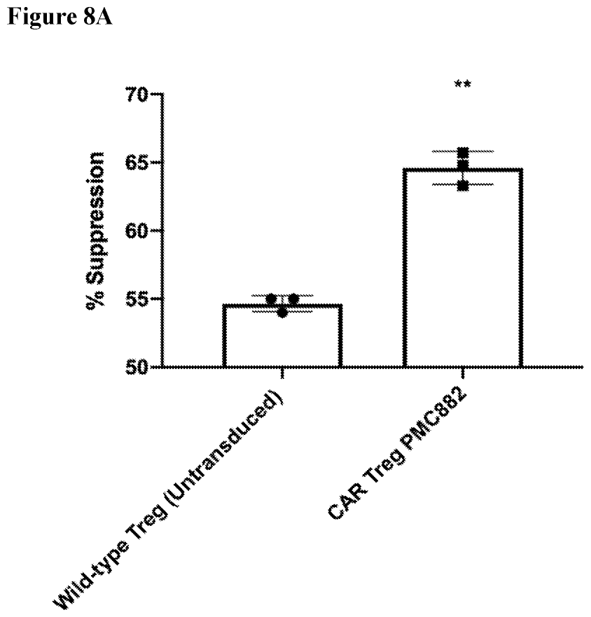

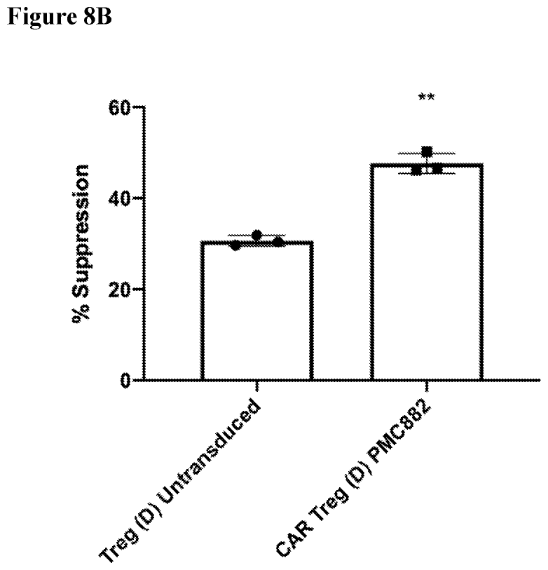

The CAR design disclosed herein is activated by inflammatory soluble factors, rather than surface bound antigens as with current and known CAR T cell therapies. In autoimmune diseases, these inflammatory soluble factors are pathogenic and play a role in disease progression. The design disclosed herein enables CAR Tregs to be activated at the site of inflammation and subsequently release immunosuppressive factors such as, but not limited to, TGF beta and IL10. Healthy or autoimmune-diseased (for example, but not limited to, rheumatoid arthritis) primary regulatory T cells (Tregs) were isolated using fluorescence-activated cell sorting (FACS) of peripheral blood mononuclear cells (PBMCs). PBMCs stained with CD4, CD14, CD25 and CD127 (FIG. 3A) were sorted at high purity for Tregs as indicated by Treg marker FOXP3 (FIG. 3B). Use of CD3/28 Dynabeads and lentiviral transduction of construct anti-TNFa scFv PMC882 of primary Tregs led to expansion of Tregs (FIG. 4A) and a high proportion of CARpositive Tregs (GFP+). PMC882 CAR Jurkat cells (FIG. 6A), PMC882 CAR Tregs from healthy donor (FIG. 6B) and PMC882 CAR Tregs from Juvenile Idiopathic Arthritic (JIA) active disease donors (FIG. 6C) showed an increase of TGF beta and IL10 positive cells in the presence of TNF alpha. Example 10—CAR Tregs superior to untransduced Tregs in suppression assay PMC882 CAR Tregs from both healthy (FIG. 7A) and active disease donors (FIG. 7B) were able to reduce autologous pro-inflammatory T cells based on CD3 and CD28 activation. In an autologous effector T cells suppression assay, PMC882 CAR Tregs increased suppression compared to untransduced Tregs from healthy (FIG. 8A) and active disease (FIG. 8B) donors (suppression being suppressing the expansion of activated effector T cells). This demonstrates the function of CAR Tregs with the immunosuppressive cassette of TGF beta and IL10 in suppressing activated effector T cells.

### Example 11—CAR Tregs Increases Survival in NSG GvHD Mouse Model

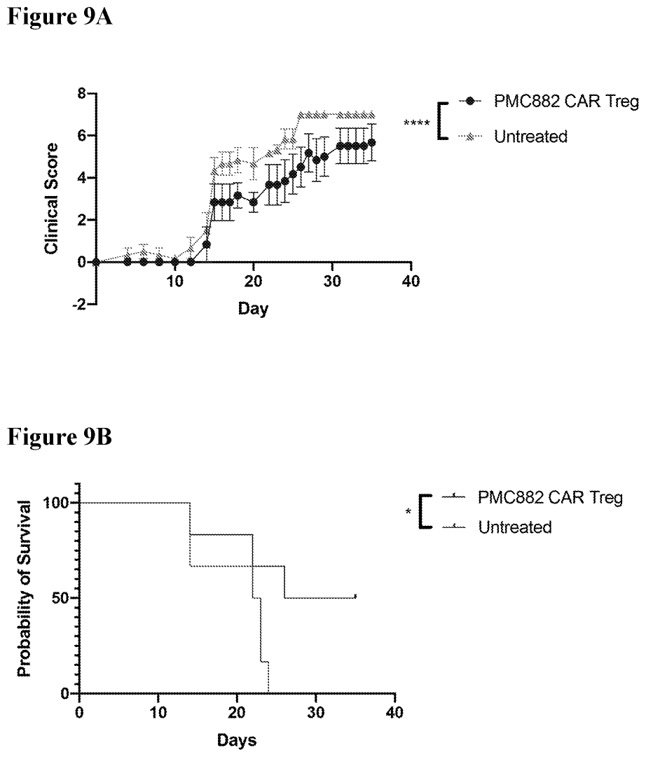

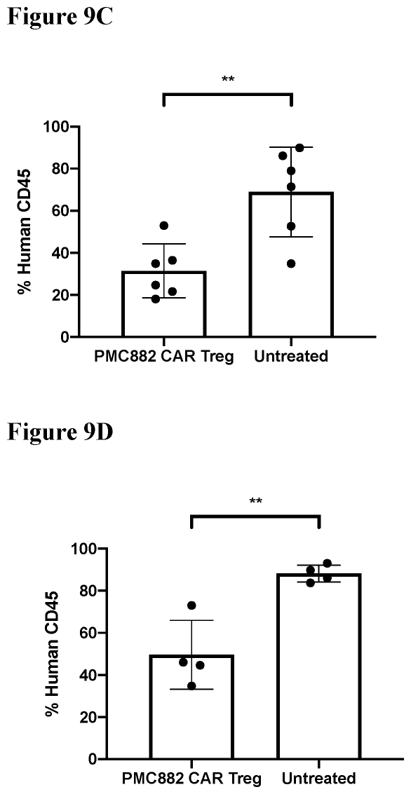

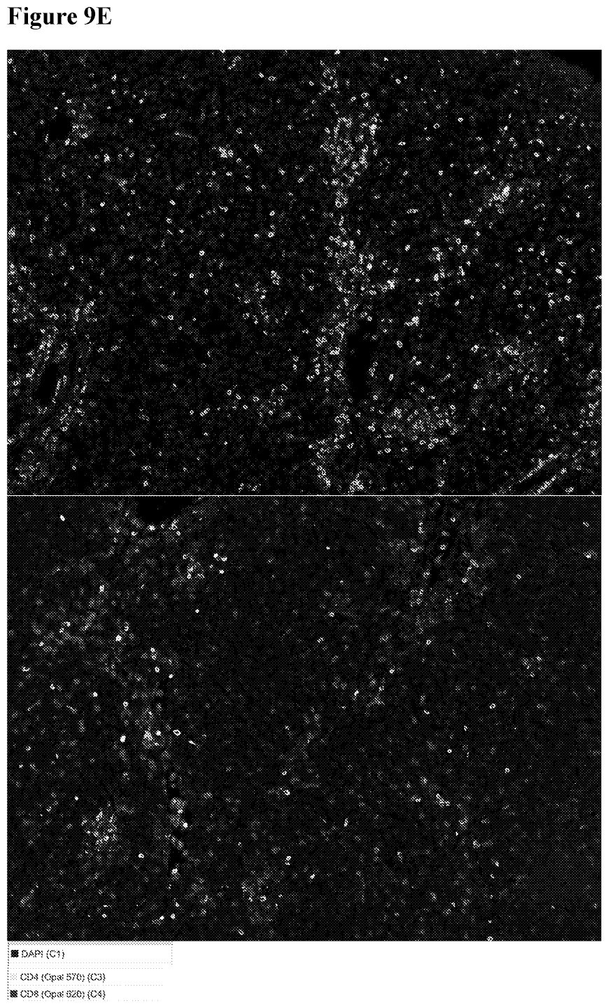

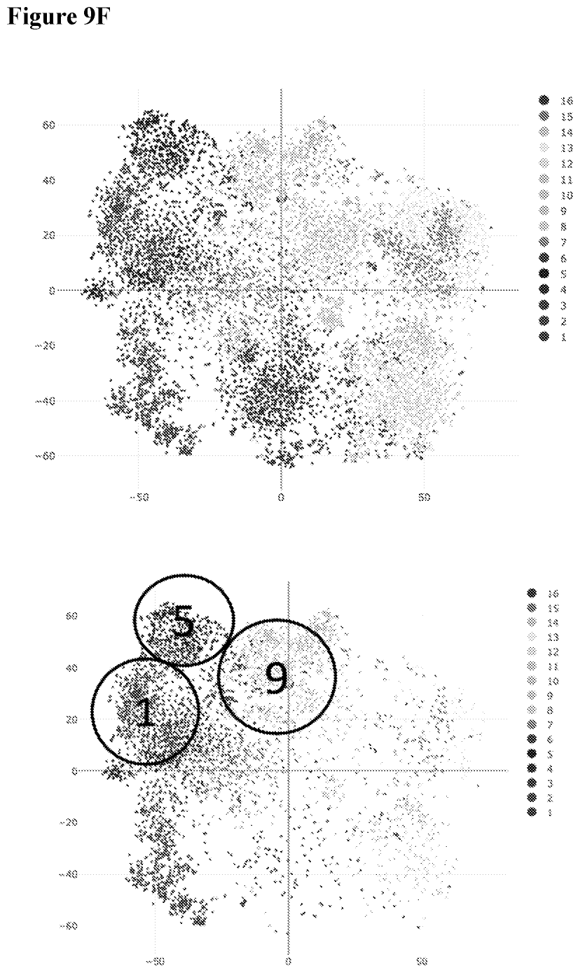

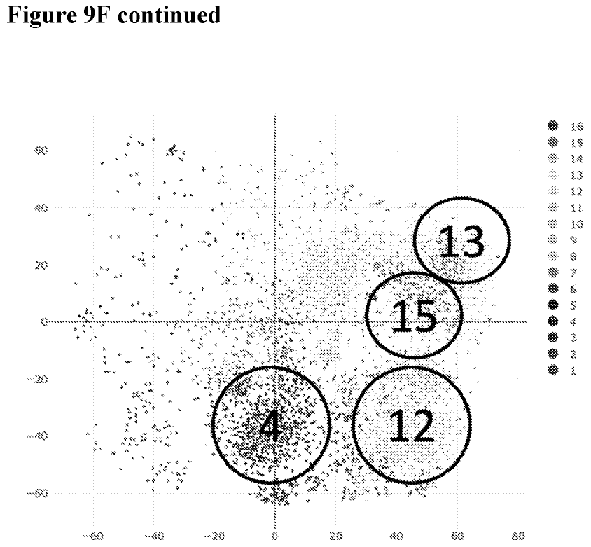

NSG mice were induced with acute GvHD by injecting 5 million human PBMCs at Day 0 and at Day 1, PMC882 CAR Treg treatment was administered. PMC882 CAR Tregs treatment in the NSG GvHD mouse model shows significantly better clinical score (FIG. 9A) and survival (FIG. 9B) compared to untreated mice (in FIG. 9B results show all untreated mice did not survive). Percent of human CD45 engraftment in the mouse peripheral blood, an indicator of the disease progression, shows that PMC882 CAR Treg treated mice are able to significantly suppress human CD45 engraftment at Day 14 (FIG. 9C) and at the end of experiment at Day 35 (FIG. 9D) compared to untreated mice. Liver tissue analysis of CAR Treg treated (top) compared to untreated (bottom) mice shows the difference in human T cell infiltrate into the liver organ where untreated mice show large numbers of predominantly cytotoxic CD8+ T cells (red) whereas CAR Treg treated mice show more CD4+ T cells (yellow) demonstrating a change in the tissue environment brought about by the CAR Treg treatment (FIG. 9E). High dimensionality reduction analysis tSNE plot (top) of mass CyToF analysis of the human CD3 positive cells from mouse peripheral blood shows a clear separation between CAR Treg treated mice (middle) and untreated mice (bottom) (FIG. 9F). Heatmap of the markers found in clusters 1, 5 and 9 show naive T cell markers of CD45RA and CD62L are enriched in the CAR Treg treated mice, indicating T cells that are not previously activated or actively proliferating. Clusters 4, 12, 13 and 15 enriched in the untreated mice had high expression of memory T cell markers CD45RO, activation markers of CD69 and Granzyme B, proliferation markers Ki67 and exhaustion markers LAG3 which indicates T cells that have been activated and stimulated as well as actively proliferating in the untreated mice (FIG. 9G).

### Example 12—Anti-IFNy CAR is Reactive to Soluble Human Interferon Gamma

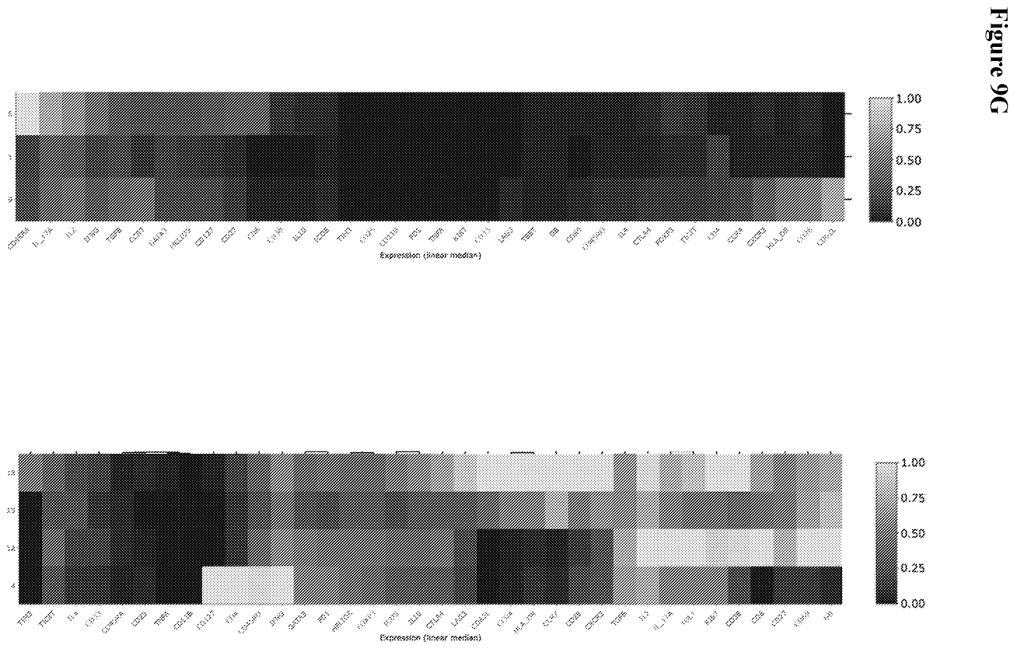

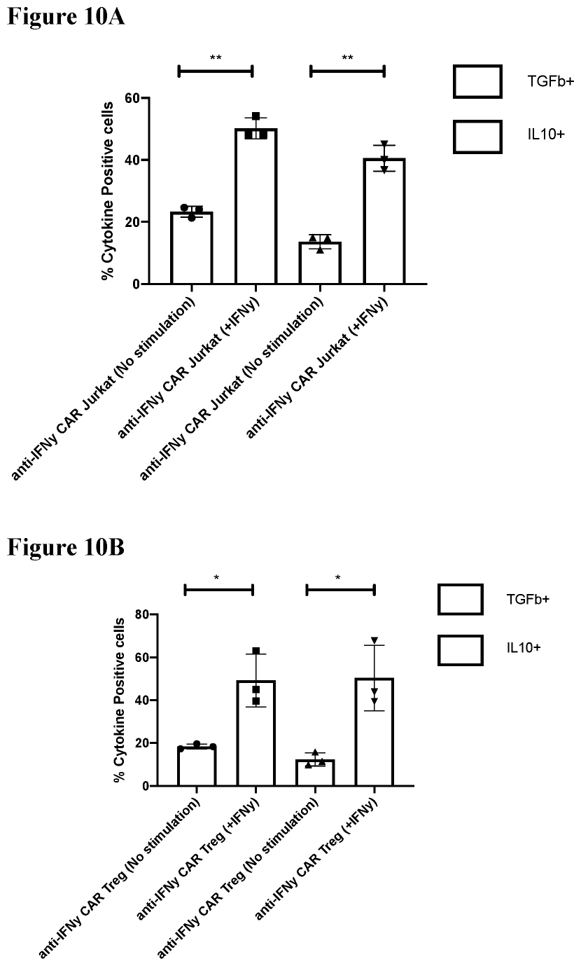

Anti-IFNy CAR construct transduced into CAR Jurkat cells (FIG. 10A), anti-IFNy CAR Tregs from healthy donor (FIG. 10B) showed an increase of TGF beta and IL10 positive cells in the presence of human IFNy at 50 ng/ml.

### Example 13—CAR Tregs are Superior to Expanded Tregs in Suppressing Effector T Cell Proliferation

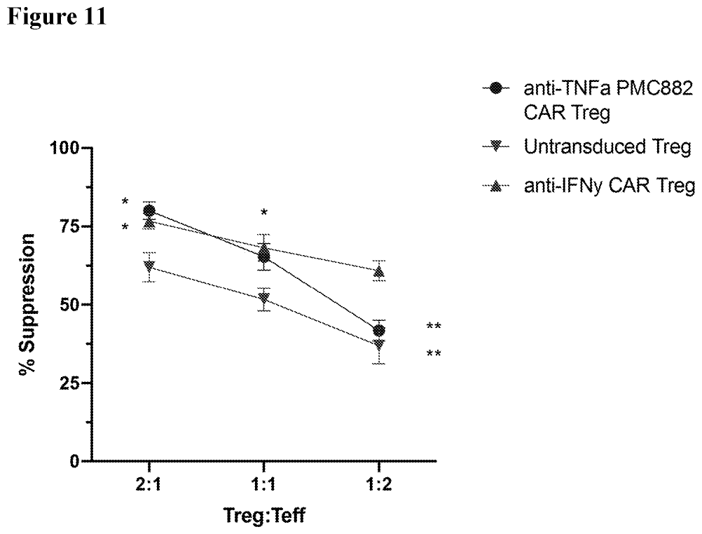

Anti-TNFa CAR PMC882 and anti-IFNy CAR transduced into healthy regulatory T cells demonstrate superior suppression of activated effector T cells compared untransduced Tregs (FIG. 11). For anti-TNFa CAR PMC882 Tregs, significant suppression over untransduced Tregs was seen at 2 Tregs to 1 Teff. For anti-IFNy CAR Tregs, significant suppression over untransduced Tregs was seen at all ratios tested of Tregs to Teff of 2 to 1, 1 to 1 and 1 to 2.

### Example 14—Engineered iPS Cells Demonstrate MHC I and MHC II Knockout and CD47 Expression and are Hypoimmunogenic

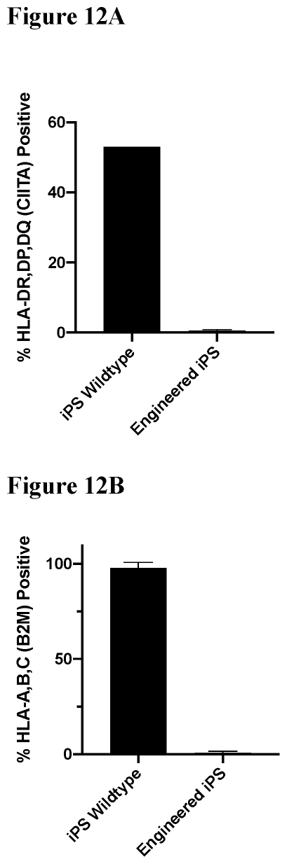

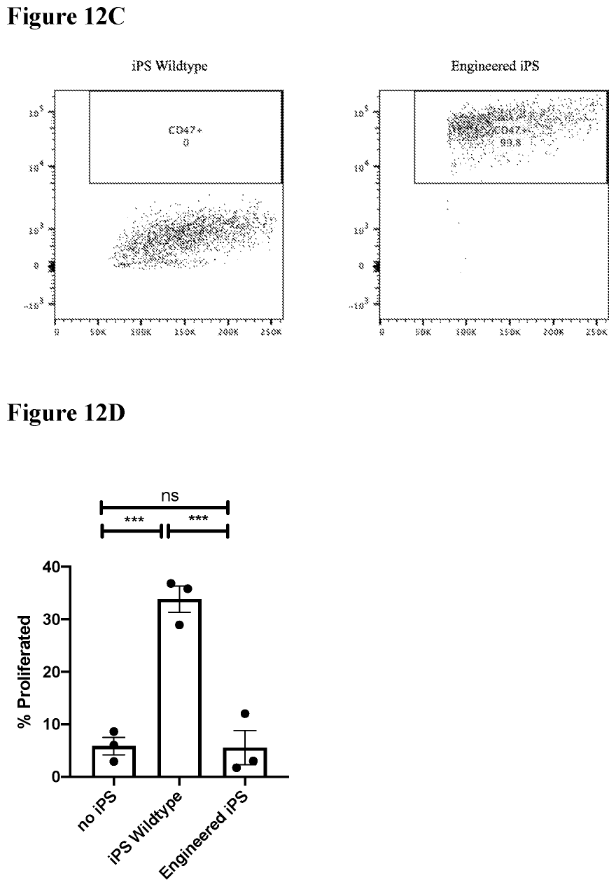

Induced pluripotent stem cells (iPSCs) had major histocompatibility class I (MHC I) gene B2M and class II (MHC II) gene CIITA ablated using CRISPR. Anti-HLA A, B, C (FIG. 12A) and anti-HLA-DR, DP, DQ (FIG. 12B) shows the protein knockdown of their respective genes. CD47 was knocked into the AAVS 1 locus under an overexpression promotor should high expression of CD47 compared to wildtype iPS (FIG. 12C). Genetically edited iPS are shown to be hypoimmunogenic to CD3+ T cells compared to wildtype iPS cells as they do not induce T cell proliferation during co-culture (FIG. 12D).

### Example 15—T Cells Derived from iPS have T Cell Markers and are Inducible Upon T Cell Stimulation

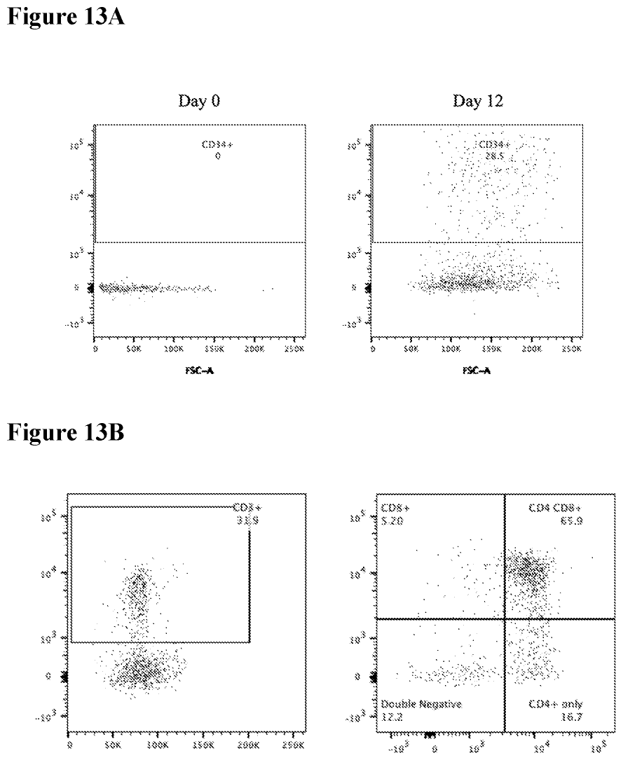

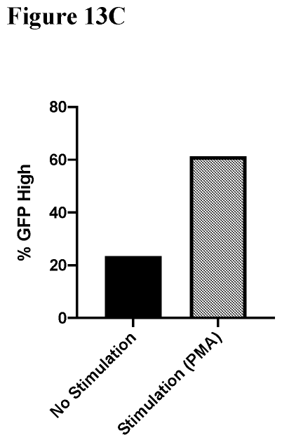

Induced pluripotent cells are differentiated to CD34+ cells (FIG. 13A) then subsequently to T cells, expressing CD3, CD4 and CD8 at Day 24 of differentiation (FIG. 13B). These iPS-derived T cells can also be transduced with lentivirus containing the anti-TNFa CAR with the GFP under the NFAT inducible promotor and upon activation, show an increase in GFP signal demonstrating inducibility of the NFAT promotor in these T cells (FIG. 13C).

1. A chimeric antigen receptor (CAR) comprising an extracellular domain, a transmembrane domain, and an intracellular domain, wherein the extracellular domain binds one or more inflammation associated factor(s).

2. The chimeric antigen receptor of claim 1, wherein the one or more inflammation associated factor(s) is/are selected from the group consisting of Interleukin 1 (IL-1), Tumour Necrosis Factor alpha (TNF-alpha), Interferon gamma (IFN-gamma), Interleukin 12 (IL-12), Interleukin 18 (IL-18), Granulocyte-macrophage colony-stimulating factor (GMCSF), Interleukin 6 (IL-6), Interleukin 8 (IL-8), Interleukin 17A (IL-17), CXCL1, CXCL2, CXCL9, CXCL10, CXCL11, CXCL16, and CCL2-20.

3. The chimeric antigen receptor of claim 1 or 2, wherein the extracellular domain comprises one or more antigen binding domains, wherein the/each antigen binding domain is independently selected from the group consisting of an antibody, an antibody fragment, a single-chain variable fragment (scFv), a chemokine receptor, or functional variations thereof.

4. The chimeric antigen receptor of claim 3, wherein the chemokine receptor is selected from the group consisting of CCR5, CXCR3, CCR1, and CCR2.

5. The chimeric antigen receptor of any one of claims 3 to 4, wherein the/each antigen binding domain is a single-chain variable fragment (scFv).

6. The chimeric antigen receptor of any one of claim 1 to 5, wherein the extracellular domain comprises any one or more of the below:
i) a scFv which binds TNF-alpha,
ii) a scFv which binds IFN-gamma; and
iii) a scFv which binds IL-17.

7. The chimeric antigen receptor of any one of claim 1 to 6, wherein the antigen binding domain competes with an antibody comprising SEQ ID NO: 1; 19 and 20; 64; 65 and 66; 80 and 81; 85 and 86; and 162 and 163 for binding to TNF-alpha.

8. The chimeric antigen receptor of any one of claim 1 to 6, wherein the antigen binding domain competes with an antibody comprising SEQ ID NO: 3; 5; 34 and 35; 49 and 50; 100 and 101; 112 and 113 for binding to IFN-gamma.

9. The chimeric antigen receptor of any one of claim 1 to 6, wherein the antigen binding domain competes with an antibody comprising SEQ ID NO: 127 and 128; 141 and 142 for binding to IL-17a.

10. The chimeric antigen receptor of any one of claim 1 to 6, the antigen recognition domain comprises:
(a) a light chain variable domain (VL) comprising three complementarity determining regions (CDRs) of the amino acid sequence shown in SEQ ID NO: 20, 35, 50, 66, 81, 86, 101, 113, 128, 142 or 163; and
(b) a heavy chain variable domain (VH) comprising three complementarity determining regions (CDRs) of the amino acid sequence shown in SEQ ID NO: 19, 34, 49, 65, 80, 85, 100, 112, 127, 141 or 162.

11. The chimeric antigen receptor of any one of claim 1 to 6, wherein the antigen recognition domain comprises:
(a) a light chain variable domain (VL) comprising three complementarity determining regions (CDRs) of the amino acid sequence shown in SEQ ID NO: 20 or SEQ ID NO: 163; and
(b) a heavy chain variable domain (VH) comprising three complementarity determining regions (CDRs) of the amino acid sequence shown in SEQ ID NO: 19 or SEQ ID NO: 162.

12. The chimeric antigen receptor of any one of claim 1 to 6, wherein the antigen recognition domain comprises:
(a) a light chain variable domain (VL) comprising three complementarity determining regions (CDRs) of the amino acid sequence shown in SEQ ID NO: 35; and
(b) a heavy chain variable domain (VH) comprising three complementarity determining regions (CDRs) of the amino acid sequence shown in SEQ ID NO: 34.

13. The chimeric antigen receptor of any one of claim 1 to 6, wherein the antigen recognition domain comprises:
(a) a light chain variable domain (VL) comprising three complementarity determining regions (CDRs) of the amino acid sequence shown in SEQ ID NO: 50; and
(b) a heavy chain variable domain (VH) comprising three complementarity determining regions (CDRs) of the amino acid sequence shown in SEQ ID NO: 49.

14. The chimeric antigen receptor of any one of claim 1 to 6, wherein the antigen recognition domain comprises:
(a) a light chain variable domain (VL) comprising three complementarity determining regions (CDRs) of the amino acid sequence shown in SEQ ID NO: 66; and
(b) a heavy chain variable domain (VH) comprising three complementarity determining regions (CDRs) of the amino acid sequence shown in SEQ ID NO: 65.

15. The chimeric antigen receptor of any one of claim 1 to 6, wherein the antigen recognition domain comprises:
(a) a light chain variable domain (VL) comprising three complementarity determining regions (CDRs) of the amino acid sequence shown in SEQ ID NO: 81; and
(b) a heavy chain variable domain (VH) comprising three complementarity determining regions (CDRs) of the amino acid sequence shown in SEQ ID NO: 80.

16. The chimeric antigen receptor of any one of claim 1 to 6, wherein the antigen recognition domain comprises:
(a) a light chain variable domain (VL) comprising three complementarity determining regions (CDRs) of the amino acid sequence shown in SEQ ID NO: 86; and
(b) a heavy chain variable domain (VH) comprising three complementarity determining regions (CDRs) of the amino acid sequence shown in SEQ ID NO: 85.

17. The chimeric antigen receptor of any one of claim 1 to 6, wherein the antigen recognition domain comprises:
(a) a light chain variable domain (VL) comprising three complementarity determining regions (CDRs) of the amino acid sequence shown in SEQ ID NO: 101; and
(b) a heavy chain variable domain (VH) comprising three complementarity determining regions (CDRs) of the amino acid sequence shown in SEQ ID NO: 100.

18. The chimeric antigen receptor of any one of claim 1 to 6, wherein the antigen recognition domain comprises:
(a) a light chain variable domain (VL) comprising three complementarity determining regions (CDRs) of the amino acid sequence shown in SEQ ID NO: 113; and
(b) a heavy chain variable domain (VH) comprising three complementarity determining regions (CDRs) of the amino acid sequence shown in SEQ ID NO: 112.

19. The chimeric antigen receptor of any one of claim 1 to 6, wherein the antigen recognition domain comprises:
(a) a light chain variable domain (VL) comprising three complementarity determining regions (CDRs) of the amino acid sequence shown in SEQ ID NO: 128; and
(b) a heavy chain variable domain (VH) comprising three complementarity determining regions (CDRs) of the amino acid sequence shown in SEQ ID NO: 127.

20. The chimeric antigen receptor of any one of claim 1 to 6, wherein the antigen recognition domain comprises:
(a) a light chain variable domain (VL) comprising three complementarity determining regions (CDRs) of the amino acid sequence shown in SEQ ID NO: 142; and
(b) a heavy chain variable domain (VH) comprising three complementarity determining regions (CDRs) of the amino acid sequence shown in SEQ ID NO: 141.

21. The chimeric antigen receptor of any one of claim 1 to 6, wherein the antigen recognition domain comprises:
(a) a light chain variable domain (VL) comprising a complementarity determining region (CDR) 1 comprising or consisting of an amino acid sequence of SEQ ID NOs: 29, 30, 44, 45, 59, 60, 75, 76, 95, 96, 107, 108, 122, 123, 137, 138, 151 or 152; a CDR2 comprising or consisting of an amino acid sequence of SEQ ID NOs: 31, 32, 46, 47, 61, 62, 77, 78, 97, 98, 109, 110, 124, 125, 139 or 153; a CDR3 comprising or consisting of an amino acid sequence of SEQ ID NOs: 33, 48, 63, 79, 84, 99, 111, 126, 140 or 154; and
(b) a heavy chain variable domain (VH) comprising a complementarity determining region (CDR) 1 comprising or consisting of an amino acid sequence of SEQ ID NOs: 21, 22, 23, 36, 37, 38, 51, 52, 53, 67, 68, 69, 87, 88, 89, 114, 115, 116, 129, 130, 131, 143, 144 or 145; a CDR2 comprising or consisting of an amino acid sequence of SEQ ID NOs: 24, 25, 26, 39, 40, 41, 54, 55, 56, 70, 71, 72, 90, 91, 92, 102, 103, 104, 117, 118, 119, 132, 133, 134, 146, 147 or 148; a CDR3 comprising or consisting of an amino acid sequence of SEQ ID NOs: 27, 28, 42, 43, 57, 58, 73, 74, 82, 83, 93, 94, 105, 106, 120, 121, 135, 136, 149 or 150.

22. The chimeric antigen receptor of any one of claim 1 to 6, wherein the antigen recognition domain comprises:
(a) a light chain variable domain (VL) comprising a complementarity determining region (CDR) 1 comprising or consisting of an amino acid sequence of SEQ ID NOs: 29; a CDR2 comprising or consisting of an amino acid sequence of SEQ ID NO: 31; a CDR3 comprising or consisting of an amino acid sequence of SEQ ID NO: 33; and
(b) a heavy chain variable domain (VH) comprising a complementarity determining region (CDR) 1 comprising or consisting of an amino acid sequence of SEQ ID NO: 21; a CDR2 comprising or consisting of an amino acid sequence of SEQ ID NO: 24; a CDR3 comprising or consisting of an amino acid sequence of SEQ ID NO: 27, or
(c) a light chain variable domain (VL) comprising a complementarity determining region (CDR) 1 comprising or consisting of an amino acid sequence of SEQ ID NOs: 29; a CDR2 comprising or consisting of an amino acid sequence of SEQ ID NO: 31; a CDR3 comprising or consisting of an amino acid sequence of SEQ ID NO: 33; and
(d) a heavy chain variable domain (VH) comprising a complementarity determining region (CDR) 1 comprising or consisting of an amino acid sequence of SEQ ID NO: 22; a CDR2 comprising or consisting of an amino acid sequence of SEQ ID NO: 25; a CDR3 comprising or consisting of an amino acid sequence of SEQ ID NO: 27, or
(e) a light chain variable domain (VL) comprising a complementarity determining region (CDR) 1 comprising or consisting of an amino acid sequence of SEQ ID NOs: 30; a CDR2 comprising or consisting of an amino acid sequence of SEQ ID NO: 32; a CDR3 comprising or consisting of an amino acid sequence of SEQ ID NO: 33; and
(f) a heavy chain variable domain (VH) comprising a complementarity determining region (CDR) 1 comprising or consisting of an amino acid sequence of SEQ ID NO: 23; a CDR2 comprising or consisting of an amino acid sequence of SEQ ID NO: 26; a CDR3 comprising or consisting of an amino acid sequence of SEQ ID NO: 28.

23. The chimeric antigen receptor of any one of claim 1 to 6, wherein the antigen recognition domain comprises:
(a) a light chain variable domain (VL) comprising a complementarity determining region (CDR) 1 comprising or consisting of an amino acid sequence of SEQ ID NOs: 44; a CDR2 comprising or consisting of an amino acid sequence of SEQ ID NO: 46; a CDR3 comprising or consisting of an amino acid sequence of SEQ ID NO: 48; and
(b) a heavy chain variable domain (VH) comprising a complementarity determining region (CDR) 1 comprising or consisting of an amino acid sequence of SEQ ID NO: 36; a CDR2 comprising or consisting of an amino acid sequence of SEQ ID NO: 39; a CDR3 comprising or consisting of an amino acid sequence of SEQ ID NO: 42, or
(c) a light chain variable domain (VL) comprising a complementarity determining region (CDR) 1 comprising or consisting of an amino acid sequence of SEQ ID NOs: 44; a CDR2 comprising or consisting of an amino acid sequence of SEQ ID NO: 46; a CDR3 comprising or consisting of an amino acid sequence of SEQ ID NO: 48; and
(d) a heavy chain variable domain (VH) comprising a complementarity determining region (CDR) 1 comprising or consisting of an amino acid sequence of SEQ ID NO: 37; a CDR2 comprising or consisting of an amino acid sequence of SEQ ID NO: 40; a CDR3 comprising or consisting of an amino acid sequence of SEQ ID NO: 42, or
(e) a light chain variable domain (VL) comprising a complementarity determining region (CDR) 1 comprising or consisting of an amino acid sequence of SEQ ID NO: 45; a CDR2 comprising or consisting of an amino acid sequence of SEQ ID NO: 47; a CDR3 comprising or consisting of an amino acid sequence of SEQ ID NO: 48; and
(f) a heavy chain variable domain (VH) comprising a complementarity determining region (CDR) 1 comprising or consisting of an amino acid sequence of SEQ ID NO: 38; a CDR2 comprising or consisting of an amino acid sequence of SEQ ID NO: 41; a CDR3 comprising or consisting of an amino acid sequence of SEQ ID NO: 43.

24. The chimeric antigen receptor of any one of claim 1 to 6, wherein the antigen recognition domain comprises:
(a) a light chain variable domain (VL) comprising a complementarity determining region (CDR) 1 comprising or consisting of an amino acid sequence of SEQ ID NOs: 59; a CDR2 comprising or consisting of an amino acid sequence of SEQ ID NO: 61; a CDR3 comprising or consisting of an amino acid sequence of SEQ ID NO: 63; and
(b) a heavy chain variable domain (VH) comprising a complementarity determining region (CDR) 1 comprising or consisting of an amino acid sequence of SEQ ID NO: 51; a CDR2 comprising or consisting of an amino acid sequence of SEQ ID NO: 54; a CDR3 comprising or consisting of an amino acid sequence of SEQ ID NO: 57, or
(c) a light chain variable domain (VL) comprising a complementarity determining region (CDR) 1 comprising or consisting of an amino acid sequence of SEQ ID NOs: 59; a CDR2 comprising or consisting of an amino acid sequence of SEQ ID NO: 61; a CDR3 comprising or consisting of an amino acid sequence of SEQ ID NO: 63; and
(d) a heavy chain variable domain (VH) comprising a complementarity determining region (CDR) 1 comprising or consisting of an amino acid sequence of SEQ ID NO: 52; a CDR2 comprising or consisting of an amino acid sequence of SEQ ID NO: 55; a CDR3 comprising or consisting of an amino acid sequence of SEQ ID NO: 57, or
(e) a light chain variable domain (VL) comprising a complementarity determining region (CDR) 1 comprising or consisting of an amino acid sequence of SEQ ID NOs: 60; a CDR2 comprising or consisting of an amino acid sequence of SEQ ID NO: 62; a CDR3 comprising or consisting of an amino acid sequence of SEQ ID NO: 63; and
(f) a heavy chain variable domain (VH) comprising a complementarity determining region (CDR) 1 comprising or consisting of an amino acid sequence of SEQ ID NO: 53; a CDR2 comprising or consisting of an amino acid sequence of SEQ ID NO: 56; a CDR3 comprising or consisting of an amino acid sequence of SEQ ID NO: 58.

25. The chimeric antigen receptor of any one of claim 1 to 6, wherein the antigen recognition domain comprises:
(a) a light chain variable domain (VL) comprising a complementarity determining region (CDR) 1 comprising or consisting of an amino acid sequence of SEQ ID NOs: 75; a CDR2 comprising or consisting of an amino acid sequence of SEQ ID NO: 77; a CDR3 comprising or consisting of an amino acid sequence of SEQ ID NO: 79; and
(b) a heavy chain variable domain (VH) comprising a complementarity determining region (CDR) 1 comprising or consisting of an amino acid sequence of SEQ ID NO: 67; a CDR2 comprising or consisting of an amino acid sequence of SEQ ID NO: 70; a CDR3 comprising or consisting of an amino acid sequence of SEQ ID NO: 73, or
(c) a light chain variable domain (VL) comprising a complementarity determining region (CDR) 1 comprising or consisting of an amino acid sequence of SEQ ID NOs: 75; a CDR2 comprising or consisting of an amino acid sequence of SEQ ID NO: 77; a CDR3 comprising or consisting of an amino acid sequence of SEQ ID NO: 79; and
(d) a heavy chain variable domain (VH) comprising a complementarity determining region (CDR) 1 comprising or consisting of an amino acid sequence of SEQ ID NO: 68; a CDR2 comprising or consisting of an amino acid sequence of SEQ ID NO: 71; a CDR3 comprising or consisting of an amino acid sequence of SEQ ID NO: 73, or
(e) a light chain variable domain (VL) comprising a complementarity determining region (CDR) 1 comprising or consisting of an amino acid sequence of SEQ ID NOs: 76; a CDR2 comprising or consisting of an amino acid sequence of SEQ ID NO: 78; a CDR3 comprising or consisting of an amino acid sequence of SEQ ID NO: 79; and
(f) a heavy chain variable domain (VH) comprising a complementarity determining region (CDR) 1 comprising or consisting of an amino acid sequence of SEQ ID NO: 69; a CDR2 comprising or consisting of an amino acid sequence of SEQ ID NO: 72; a CDR3 comprising or consisting of an amino acid sequence of SEQ ID NO: 74.

26. The chimeric antigen receptor of any one of claim 1 to 6, wherein the antigen recognition domain comprises:
(a) a light chain variable domain (VL) comprising a complementarity determining region (CDR) 1 comprising or consisting of an amino acid sequence of SEQ ID NOs: 75; a CDR2 comprising or consisting of an amino acid sequence of SEQ ID NO: 77; a CDR3 comprising or consisting of an amino acid sequence of SEQ ID NO: 84; and
(b) a heavy chain variable domain (VH) comprising a complementarity determining region (CDR) 1 comprising or consisting of an amino acid sequence of SEQ ID NO: 67; a CDR2 comprising or consisting of an amino acid sequence of SEQ ID NO: 70; a CDR3 comprising or consisting of an amino acid sequence of SEQ ID NO: 82, or
(c) a light chain variable domain (VL) comprising a complementarity determining region (CDR) 1 comprising or consisting of an amino acid sequence of SEQ ID NOs: 75; a CDR2 comprising or consisting of an amino acid sequence of SEQ ID NO: 77; a CDR3 comprising or consisting of an amino acid sequence of SEQ ID NO: 84; and
(d) a heavy chain variable domain (VH) comprising a complementarity determining region (CDR) 1 comprising or consisting of an amino acid sequence of SEQ ID NO: 68; a CDR2 comprising or consisting of an amino acid sequence of SEQ ID NO: 71; a CDR3 comprising or consisting of an amino acid sequence of SEQ ID NO: 82, or
(e) a light chain variable domain (VL) comprising a complementarity determining region (CDR) 1 comprising or consisting of an amino acid sequence of SEQ ID NOs: 76; a CDR2 comprising or consisting of an amino acid sequence of SEQ ID NO: 78; a CDR3 comprising or consisting of an amino acid sequence of SEQ ID NO: 84; and
(f) a heavy chain variable domain (VH) comprising a complementarity determining region (CDR) 1 comprising or consisting of an amino acid sequence of SEQ ID NO: 69; a CDR2 comprising or consisting of an amino acid sequence of SEQ ID NO: 72; a CDR3 comprising or consisting of an amino acid sequence of SEQ ID NO: 83.

29. The chimeric antigen receptor of any one of claim 1 to 6, wherein the antigen recognition domain comprises:
(a) a light chain variable domain (VL) comprising a complementarity determining region (CDR) 1 comprising or consisting of an amino acid sequence of SEQ ID NOs: 95; a CDR2 comprising or consisting of an amino acid sequence of SEQ ID NO: 97; a CDR3 comprising or consisting of an amino acid sequence of SEQ ID NO: 99; and
(b) a heavy chain variable domain (VH) comprising a complementarity determining region (CDR) 1 comprising or consisting of an amino acid sequence of SEQ ID NO: 87; a CDR2 comprising or consisting of an amino acid sequence of SEQ ID NO: 90; a CDR3 comprising or consisting of an amino acid sequence of SEQ ID NO: 93, or
(c) a light chain variable domain (VL) comprising a complementarity determining region (CDR) 1 comprising or consisting of an amino acid sequence of SEQ ID NOs: 95; a CDR2 comprising or consisting of an amino acid sequence of SEQ ID NO: 97; a CDR3 comprising or consisting of an amino acid sequence of SEQ ID NO: 99; and
(d) a heavy chain variable domain (VH) comprising a complementarity determining region (CDR) 1 comprising or consisting of an amino acid sequence of SEQ ID NO: 88; a CDR2 comprising or consisting of an amino acid sequence of SEQ ID NO: 91; a CDR3 comprising or consisting of an amino acid sequence of SEQ ID NO: 93, or
(e) a light chain variable domain (VL) comprising a complementarity determining region (CDR) 1 comprising or consisting of an amino acid sequence of SEQ ID NOs: 96; a CDR2 comprising or consisting of an amino acid sequence of SEQ ID NO: 98; a CDR3 comprising or consisting of an amino acid sequence of SEQ ID NO: 99; and
(f) a heavy chain variable domain (VH) comprising a complementarity determining region (CDR) 1 comprising or consisting of an amino acid sequence of SEQ ID NO: 89; a CDR2 comprising or consisting of an amino acid sequence of SEQ ID NO: 92; a CDR3 comprising or consisting of an amino acid sequence of SEQ ID NO: 94.

30. The chimeric antigen receptor of any one of claim 1 to 6, wherein the antigen recognition domain comprises:
(a) a light chain variable domain (VL) comprising a complementarity determining region (CDR) 1 comprising or consisting of an amino acid sequence of SEQ ID NOs: 107; a CDR2 comprising or consisting of an amino acid sequence of SEQ ID NO: 109; a CDR3 comprising or consisting of an amino acid sequence of SEQ ID NO: 11; and
(b) a heavy chain variable domain (VH) comprising a complementarity determining region (CDR) 1 comprising or consisting of an amino acid sequence of SEQ ID NO: 21; a CDR2 comprising or consisting of an amino acid sequence of SEQ ID NO: 102; a CDR3 comprising or consisting of an amino acid sequence of SEQ ID NO: 105, or
(c) a light chain variable domain (VL) comprising a complementarity determining region (CDR) 1 comprising or consisting of an amino acid sequence of SEQ ID NOs: 107; a CDR2 comprising or consisting of an amino acid sequence of SEQ ID NO: 109; a CDR3 comprising or consisting of an amino acid sequence of SEQ ID NO: 111; and
(d) a heavy chain variable domain (VH) comprising a complementarity determining region (CDR) 1 comprising or consisting of an amino acid sequence of SEQ ID NO: 22; a CDR2 comprising or consisting of an amino acid sequence of SEQ ID NO: 103; a CDR3 comprising or consisting of an amino acid sequence of SEQ ID NO: 105, or
(e) a light chain variable domain (VL) comprising a complementarity determining region (CDR) 1 comprising or consisting of an amino acid sequence of SEQ ID NOs: 108; a CDR2 comprising or consisting of an amino acid sequence of SEQ ID NO: 110; a CDR3 comprising or consisting of an amino acid sequence of SEQ ID NO: 111; and
(f) a heavy chain variable domain (VH) comprising a complementarity determining region (CDR) 1 comprising or consisting of an amino acid sequence of SEQ ID NO: 23; a CDR2 comprising or consisting of an amino acid sequence of SEQ ID NO: 104; a CDR3 comprising or consisting of an amino acid sequence of SEQ ID NO: 106.

31. The chimeric antigen receptor of any one of claim 1 to 6, wherein the antigen recognition domain comprises:
(a) a light chain variable domain (VL) comprising a complementarity determining region (CDR) 1 comprising or consisting of an amino acid sequence of SEQ ID NOs: 122; a CDR2 comprising or consisting of an amino acid sequence of SEQ ID NO: 124; a CDR3 comprising or consisting of an amino acid sequence of SEQ ID NO: 126; and
(b) a heavy chain variable domain (VH) comprising a complementarity determining region (CDR) 1 comprising or consisting of an amino acid sequence of SEQ ID NO: 114; a CDR2 comprising or consisting of an amino acid sequence of SEQ ID NO: 117; a CDR3 comprising or consisting of an amino acid sequence of SEQ ID NO: 120, or
(c) a light chain variable domain (VL) comprising a complementarity determining region (CDR) 1 comprising or consisting of an amino acid sequence of SEQ ID NOs: 122; a CDR2 comprising or consisting of an amino acid sequence of SEQ ID NO: 124; a CDR3 comprising or consisting of an amino acid sequence of SEQ ID NO: 126; and
(d) a heavy chain variable domain (VH) comprising a complementarity determining region (CDR) 1 comprising or consisting of an amino acid sequence of SEQ ID NO: 1; a CDR2 comprising or consisting of an amino acid sequence of SEQ ID NO: 18; a CDR3 comprising or consisting of an amino acid sequence of SEQ ID NO: 120, or
(e) a light chain variable domain (VL) comprising a complementarity determining region (CDR) 1 comprising or consisting of an amino acid sequence of SEQ ID NOs: 123; a CDR2 comprising or consisting of an amino acid sequence of SEQ ID NO: 125; a CDR3 comprising or consisting of an amino acid sequence of SEQ ID NO: 126; and
(f) a heavy chain variable domain (VH) comprising a complementarity determining region (CDR) 1 comprising or consisting of an amino acid sequence of SEQ ID NO: 116; a CDR2 comprising or consisting of an amino acid sequence of SEQ ID NO: 119; a CDR3 comprising or consisting of an amino acid sequence of SEQ ID NO: 121.

32. The chimeric antigen receptor of any one of claim 1 to 6, wherein the antigen recognition domain comprises:
(a) a light chain variable domain (VL) comprising a complementarity determining region (CDR) 1 comprising or consisting of an amino acid sequence of SEQ ID NOs: 137; a CDR2 comprising or consisting of an amino acid sequence of SEQ ID NO: 139; a CDR3 comprising or consisting of an amino acid sequence of SEQ ID NO: 140; and
(b) a heavy chain variable domain (VH) comprising a complementarity determining region (CDR) 1 comprising or consisting of an amino acid sequence of SEQ ID NO: 129; a CDR2 comprising or consisting of an amino acid sequence of SEQ ID NO: 132; a CDR3 comprising or consisting of an amino acid sequence of SEQ ID NO: 135, or
(c) a light chain variable domain (VL) comprising a complementarity determining region (CDR) 1 comprising or consisting of an amino acid sequence of SEQ ID NOs: 137; a CDR2 comprising or consisting of an amino acid sequence of SEQ ID NO: 139; a CDR3 comprising or consisting of an amino acid sequence of SEQ ID NO: 140; and
(d) a heavy chain variable domain (VH) comprising a complementarity determining region (CDR) 1 comprising or consisting of an amino acid sequence of SEQ ID NO: 130; a CDR2 comprising or consisting of an amino acid sequence of SEQ ID NO: 133; a CDR3 comprising or consisting of an amino acid sequence of SEQ ID NO: 135, or
(e) a light chain variable domain (VL) comprising a complementarity determining region (CDR) 1 comprising or consisting of an amino acid sequence of SEQ ID NOs: 138; a CDR2 comprising or consisting of an amino acid sequence of SEQ ID NO: 125; a CDR3 comprising or consisting of an amino acid sequence of SEQ ID NO: 140; and
(f) a heavy chain variable domain (VH) comprising a complementarity determining region (CDR) 1 comprising or consisting of an amino acid sequence of SEQ ID NO: 131; a CDR2 comprising or consisting of an amino acid sequence of SEQ ID NO: 134; a CDR3 comprising or consisting of an amino acid sequence of SEQ ID NO: 136.

33. The chimeric antigen receptor of any one of claim 1 to 6, wherein the antigen recognition domain comprises:
(a) a light chain variable domain (VL) comprising a complementarity determining region (CDR) 1 comprising or consisting of an amino acid sequence of SEQ ID NOs: 151; a CDR2 comprising or consisting of an amino acid sequence of SEQ ID NO: 153; a CDR3 comprising or consisting of an amino acid sequence of SEQ ID NO: 154; and
(b) a heavy chain variable domain (VH) comprising a complementarity determining region (CDR) 1 comprising or consisting of an amino acid sequence of SEQ ID NO: 143; a CDR2 comprising or consisting of an amino acid sequence of SEQ ID NO: 146; a CDR3 comprising or consisting of an amino acid sequence of SEQ ID NO: 149, or
(c) a light chain variable domain (VL) comprising a complementarity determining region (CDR) 1 comprising or consisting of an amino acid sequence of SEQ ID NOs: 151; a CDR2 comprising or consisting of an amino acid sequence of SEQ ID NO: 153; a CDR3 comprising or consisting of an amino acid sequence of SEQ ID NO: 154; and
(d) a heavy chain variable domain (VH) comprising a complementarity determining region (CDR) 1 comprising or consisting of an amino acid sequence of SEQ ID NO: 144; a CDR2 comprising or consisting of an amino acid sequence of SEQ ID NO: 147; a CDR3 comprising or consisting of an amino acid sequence of SEQ ID NO: 149, or
(e) a light chain variable domain (VL) comprising a complementarity determining region (CDR) 1 comprising or consisting of an amino acid sequence of SEQ ID NOs: 152; a CDR2 comprising or consisting of an amino acid sequence of SEQ ID NO: 47; a CDR3 comprising or consisting of an amino acid sequence of SEQ ID NO: 154; and
(f) a heavy chain variable domain (VH) comprising a complementarity determining region (CDR) 1 comprising or consisting of an amino acid sequence of SEQ ID NO: 145; a CDR2 comprising or consisting of an amino acid sequence of SEQ ID NO: 148; a CDR3 comprising or consisting of an amino acid sequence of SEQ ID NO: 150.

34. The chimeric antigen receptor of any one of claim 1 to 6, wherein the antigen binding domain comprises or consists of an amino acid sequence having at least 75%, 80%, 85%, 90%, 91%, 92%, 93%, 94%, 95%, 96%, 97%, 98%, 99%, or any percentage in between of identity to the amino acid sequence as set forth in SEQ ID NO: 1.

35. The chimeric antigen receptor of any one of claim 1 to 6, wherein the antigen binding domain comprises or consists of an amino acid sequence having at least 75%, 80%, 85%, 90%, 91%, 92%, 93%, 94%, 95%, 96%, 97%, 98%, 99%, or any percentage in between of identity to the amino acid sequence as set forth in SEQ ID NO: 3.

36. The chimeric antigen receptor of any one of claim 1 to 6, wherein the antigen binding domain comprises or consists of an amino acid sequence having at least 75%, 80%, 85%, 90%, 91%, 92%, 93%, 94%, 95%, 96%, 97%, 98%, 99%, or any percentage in between of identity to the amino acid sequence as set forth in SEQ ID NO: 5.

37. The chimeric antigen receptor of any one of claim 1 to 6, wherein the antigen binding domain comprises or consists of the amino acid sequence as set forth in SEQ ID: 1.

38. The chimeric antigen receptor of any one of claim 1 to 6, wherein the antigen binding domain comprises or consists of amino acid sequence as set forth in SEQ ID NO: 3.

39. The chimeric antigen receptor of any one of claim 1 to 6, wherein the antigen binding domain comprises or consists of the amino acid sequence as set forth in SEQ ID NO: 5.

40. The chimeric antigen receptor of any one of the preceding claims, wherein the chimeric antigen receptor further comprises a hinge region located between the extracellular domain and the transmembrane domain.

41. The chimeric antigen receptor of claim 40, wherein the hinge region is selected from the group consisting of a CD8a hinge, a CD28 hinge, and an IgG hinge.

42. The chimeric antigen receptor of claim 41, wherein the hinge region is or comprises a CD8a hinge region or an IgG4 hinge.

43. The chimeric antigen receptor of any one of claim 40-41, wherein the hinge region comprises the amino acid sequence of SEQ ID: 7.

44. The chimeric antigen receptor of any one of claims 1-43, wherein the intracellular domain comprises one or more of the following: a signalling domain, one or more co-stimulatory domains, and forkhead box P3 (FOXP3) transcription factor.

45. The chimeric antigen receptor of claim 44, wherein the signalling domain comprises one or more immunoreceptor tyrosine-based activation motifs (ITAMs).

46. The chimeric antigen receptor of claim 44 or 45, wherein the signalling domain comprises an intracellular signalling domain of any one of the proteins selected from the group consisting of TCR zeta, FcR gamma, FcR beta, CD3 gamma, CD3 delta, CD3 epsilon, CD3 zeta, CD22, CD79a, CD79b, CD66d, and functional variations/fragments thereof.

47. The chimeric antigen receptor of claim 44-46, wherein the signalling domain is the intracellular signalling domain of CD3 zeta or a functional variant thereof.

48. The chimeric antigen receptor of any one of claims 44-47, wherein the signalling domain is encoded by the nucleotide sequence set forth in SEQ ID: 5 or a codon redundant sequence thereof.

49. The chimeric antigen receptor of any one of claims 44-48, wherein each of the one or more co-stimulatory domains comprise an intracellular signalling domain of any one of the proteins (for example, co-stimulatory receptor, integrins, NK receptors and the like) selected from the group consisting of: CD28, CD28T, OX40, 4-1BB/CD137, CD2, CD4, CD5, CD7, CD9, CD16, CD22, CD27, CD30, CD33, CD37, CD40, CD45, CD64, CD80, CD86, CD134, CD137, CD154, PD-1, ICOS, lymphocyte function-associated antigen-1 (LFA-1 (CD1 la/CD18), CD247, CD276 (B7-H3), LIGHT (tumour necrosis factor superfamily member 14; TNFSF14), NKG2C, Ig alpha (CD79a), DAP-10, Fc gamma receptor, MHC class I molecule, TNF, TNFr, integrin, signalling lymphocytic activation molecule, BTLA, Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1ld, ITGAE, CD103, IT GAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, CD83 ligand, and functional variations/fragments thereof.

50. The chimeric antigen receptor of claim 49, wherein each of the one or more co-stimulatory domains comprise an intracellular signalling domain of any one of the proteins selected from the group consisting of CD28, 41BB, IL2Rb, TLR2, MyD88, and CD40.

51. The chimeric antigen receptor of claim 50, wherein the one or more co-stimulatory domains comprises the intracellular signalling domain of CD28.

52. The chimeric antigen receptor of any one of claims 1-51, wherein the intracellular domain comprises a co-stimulatory domain, wherein the co-stimulatory domain is or comprises a CD28 intracellular signalling domain comprising the amino acid sequence of SEQ ID NO: 9.

53. The chimeric antigen receptor of any one of the preceding claims, wherein the transmembrane domain is a transmembrane domain of a protein selected from the group consisting of CD28, CD28T, OX-40, 4-1BB/CD137, CD2, CD7, CD27, CD30, CD40, programmed death-1 (PD-1), inducible T cell co-stimulator (ICOS), lymphocyte function-associated antigen-1 (LFA-1, CD11a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, and functional variations/fragments thereof.

54. The chimeric antigen receptor of claim 53, wherein the transmembrane domain is a CD3 epsilon transmembrane domain or a functional variant thereof.

55. The chimeric antigen receptor of any one of claims 1 to 53, wherein the transmembrane domain is a CD28 transmembrane domain comprising or consisting of an amino acid sequence of SEQ ID: 10.

56. The chimeric antigen receptor of any one of the preceding claims, wherein the extracellular domain and/or the intracellular domain comprise a self-cleaving peptide.

57. The chimeric antigen receptor of claim 56, wherein the self-cleaving peptide is selected from the group consisting of P2A, E2A, F2A, and T2A.

58. The chimeric antigen receptor of any one of the preceding claims, wherein the hinge region comprises an IgG4 hinge, the transmembrane domain comprises a CD28 transmembrane domain, the co-stimulatory domain is the intracellular signalling domain of CD28, the signalling domain comprises the intracellular signalling domain of CD3 zeta, the intracellular domain comprises FOXP3, wherein the intracellular signalling domain of CD3 zeta and the FOX3P are separated by the self-cleaving peptide TA2.

59. The chimeric antigen receptor of any one of the preceding claims, wherein the hinge region comprises an IgG4 hinge, the transmembrane domain comprises a CD28 transmembrane domain, the co-stimulatory domain is the intracellular signalling domain of CD28, the signalling domain comprises the intracellular signalling domain of CD3 zeta.

60. The chimeric antigen receptor of any one of the preceding claims, wherein the chimeric antigen receptor comprises the sequence of SEQ ID NO: 12.

61. A first polynucleotide encoding the chimeric antigen receptor of any one of the preceding claims.

62. A vector comprising the first polynucleotide of claim 61.

63. The vector of claim 62, wherein the vector is a viral vector, optionally a lentiviral vector.

64. A modified cell comprising the chimeric antigen receptor of any one of claims 1-60, the first polynucleotide of claim 61, and/or the vector of any one of claims 62-63.

65. The modified cell of claim 64, wherein the modified cell is a modified T cell.

66. The modified cell of claim 65, wherein the modified T cell expresses one or more immunosuppressive molecules.

67. The modified cell of claim 66, wherein the modified T cell expresses one or more immunosuppressive molecules when the modified T cell is activated.

68. The modified cell of claim 67, wherein for the expression of the one or more immunosuppressive molecules the modified T cell comprises a second polynucleotide encoding:
a. a promoter comprising one or more binding sites for NFAT (nuclear factor of activated T cells), and
b. one or more immunosuppressive molecules;
wherein the binding of NFAT to the promoter induces the expression of the one or more immune-suppressive molecules.

69. The modified cell of claim 68, wherein the promoter comprises, or is, the promoter of Interleukin-2 (IL-2), or a derivative thereof.

70. The modified cell of claim 68 or 69, wherein when the second polynucleotide encodes more than one immunosuppressive molecules, the coding sequences of the immunosuppressive molecules are separated by one or more linker sequences.

71. The modified cell of claim 70, wherein the one or more linker sequences encode glycine-serine (GS) linkers, for example GSG linkers.

72. The modified cell of claim 70 or 71, wherein each of the one or more linker sequences further encodes the self-cleaving peptide as defined in any one of claims 24 to 25.

73. The modified cell of any one of claims 65-72, wherein the one or more immunosuppressive molecules are capable of suppressing the activity of effecter T cells or are capable of inducing anergy in effector T cells.

74. The modified cell of any one of claims 65-73, wherein the one or more immunosuppressive molecules are selected from the group consisting of IL-10, TGF-beta, CTLA-4, LAG3, PD-L1, FOXP3, and PD-1.

75. The modified cell of any one of claims 65-74, wherein the one or more immunosuppressive molecules is selected from the group consisting of IL-10, TGF-beta, and FOXP3.

76. The modified cell of claim 75, wherein immunosuppressive molecules are IL-10 and TGF-beta, or IL-10, TGF-beta and FOXP3.

77. The modified cell of any one of claims 68-76, wherein the second polynucleotide comprises the sequence as set forth in SEQ ID: 13.

78. The modified cell of any one of claims 65-77, wherein the modified T cell further comprises a third polynucleotide, said third polynucleotide encoding an inducible suicide system, wherein contacting of an inducer molecule by the modified T cell will induce cell death in said modified T cell.

79. The modified cell of claim 78, wherein the inducible suicide system is a caspase-9 (iCasp9) suicide gene system.

80. The modified cell of claim 78 or 79, wherein the inducer is CID (also known as AP1903/Rimiducid).

81. The modified cell of any one of claims 65-80, wherein the first and second polynucleotides are encoded on one vector or are encoded on different vectors.

82. The modified cell of any one of claims 78-81, wherein the first, second and third polynucleotides are comprised on one vector, or are encoded on two or more different vectors.

83. The modified cell of any one of claims 65-82, wherein the modified T cell is modified from a regulatory T cell (Treg).

84. The modified cell of claim 83, wherein the Treg is isolated from a subject, or wherein the Treg is isolated from a subject and expanded ex vivo.

85. The modified cell of claim 84, wherein when Treg is isolated from a subject, the Treg is isolated from the PBMCs of the subject.

86. The modified cell of any one of claims 65-82, wherein the modified T cell is derived from an induced pluripotent stem cell (iPSC).

87. The modified cell of claim 86, wherein the iPSC is derived from a donor CD34+ iPSC.

88. The modified cell of any one of claims 65-87, wherein the modified T cell is hypoimmunogenic.

89. The modified cell of claim 88, wherein one or more genes of the Major Histocompatibility Class (MHC) I, and one or more genes of the Major Histocompatibility Class (MHC) II are deleted or mutated, wherein the perturbations of the genes result in the dysfunction of the MHC I and MHC II protein complexes.

90. The modified cell of claim 89, wherein the one or more genes of MHC I and II comprise at least B2M (β2 microglobulin) and CIITA (class II MHC transactivator).

91. The modified cell of any one of claims 65-90, wherein the modified T cell is further modified to overexpress CD47 or a functional variant thereof.

92. A method of generating Chimeric antigen receptor (CAR)-regulatory T cells (CAR-Tregs), said method comprising:
c. isolating regulatory T cells (Treg) from peripheral blood mononuclear cells (PBMCs) from a subject;
d. expanding the isolated Tregs ex vivo; and
e. transducing the expanded Tregs with the vector according to claim 29 or 30, so that the transduced Tregs express the CAR of any one of claims 1-27.

93. A method of generating modified hypoimmunogenic iPSC-derived T cells, said method comprising:
f. editing the genome of CD34+-iPSCs to knock-out both B2M (β2 microglobulin) and CIITA (class II MHC transactivator) genes;
g. genetically editing and knocking in CD47 or a functional variant thereof, wherein the knocked in T-iPSC overexpresses CD47;
h. transducing hypoimmunogenic CD34+ iPSCs with the vector of claim 29 or 30, so that the transduced T cells express the chimeric antigen receptor of any one of claims 1-27;
i. differentiating the hypoimmunogenic CAR-iPSCs to generate modified iPSC-derived T cells.

94. The method of claim 93, wherein steps a. and b. are carried out using the CRISPR-Cas gene editing system.

95. The method of any one of claims 92-94, wherein the method further comprises any one or more of the following:
e. testing for the expression of the chimeric antigen receptor of any one of claims 1-27;
f. testing the functional potential of the chimeric antigen receptor-Tregs or CAR-iPSC-derived T cells to produce one or more immunosuppressive molecules in response to contacting the inflammation associated factors; and
g. testing the functional potential of the CAR-Tregs or CAR-iPSC-derived T cells to suppress activation/proliferation of effector T cells.

96. The method of any one of claims 92-95, wherein the vector is a viral vector, preferably a lentiviral vector.

97. The modified cell of any one of claims 65-91 for use in therapy, preferably where the therapy is for treating a disease or condition involving or resulting from uncontrolled inflammatory responses mediated by one or more inflammation associated factor(s).

98. The modified cell of any one of claims 65-91 for use in the treatment of an autoimmune disease; a transplant rejection, a graft versus host disease (GVHD), or a cytokine release syndrome.

99. A method of treating an autoimmune disease, a transplant rejection, a graft versus host disease (GVHD) or a cytokine release syndrome, wherein the method comprises administering a therapeutically effective number of the modified T cell of any one of claims 65-91.

100. The modified cell according to claim 98, or the method according to 99, wherein the autoimmune disease is selected from the group consisting of rheumatoid arthritis, psoriatic arthritis, psoriasis, lupus, juvenile rheumatoid arthritis, multiple sclerosis, inflammatory bowel disease, and Crohn's disease.

101. The modified cell according to claim 98, or the method according to 101, wherein the transplant rejection is selected from the group consisting of organ transplant rejection, stem cell transplant rejection, and bone marrow transplant rejection.

102. A method of inducing immune tolerance in a subject in need thereof, wherein the method comprises administering a therapeutically effective number of the modified cell of any one of claims 65-91.

103. A method of downregulating inflammation locally or systemically in a subject in need thereof, wherein the method comprises administering a therapeutically effective number of the modified cell of any one of claims 65-91.

104. A method of suppressing the activity of effector T cells locally or systematically in a subject, wherein the method comprises administering a modified cell of any one of claims 65-91 to the subject locally or systematically.

105. A vector comprising the sequence as set forth in SEQ ID NO: 14 or 15.


SEQUENCE LISTING (166 sequences)

SEQ ID NO: 1 - protein, 238 aa, Artificial Sequence
  Exemplary amino acid sequence of a TNF-alpha scFv clone P3H2
EVQLLESGGGLVQPGGSLRLSCAASGFTFSSYAMSWVRQAPGKGLEWVSSISSTGASTTYADSVKGRFTISRDNSKNTLYLQMNSLRAEDTAVYYCAKGGAAFDYWGQGTLVTVSSGGGGSGGGGSGGGGSDIQMTQSPSSLSASVGDRVTITCRASQSISSYLNWYQQKPGKAPKLLIYSASYLQSGVPSRFSGSGSGTDFTLTISSLQPEDFATYYCQQANNAPTTFGQGTKVEIK

SEQ ID NO: 2 - DNA, 714 bp, Artificial Sequence
  An exemplary coding sequence for the TNF-alpha scFv clone P3H2
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaagtatttcttctactggtgcttctacaacttacgcagactccgtgaagggcaggttcaccatctccagagacaattccaagaacacgctgtatctgcaaatgaacagcctgagagccgaggacacggccgtatattactgtgcgaaaggtggtgctgcttttgactactggggccagggaaccctggtcaccgtcagcagcggtggaggtggttctggtggaggaggttctggtggcggtggttctgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagtgcatcctatttgcaaagtggggtcccatcaaggttcagtggcagtggatctgggacagatttcactctcaccatcagcagtctgcaacctgaagattttgcaacttactactgtcaacaggctaataatgctcctactacgttcggccaagggaccaaggtggaaatcaaa

SEQ ID NO: 3 - protein, 246 aa, Artificial Sequence
  Exemplary amino acid sequence of an IFN-gamma scFv TII 6AE52
EVKLEQSGTVLARPGASVKMSCKASGYTFTNYWMHWIRQRPGQGLEWIGAIYPGISDTSYNQKFKVKAKLTAVTSTSTAYMELSSLTNEDSAVYYCTRDEDFDGFAYWGQGTLVTVSAGGGGSGGGGSGGGGSDVLMTQTPLSLPVSLGDQASISCRSSQSIVHSNGNTYLDWYLQKPGQSPKLLIYKVSNRFSGVPDRFSGSGSGTDFTLRISRVEAEDLGVYYCFRGSHAPYTFGGGTKLEIKR

SEQ ID NO: 4 - DNA, 738 bp, Artificial Sequence
  An exemplary coding sequence for the IFN-gamma scFv TII 6AE52
gaggtcaagctggagcagtctgggactgtgctggcaaggcctggggcttcagtgaagatgtcctgcaaggcttctggctacacctttaccaactactggatgcattggataagacagaggcctggacagggtctggaatggattggcgctatttatcctggaattagtgatactagttacaaccagaagttcaaggtcaaggccaaactgactgcagtcacatccaccagcactgcctacatggagctcagcagcctgacaaatgaggactctgcggtctattattgtacaagagatgaggatttcgacggctttgcttactggggccaagggactctggtcactgtctctgcaggtggtggtgggtctggcggaggtggtagtggaggtggtgggtctgatgttttgatgacccaaactccactctccctgcctgtcagtcttggagatcaagcctccatctcttgcagatctagtcagagtattgtacatagtaatggaaacacctatttagactggtacctgcagaaaccaggccagtctccaaagctcctgatctacaaagtttccaaccgattttctggggtcccagacaggttcagtggcagtggatcagggacagatttcacactcaggatcagcagagtggaggctgaggatctgggagtttattactgctttcgaggatcacatgctccgtacacgttcggaggggggaccaagctggaaataaaacgg

SEQ ID NO: 5 - protein, 243 aa, Artificial Sequence
  Amino acid sequence of IFN-gamma scFv TII 2G1G7
QVKLEQSGPGLVAPSQSLSITCTVSGFSLTSYGVHWVRQSPGKGLEWLGVIWAGGSTNYNSALMSRLSISKDNSKSQVFLKMNSLQTDDTAMYYCARRDGNRYYTMDYWGQGASVTVSSGGGGSGGGGSGGGGSENVLTQSPAIMSASPGEKVTMTCRASSSVSSSYLHWYQQKSGGSPKLWIYRTSNLASGVPARFSGSGSGTSYSLTISSVEAEDAATYYCQQYSGYPLTFGAGTKLELKR

SEQ ID NO: 6 - DNA, 729 bp, Artificial Sequence
  An exemplary coding sequence for the IFN-gamma scFv TII 2G1G7
caagttaagctggagcagtcaggacctggcctggtggcgccctcacagagcctgtccatcacttgcactgtctctgggttttcattaaccagctatggtgtacactgggttcgccagtctccaggaaagggtctggagtggctgggagtaatatgggctggtggaagcacaaattataattcggctctcatgtccagactgagcatcagtaaagacaactccaagagccaagttttcttaaaaatgaacagtctgcaaactgatgacacagccatgtactactgtgccagaagagatggtaacaggtactatactatggactactggggtcaaggagcttcagtcaccgtctcctcaggtggtggtgggtctggcggaggtggtagtggaggtggtgggtctgaaaatgtgctcacccagtctccagcaatcatgtctgcatctccaggggaaaaggtcaccatgacctgcagggccagctcaagtgtaagttccagttacttgcactggtaccagcagaagtcaggtggctcccccaaactctggatttatagaacatccaacttggcttctggagtccctgctcgcttcagtggcagtgggtctgggacctcttactctctcacaatcagcagtgtggaggctgaagatgctgccacttattactgccagcagtacagtggttacccgctcacgttcggtgctgggaccaagctggagctgaaacgg

SEQ ID NO: 7 - protein, 39 aa, Artificial Sequence
  Exemplary amino acid sequence of a CD28 hinge
IEVMYPPPYLDNEKSNGTIIHVKGKHLCPSPLFPGPSKP

SEQ ID NO: 8 - protein, 112 aa, Artificial Sequence
  Exemplary amino acid sequence of an intracellular signalling domain of CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 41 aa, Artificial Sequence
  Exemplary amino acid sequence of the co-stimulatory intracellular signalling domain of CD28
RSKRSRLLHSDYMNMTPRRLGPTRKHYQPYAPPRDFAAYRS

SEQ ID NO: 10 - protein, 27 aa, Artificial Sequence
  Exemplary amino acid sequence of a CD28 transmembrane domain
FWVLVVVGGVLACYSLLVTVAFIIFWV

SEQ ID NO: 11 - DNA, 1380 bp, Artificial Sequence
  Exemplary coding sequence of a CAR comprising IFN-gamma scFv TII 2G1G7, a IgG4 hinge region, a CD28 transmembrane domain, a CD28 co-stimulatory domain, and a CD3 zeta signalling domain
atggctttgcctgtgactgcactgttgcttcccttggctctgcttctccatgctgctagaccgcaagttaagctggagcagtcaggacctggcctggtggcgccctcacagagcctgtccatcacttgcactgtctctgggttttcattaaccagctatggtgtacactgggttcgccagtctccaggaaagggtctggagtggctgggagtaatatgggctggtggaagcacaaattataattcggctctcatgtccagactgagcatcagtaaagacaactccaagagccaagttttcttaaaaatgaacagtctgcaaactgatgacacagccatgtactactgtgccagaagagatggtaacaggtactatactatggactactggggtcaaggagcttcagtcaccgtctcctcaggtggtggtgggtctggcggaggtggtagtggaggtggtgggtctgaaaatgtgctcacccagtctccagcaatcatgtctgcatctccaggggaaaaggtcaccatgacctgcagggccagctcaagtgtaagttccagttacttgcactggtaccagcagaagtcaggtggctcccccaaactctggatttatagaacatccaacttggcttctggagtccctgctcgcttcagtggcagtgggtctgggacctcttactctctcacaatcagcagtgtggaggctgaagatgctgccacttattactgccagcagtacagtggttacccgctcacgttcggtgctgggaccaagctggagctgaaacgggctagcgcagaaagcaagtatggtcctccctgccccccgtgcccattttgggtgctggtggtggttgggggagtcctggcttgctatagcttgctagtaacagtggcctttattattttctgggtgaggagtaagaggagcaggctcctgcacagtgactacatgaacatgactccaagacgcctaggtcccactcgcaagcattaccagccctatgccccaccacgcgacttcgcagcctatcgctccagagtgaagttcagcaggagcgcagacgctccagcttaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 12 - protein, 481 aa, Artificial Sequence
  Exemplary amino acid sequence of CAR PMC882 comprising the TNF alpha scFv, a CD28 hinge, a CD28 transmembrane domain, a CD28 co-stimulatory domain, and a CD3 zeta signalling domain (with leader sequence)
MALPVTALLLPLALLLHAARPEVQLLESGGGLVQPGGSLRLSCAASGFTFSSYAMSWVRQAPGKGLEWVSSISSTGASTTYADSVKGRFTISRDNSKNTLYLQMNSLRAEDTAVYYCAKGGAAFDYWGQGTLVTVSSGGGGSGGGGSGGGGSDIQMTQSPSSLSASVGDRVTITCRASQSISSYLNWYQQKPGKAPKLLIYSASYLQSGVPSRFSGSGSGTDFTLTISSLQPEDFATYYCQQANNAPTTFGQGTKVEIKASAIEVMYPPPYLDNEKSNGTIIHVKGKHLCPSPLFPGPSKPFWVLVVVGGVLACYSLLVTVAFIIFWVRSKRSRLLHSDYMNMTPRRLGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 1958 bp, Artificial Sequence
  Exemplary coding sequence of an NFAT-inducible expression cassette, encoding a 6(NFAT) minimal IL-2 promoter, a TGF-beta gene, a T2A peptide separating the TGF-beta and IL-10, and an IL-10 gene
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtcagaattaacagtataaattgcatctcttgttcaagagttccctatcactcgagcggtcgccaccatgccaccttctggactgcggttgcttccgttgctgttgccactgctttggctgcttgttctgacacctggcagacccgcagccggtttgtcaacatgcaaaacgatcgacatggagctggtaaagcgcaagcgcattgaggcaatcagagggcagatcttgagcaagcttcggctcgcctctcctccaagccagggcgaggtgccacccggtccgctgccggaagcggttctcgctctctataacagcactagagatagggtcgcaggtgagtctgctgaacctgaacctgaaccggaagctgactattacgctaaagaggtgacgagagttctcatggtggaaactcacaatgagatttatgataaatttaaacagtctacccacagcatttatatgttttttaacaccagtgagctccgcgaggctgtccctgaacctgtcctcctttccagggcagaattgcggcttctgaggctgaagcttaaagtagagcaacacgtggagctctaccagaaatatagtaataacagctggagatatctgtcaaaccgactgctggcgccctccgacagtccagagtggctttcctttgacgttactggcgtggtgcggcaatggctttcaagaggcggggaaatcgagggcttcaggcttagcgctcactgctcatgtgatagtagggataacacacttcaggtagacattaatgggttcacgacaggaagaagaggagatctggctacgattcatggcatgaataggcctttcctcctgcttatggctacgccattggagcgcgcgcaacatctccagtccagccgccacagacgagctctggatactaactactgcttctccagcactgagaagaactgctgcgtaagacagctttacatcgacttccgcaaggacctgggttggaagtggatacacgagcccaagggatatcatgccaacttctgcctcgggccctgtccctacatctggtcattggatacacagtacagcaaggtactcgctctgtacaaccagcacaaccctggtgcaagcgcagctccctgctgtgttccccaggctctggaaccattgcctattgtttattatgtaggacgaaaaccgaaggtcgagcagctcagtaacatgatcgttcggagctgcaagtgctctggctcaggcgagggcagaggcagcctgctgacatgtggcgacgtggaagagaaccccgggcctagtccaggtcaaggtacacagagtgagaacagttgcacacactttcccggcaacttgcctaatatgctgagggacctccgggatgctttctctcgggtaaaaactttttttcaaatgaaagaccaactcgataacctccttctgaaggagagcctcctggaggacttcaagggatatttggggtgccaagctttgtccgagatgattcagttctacttggaagaggtgatgccacaagcggagaaccaagacccggacatcaaggctcacgtcaatagcttgggagaaaacctgaagaccctgcggttgaggctgcgccgctgtcataggttccttccatgtgagaacaaatccaaagctgtggaacaggtcaaaaatgctttcaacaaactgcaggagaaaggtatatataaggccatgtctgaatttgacatatttatcaactacatagaggcgtatatgaccatgaagattcgcaat

SEQ ID NO: 14 - DNA, 4902 bp, Artificial Sequence
  Full coding sequence of the CAR construct comprising the TNF-alpha scFv, IgG4 hinge, CD28 transmembrane domain, CD28 co-stimulatory domain, CD3 zeta signalling domain with a T2A skip peptide to FOXP3 in the same cassette and a NFAT inducible
atggctttgcctgtgactgcactgttgcttcccttggctctgcttctccatgctgctagaccggactataaagatgacgatgataagggcgggtccgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtggtggtggtgggtctggcggaggtggtagtggaggtggtgggtctgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccagggcaccaaggtggaaatcaaagctagcgcagaaagcaagtatggtcctccctgccccccgtgcccattttgggtgctggtggtggttgggggagtcctggcttgctatagcttgctagtaacagtggcctttattattttctgggtgaggagtaagaggagcaggctcctgcacagtgactacatgaacatgactccaagacgcctaggtcccactcgcaagcattaccagccctatgccccaccacgcgacttcgcagcctatcgctccagagtgaagttcagcaggagcgcagacgctccagcttaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctcaggcgagggcagaggcagcctgctgacatgtggcgacgtggaagagaaccccgggcctatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttaatagacgcgtgggttaggggaggcgcttttcccaaggcagtctggagcatgcgctaagcgatcgcgcccgctgggcacttggcgctacacaagtggcctctggcctcgcacacattccaggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtcagaattaacagtataaattgcatctcttgttcaagagttccctatcactcgagcggtcgccaccatgccaccttctggactgcggttgcttccgttgctgttgccactgctttggctgcttgttctgacacctggcagacccgcagccggtttgtcaacatgcaaaacgatcgacatggagctggtaaagcgcaagcgcattgaggcaatcagagggcagatcttgagcaagcttcggctcgcctctcctccaagccagggcgaggtgccacccggtccgctgccggaagcggttctcgctctctataacagcactagagatagggtcgcaggtgagtctgctgaacctgaacctgaaccggaagctgactattacgctaaagaggtgacgagagttctcatggtggaaactcacaatgagatttatgataaatttaaacagtctacccacagcatttatatgttttttaacaccagtgagctccgcgaggctgtccctgaacctgtcctcctttccagggcagaattgcggcttctgaggctgaagcttaaagtagagcaacacgtggagctctaccagaaatatagtaataacagctggagatatctgtcaaaccgactgctggcgccctccgacagtccagagtggctttcctttgacgttactggcgtggtgcggcaatggctttcaagaggcggggaaatcgagggcttcaggcttagcgctcactgctcatgtgatagtagggataacacacttcaggtagacattaatgggttcacgacaggaagaagaggagatctggctacgattcatggcatgaataggcctttcctcctgcttatggctacgccattggagcgcgcgcaacatctccagtccagccgccacagacgagctctggatactaactactgcttctccagcactgagaagaactgctgcgtaagacagctttacatcgacttccgcaaggacctgggttggaagtggatacacgagcccaagggatatcatgccaacttctgcctcgggccctgtccctacatctggtcattggatacacagtacagcaaggtactcgctctgtacaaccagcacaaccctggtgcaagcgcagctccctgctgtgttccccaggctctggaaccattgcctattgtttattatgtaggacgaaaaccgaaggtcgagcagctcagtaacatgatcgttcggagctgcaagtgctctggctcaggcgagggcagaggcagcctgctgacatgtggcgacgtggaagagaaccccgggcctatgcacagctcagcactgctctgttgcctggtcctcctgactggggtgagggccagtccaggtcaaggtacacagagtgagaacagttgcacacactttcccggcaacttgcctaatatgctgagggacctccgggatgctttctctcgggtaaaaactttttttcaaatgaaagaccaactcgataacctccttctgaaggagagcctcctggaggacttcaagggatatttggggtgccaagctttgtccgagatgattcagttctacttggaagaggtgatgccacaagcggagaaccaagacccggacatcaaggctcacgtcaatagcttgggagaaaacctgaagaccctgcggttgaggctgcgccgctgtcataggttccttccatgtgagaacaaatccaaagctgtggaacaggtcaaaaatgctttcaacaaactgcaggagaaaggtatatataaggccatgtctgaatttgacatatttatcaactacatagaggcgtatatgaccatgaagattcgcaattga

SEQ ID NO: 15 - DNA, 4815 bp, Artificial Sequence
  Full sequence of a CAR construct comprising of IFN-gamma scFv TII 2G1G7, an IgG4 hinge, CD28 transmembrane domain, CD28 co-stimulatory domain, CD3 zeta signalling domain with a T2A skip peptide to FOXP3 in the same cassette and a NFAT inducible
atggctttgcctgtgactgcactgttgcttcccttggctctgcttctccatgctgctagaccgcaagttaagctggagcagtcaggacctggcctggtggcgccctcacagagcctgtccatcacttgcactgtctctgggttttcattaaccagctatggtgtacactgggttcgccagtctccaggaaagggtctggagtggctgggagtaatatgggctggtggaagcacaaattataattcggctctcatgtccagactgagcatcagtaaagacaactccaagagccaagttttcttaaaaatgaacagtctgcaaactgatgacacagccatgtactactgtgccagaagagatggtaacaggtactatactatggactactggggtcaaggagcttcagtcaccgtctcctcaggtggtggtgggtctggcggaggtggtagtggaggtggtgggtctgaaaatgtgctcacccagtctccagcaatcatgtctgcatctccaggggaaaaggtcaccatgacctgcagggccagctcaagtgtaagttccagttacttgcactggtaccagcagaagtcaggtggctcccccaaactctggatttatagaacatccaacttggcttctggagtccctgctcgcttcagtggcagtgggtctgggacctcttactctctcacaatcagcagtgtggaggctgaagatgctgccacttattactgccagcagtacagtggttacccgctcacgttcggtgctgggaccaagctggagctgaaacgggctagcgcagaaagcaagtatggtcctccctgccccccgtgcccattttgggtgctggtggtggttgggggagtcctggcttgctatagcttgctagtaacagtggcctttattattttctgggtgaggagtaagaggagcaggctcctgcacagtgactacatgaacatgactccaagacgcctaggtcccactcgcaagcattaccagccctatgccccaccacgcgacttcgcagcctatcgctccagagtgaagttcagcaggagcgcagacgctccagcttaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctcaggcgagggcagaggcagcctgctgacatgtggcgacgtggaagagaaccccgggcctatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttaatagacgcgtgggttaggggaggcgcttttcccaaggcagtctggagcatgcgctaattagctagccccgctgggcacttggcgctacacaagtggcctctggcctcgcacacattccaggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtcagaattaacagtataaattgcatctcttgttcaagagttccctatcactcgagcggtcgccaccatgccaccttctggactgcggttgcttccgttgctgttgccactgctttggctgcttgttctgacacctggcagacccgcagccggtttgtcaacatgcaaaacgatcgacatggagctggtaaagcgcaagcgcattgaggcaatcagagggcagatcttgagcaagcttcggctcgcctctcctccaagccagggcgaggtgccacccggtccgctgccggaagcggttctcgctctctataacagcactagagatagggtcgcaggtgagtctgctgaacctgaacctgaaccggaagctgactattacgctaaagaggtgacgagagttctcatggtggaaactcacaatgagatttatgataaatttaaacagtctacccacagcatttatatgttttttaacaccagtgagctccgcgaggctgtccctgaacctgtcctcctttccagggcagaattgcggcttctgaggctgaagcttaaagtagagcaacacgtggagctctaccagaaatatagtaataacagctggagatatctgtcaaaccgactgctggcgccctccgacagtccagagtggctttcctttgacgttactggcgtggtgcggcaatggctttcaagaggcggggaaatcgagggcttcaggcttagcgctcactgctcatgtgatagtagggataacacacttcaggtagacattaatgggttcacgacaggaagaagaggagatctggctacgattcatggcatgaataggcctttcctcctgcttatggctacgccattggagcgcgcgcaacatctccagtccagccgccacagacgagctctggatactaactactgcttctccagcactgagaagaactgctgcgtaagacagctttacatcgacttccgcaaggacctgggttggaagtggatacacgagcccaagggatatcatgccaacttctgcctcgggccctgtccctacatctggtcattggatacacagtacagcaaggtactcgctctgtacaaccagcacaaccctggtgcaagcgcagctccctgctgtgttccccaggctctggaaccattgcctattgtttattatgtaggacgaaaaccgaaggtcgagcagctcagtaacatgatcgttcggagctgcaagtgctctggctcaggcgagggcagaggcagcctgctgacatgtggcgacgtggaagagaaccccgggcctagtccaggtcaaggtacacagagtgagaacagttgcacacactttcccggcaacttgcctaatatgctgagggacctccgggatgctttctctcgggtaaaaactttttttcaaatgaaagaccaactcgataacctccttctgaaggagagcctcctggaggacttcaagggatatttggggtgccaagctttgtccgagatgattcagttctacttggaagaggtgatgccacaagcggagaaccaagacccggacatcaaggctcacgtcaatagcttgggagaaaacctgaagaccctgcggttgaggctgcgccgctgtcataggttccttccatgtgagaacaaatccaaagctgtggaacaggtcaaaaatgctttcaacaaactgcaggagaaaggtatatataaggccatgtctgaatttgacatatttatcaactacatagaggcgtatatgaccatgaagattcgcaattga

SEQ ID NO: 16 - protein, 160 aa, Artificial Sequence
  Exemplary amino acid sequence of IL-10
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 17 - protein, 389 aa, Artificial Sequence
  Exemplary amino acid sequence of TGF-beta
PPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 18 - protein, 431 aa, Artificial Sequence
  Exemplary amino acid sequence of FOXP3
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 19 - protein, 116 aa, Artificial Sequence
  anti-TNF-alpha scFv clone P3H2 VH
EVQLLESGGGLVQPGGSLRLSCAASGFTFSSYAMSWVRQAPGKGLEWVSSISSTGASTTYADSVKGRFTISRDNSKNTLYLQMNSLRAEDTAVYYCAKGGAAFDYWGQGTLVTVSS

SEQ ID NO: 20 - protein, 107 aa, Artificial Sequence
  anti-TNF-alpha scFv clone P3H2 VL
DIQMTQSPSSLSASVGDRVTITCRASQSISSYLNWYQQKPGKAPKLLIYSASYLQSGVPSRFSGSGSGTDFTLTISSLQPEDFATYYCQQANNAPTTFGQGTKVEIK

SEQ ID NO: 21 - protein, 5 aa, Artificial Sequence
  anti-TNF-alpha scFv clone P3H2 & Anti-IFNy NovImmune Clone A6 - Emapalumab - CDRH1 (Kabat)
SYAMS

SEQ ID NO: 22 - protein, 10 aa, Artificial Sequence
  anti-TNF-alpha scFv clone P3H2 & Anti-IFNy NovImmune Clone A6 - Emapalumab - CDRH1 (AbM)
GFTFSSYAMS

SEQ ID NO: 23 - protein, 8 aa, Artificial Sequence
  anti-TNF-alpha scFv clone P3H2  & Anti-IFNy NovImmune Clone A6 - Emapalumab - CDRH1 (IMGT)
GFTFSSYA

SEQ ID NO: 24 - protein, 17 aa, Artificial Sequence
  anti-TNF-alpha scFv clone P3H2 - CDRH2 (Kabat)
SISSTGASTTYADSVKG

SEQ ID NO: 25 - protein, 10 aa, Artificial Sequence
  anti-TNF-alpha scFv clone P3H2 - CDRH2 (AbM)
SISSTGASTT

SEQ ID NO: 26 - protein, 8 aa, Artificial Sequence
  anti-TNF-alpha scFv clone P3H2 - CDRH2 (IMGT)
ISSTGAST

SEQ ID NO: 27 - protein, 7 aa, Artificial Sequence
  anti-TNF-alpha scFv clone P3H2 - CDRH3 (Kabat, AbM)
GGAAFDY

SEQ ID NO: 28 - protein, 9 aa, Artificial Sequence
  anti-TNF-alpha scFv clone P3H2 - CDRH3 (IMGT)
AKGGAAFDY

SEQ ID NO: 29 - protein, 11 aa, Artificial Sequence
  anti-TNF-alpha scFv clone P3H2 - CDRL1 (Kabat, AbM)
RASQSISSYLN

SEQ ID NO: 30 - protein, 6 aa, Artificial Sequence
  anti-TNF-alpha scFv clone P3H2 - CDRL1 (IMGT)
QSISSY

SEQ ID NO: 31 - protein, 7 aa, Artificial Sequence
  anti-TNF-alpha scFv clone P3H2 - CDRL2 (Kabat, AbM)
SASYLQS

SEQ ID NO: 32 - protein, 2 aa, Artificial Sequence
  anti-TNF-alpha scFvclone P3H2 - CDRL2 (IMGT)
SA

SEQ ID NO: 33 - protein, 9 aa, Artificial Sequence
  anti-TNF-alpha scFv clone P3H2 - CDRL3 (Kabat, AbM, IMGT)
QQANNAPTT

SEQ ID NO: 34 - protein, 118 aa, Artificial Sequence
  IFN-gamma scFv TII 6AE52 VH
EVKLEQSGTVLARPGASVKMSCKASGYTFTNYWMHWIRQRPGQGLEWIGAIYPGISDTSYNQKFKVKAKLTAVTSTSTAYMELSSLTNEDSAVYYCTRDEDFDGFAYWGQGTLVTVSA

SEQ ID NO: 35 - protein, 113 aa, Artificial Sequence
  IFN-gamma scFv TII 6AE52 VL
DVLMTQTPLSLPVSLGDQASISCRSSQSIVHSNGNTYLDWYLQKPGQSPKLLIYKVSNRFSGVPDRFSGSGSGTDFTLRISRVEAEDLGVYYCFRGSHAPYTFGGGTKLEIKR

SEQ ID NO: 36 - protein, 5 aa, Artificial Sequence
  IFN-gamma scFv TII 6AE52 - CDRH1 (Kabat)
NYWMH

SEQ ID NO: 37 - protein, 10 aa, Artificial Sequence
  IFN-gamma scFv TII 6AE52 - CDRH1 (AbM)
GYTFTNYWMH

SEQ ID NO: 38 - protein, 8 aa, Artificial Sequence
  IFN-gamma scFv TII 6AE52 - CDRH1 (IMGT)
GYTFTNYW

SEQ ID NO: 39 - protein, 17 aa, Artificial Sequence
  IFN-gamma scFv TII 6AE52 - CDRH2 (Kabat)
AIYPGISDTSYNQKFKV

SEQ ID NO: 40 - protein, 10 aa, Artificial Sequence
  IFN-gamma scFv TII 6AE52 - CDRH2 (AbM)
AIYPGISDTS

SEQ ID NO: 41 - protein, 8 aa, Artificial Sequence
  IFN-gamma scFv TII 6AE52 - CDRH2 (IMGT)
IYPGISDT

SEQ ID NO: 42 - protein, 9 aa, Artificial Sequence
  IFN-gamma scFv TII 6AE52 - CDRH3 (Kabat, AbM)
DEDFDGFAY

SEQ ID NO: 43 - protein, 11 aa, Artificial Sequence
  IFN-gamma scFv TII 6AE52 - CDRH3 (IMGT)
TRDEDFDGFAY

SEQ ID NO: 44 - protein, 16 aa, Artificial Sequence
  IFN-gamma scFv TII 6AE52 - CDRL1 (Kabat, AbM)
RSSQSIVHSNGNTYLD

SEQ ID NO: 45 - protein, 11 aa, Artificial Sequence
  IFN-gamma scFv TII 6AE52 - CDRL1 (IMGT)
QSIVHSNGNTY

SEQ ID NO: 46 - protein, 7 aa, Artificial Sequence
  IFN-gamma scFv TII 6AE52 - CDRL2 (Kabat, AbM)
KVSNRFS

SEQ ID NO: 47 - protein, 2 aa, Artificial Sequence
  IFN-gamma scFv TII 6AE52 & Anti-IL17A Clone Mab126 - Ixekizumab - CDRL2 (IMGT)
KV

SEQ ID NO: 48 - protein, 9 aa, Artificial Sequence
  IFN-gamma scFv TII 6AE52 - CDRL3 (Kabat, AbM, IMGT)
FRGSHAPYT

SEQ ID NO: 49 - protein, 119 aa, Artificial Sequence
  IFN-gamma scFv TII 2G1G7 VH
QVKLEQSGPGLVAPSQSLSITCTVSGFSLTSYGVHWVRQSPGKGLEWLGVIWAGGSTNYNSALMSRLSISKDNSKSQVFLKMNSLQTDDTAMYYCARRDGNRYYTMDYWGQGASVTVSS

SEQ ID NO: 50 - protein, 109 aa, Artificial Sequence
  IFN-gamma scFv TII 2G1G7 VL
ENVLTQSPAIMSASPGEKVTMTCRASSSVSSSYLHWYQQKSGGSPKLWIYRTSNLASGVPARFSGSGSGTSYSLTISSVEAEDAATYYCQQYSGYPLTFGAGTKLELKR

SEQ ID NO: 51 - protein, 5 aa, Artificial Sequence
  IFN-gamma scFv TII 2G1G7 - CDRH1 (Kabat)
SYGVH

SEQ ID NO: 52 - protein, 10 aa, Artificial Sequence
  IFN-gamma scFv TII 2G1G7 - CDRH1 (AbM)
GFSLTSYGVH

SEQ ID NO: 53 - protein, 8 aa, Artificial Sequence
  IFN-gamma scFv TII 2G1G7 - CDRH1 (IMGT)
GFSLTSYG

SEQ ID NO: 54 - protein, 16 aa, Artificial Sequence
  IFN-gamma scFv TII 2G1G7 - CDRH2 (Kabat)
VIWAGGSTNYNSALMS

SEQ ID NO: 55 - protein, 9 aa, Artificial Sequence
  IFN-gamma scFv TII 2G1G7 - CDRH2 (AbM)
VIWAGGSTN

SEQ ID NO: 56 - protein, 7 aa, Artificial Sequence
  IFN-gamma scFv TII 2G1G7 - CDRH2 (IMGT)
IWAGGST

SEQ ID NO: 57 - protein, 11 aa, Artificial Sequence
  IFN-gamma scFv TII 2G1G7 - CDRH3 (Kabat, AbM)
RDGNRYYTMDY

SEQ ID NO: 58 - protein, 13 aa, Artificial Sequence
  IFN-gamma scFv TII 2G1G7 - CDRH3 (IMGT)
ARRDGNRYYTMDY

SEQ ID NO: 59 - protein, 12 aa, Artificial Sequence
  IFN-gamma scFv TII 2G1G7 - CDRL1 (Kabat, AbM)
RASSSVSSSYLH

SEQ ID NO: 60 - protein, 7 aa, Artificial Sequence
  IFN-gamma scFv TII 2G1G7 - CDRL1 (IMGT)
SSVSSSY

SEQ ID NO: 61 - protein, 7 aa, Artificial Sequence
  IFN-gamma scFv TII 2G1G7 - CDRL2 (Kabat, AbM)
RTSNLAS

SEQ ID NO: 62 - protein, 2 aa, Artificial Sequence
  IFN-gamma scFv TII 2G1G7 - CDRL2 (IMGT)
RT

SEQ ID NO: 63 - protein, 9 aa, Artificial Sequence
  IFN-gamma scFv TII 2G1G7 - CDRL3 (Kabat, AbM, IMGT)
QQYSGYPLT

SEQ ID NO: 64 - protein, 243 aa, Artificial Sequence
  Exemplary amino acid sequence of anti-TNFa scFv clone 2DE7
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIK

SEQ ID NO: 65 - protein, 121 aa, Artificial Sequence [sequence omitted]

SEQ ID NO: 66 - protein, 107 aa, Artificial Sequence [sequence omitted]

SEQ ID NO: 67 - protein, 5 aa, Artificial Sequence
  Anti-TNFa scFv clone Humira D2E7  & Anti-TNFa scFv clone 2SD4- CDRH1 (Kabat)
DYAMH

SEQ ID NO: 68 - protein, 10 aa, Artificial Sequence
  Anti-TNFa scFv clone Humira D2E7  & Anti-TNFa scFv clone 2SD4- CDRH1 (AbM)
GFTFDDYAMH

SEQ ID NO: 69 - protein, 8 aa, Artificial Sequence
  Anti-TNFa scFv clone Humira D2E7 & Anti-TNFa scFv clone 2SD4 - CDRH1 (IMGT)
GFTFDDYA

SEQ ID NO: 70 - protein, 17 aa, Artificial Sequence
  Anti-TNFa scFv clone Humira D2E7  & Anti-TNFa scFv clone 2SD4- CDRH2 (Kabat)
AITWNSGHIDYADSVEG

SEQ ID NO: 71 - protein, 10 aa, Artificial Sequence
  Anti-TNFa scFv clone Humira D2E7 & Anti-TNFa scFv clone 2SD4 - CDRH2 (AbM)
AITWNSGHID

SEQ ID NO: 72 - protein, 8 aa, Artificial Sequence
  Anti-TNFa scFv clone Humira D2E7  & Anti-TNFa scFv clone 2SD4 - CDRH2 (IMGT)
ITWNSGHI

SEQ ID NO: 73 - protein, 12 aa, Artificial Sequence
  Anti-TNFa scFv clone Humira D2E7  - CDRH3 (Kabat, AbM)
VSYLSTASSLDY

SEQ ID NO: 74 - protein, 14 aa, Artificial Sequence
  Anti-TNFa scFv clone Humira D2E7  - CDRH3 (IMGT)
AKVSYLSTASSLDY

SEQ ID NO: 75 - protein, 11 aa, Artificial Sequence
  Anti-TNFa scFv clone Humira D2E7 & Anti-TNFa scFv clone 2SD4 - CDRL1 (Kabat, AbM)
RASQGIRNYLA

SEQ ID NO: 76 - protein, 6 aa, Artificial Sequence
  Anti-TNFa scFv clone Humira D2E7 & Anti-TNFa scFv clone 2SD4 - CDRL1 (IMGT)
QGIRNY

SEQ ID NO: 77 - protein, 7 aa, Artificial Sequence
  Anti-TNFa scFv clone Humira D2E7 & Anti-TNFa scFv clone 2SD4 - CDRL2 (Kabat, AbM)
AASTLQS

SEQ ID NO: 78 - protein, 2 aa, Artificial Sequence
  Anti-TNFa scFv clone Humira D2E7 & Anti-TNFa scFv clone 2SD4 - CDRL2 (IMGT)
AA

SEQ ID NO: 79 - protein, 9 aa, Artificial Sequence
  Anti-TNFa scFv clone Humira D2E7  - CDRL3 (Kabat, AbM, IMGT)
QRYNRAPYT

SEQ ID NO: 80 - protein, 120 aa, Artificial Sequence
  Anti-TNFa scFv clone 2SD4 VH
QVQLVESGGGLVQPGRSLRLSCAASGFTFDDYAMHWVRQAPGKGLDWVSAITWNSGHIDYADSVEGRFAVSRDNAKNALYLQMNSLRPEDTAVYYCTKASYLSTSSLDNWGQGTLVTVSS

SEQ ID NO: 81 - protein, 107 aa, Artificial Sequence
  Anti-TNFa scFv clone 2SD4 VL
DIQMTQSPSSLSASIGDRVTITVRASQGIRNYLAWYQQKPGKAPKLLIYAASTLQSGVPSRFSGSGSGTDFTLTISSLQPEDVATYYCQKYNSAPYAFGQGTKVEIK

SEQ ID NO: 82 - protein, 11 aa, Artificial Sequence
  Anti-TNFa scFv clone 2SD4 VL - CDRH3 (Kabat, AbM)
ASYLSTSSLDN

SEQ ID NO: 83 - protein, 13 aa, Artificial Sequence
  Anti-TNFa scFv clone 2SD4 VL - CDRH3 (IMGT)
TKASYLSTSSLDN

SEQ ID NO: 84 - protein, 9 aa, Artificial Sequence
  Anti-TNFa scFv clone 2SD4 VL - CDRL3 (Kabat, AbM, IMGT)
QKYNSAPYA

SEQ ID NO: 85 - protein, 119 aa, Artificial Sequence
  Anti-TNFa scFv clone cA2 - Infliximab VH
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVS

SEQ ID NO: 86 - protein, 107 aa, Artificial Sequence
  Anti-TNFa scFv clone cA2 - Infliximab VL
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 87 - protein, 5 aa, Artificial Sequence
  Anti-TNFa scFv clone cA2 - Infliximab  - CDRH1 (Kabat)
NHWMN

SEQ ID NO: 88 - protein, 10 aa, Artificial Sequence
  Anti-TNFa scFv clone cA2 - Infliximab  - CDRH1 (AbM)
GFIFSNHWMN

SEQ ID NO: 89 - protein, 8 aa, Artificial Sequence
  Anti-TNFa scFv clone cA2 - Infliximab  - CDRH1 (IMGT)
GFIFSNHW

SEQ ID NO: 90 - protein, 19 aa, Artificial Sequence
  Anti-TNFa scFv clone cA2 - Infliximab  - CDRH2 (Kabat)
EIRSKSINSATHYAESVKG

SEQ ID NO: 91 - protein, 12 aa, Artificial Sequence
  Anti-TNFa scFv clone cA2 - Infliximab  - CDRH2 (AbM)
EIRSKSINSATH

SEQ ID NO: 92 - protein, 10 aa, Artificial Sequence
  Anti-TNFa scFv clone cA2 - Infliximab  - CDRH2 (IMGT)
IRSKSINSAT

SEQ ID NO: 93 - protein, 9 aa, Artificial Sequence
  Anti-TNFa scFv clone cA2 - Infliximab  - CDRH3 (Kabat, AbM)
NYYGSTYDY

SEQ ID NO: 94 - protein, 11 aa, Artificial Sequence
  Anti-TNFa scFv clone cA2 - Infliximab  - CDRH3 (IMGT)
SRNYYGSTYDY

SEQ ID NO: 95 - protein, 11 aa, Artificial Sequence
  Anti-TNFa scFv clone cA2 - Infliximab  - CDRL1 (Kabat, AbM)
RASQFVGSSIH

SEQ ID NO: 96 - protein, 6 aa, Artificial Sequence
  Anti-TNFa scFv clone cA2 - Infliximab  - CDRL1 (IMGT)
QFVGSS

SEQ ID NO: 97 - protein, 7 aa, Artificial Sequence
  Anti-TNFa scFv clone cA2 - Infliximab  - CDRL2 (Kabat, AbM)
YASESMS

SEQ ID NO: 98 - protein, 2 aa, Artificial Sequence
  Anti-TNFa scFv clone cA2 - Infliximab  - CDRL2 (IMGT)
YA

SEQ ID NO: 99 - protein, 9 aa, Artificial Sequence
  Anti-TNFa scFv clone cA2 - Infliximab  - CDRL3 (Kabat, AbM, IMGT)
QQSHSWPFT

SEQ ID NO: 100 - protein, 123 aa, Artificial Sequence
  Anti-IFNy NovImmune Clone A6 - Emapalumab VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGSSGWYVPHWFDPWGRGTLVTVSS

SEQ ID NO: 101 - protein, 112 aa, Artificial Sequence
  Anti-IFNy NovImmune Clone A6 - Emapalumab VL
NFMLTQPHSVSESPGKTVTISCTRSSGSIVSNYVQWYQQRPGSAPTTVIYEDNRRPSGVPDRFSGSIDSSSNTASLTISGLEAEDEADYYCQSYDGSNRWMFGGGTKLTVLG

SEQ ID NO: 102 - protein, 17 aa, Artificial Sequence
  Anti-IFNy NovImmune Clone A6 - Emapalumab  - CDRH2 (Kabat)
AISGSGGSTYYADSVKG

SEQ ID NO: 103 - protein, 10 aa, Artificial Sequence
  Anti-IFNy NovImmune Clone A6 - Emapalumab  - CDRH2 (AbM)
AISGSGGSTY

SEQ ID NO: 104 - protein, 8 aa, Artificial Sequence
  Anti-IFNy NovImmune Clone A6 - Emapalumab  - CDRH2 (IMGT)
ISGSGGST

SEQ ID NO: 105 - protein, 14 aa, Artificial Sequence
  Anti-IFNy NovImmune Clone A6 - Emapalumab  - CDRH3 (Kabat, AbM)
DGSSGWYVPHWFDP

SEQ ID NO: 106 - protein, 16 aa, Artificial Sequence
  Anti-IFNy NovImmune Clone A6 - Emapalumab  - CDRH3 (IMGT)
AKDGSSGWYVPHWFDP

SEQ ID NO: 107 - protein, 13 aa, Artificial Sequence
  Anti-IFNy NovImmune Clone A6 - Emapalumab  - CDRL1 (Kabat, AbM)
TRSSGSIVSNYVQ

SEQ ID NO: 108 - protein, 8 aa, Artificial Sequence
  Anti-IFNy NovImmune Clone A6 - Emapalumab  - CDRL1 (IMGT)
SGSIVSNY

SEQ ID NO: 109 - protein, 7 aa, Artificial Sequence
  Anti-IFNy NovImmune Clone A6 - Emapalumab  - CDRL2 (Kabat, AbM)
EDNRRPS

SEQ ID NO: 110 - protein, 2 aa, Artificial Sequence
  Anti-IFNy NovImmune Clone A6 - Emapalumab  - CDRL2 (IMGT)
ED

SEQ ID NO: 111 - protein, 10 aa, Artificial Sequence
  Anti-IFNy NovImmune Clone A6 - Emapalumab  - CDRL3 (Kabat, AbM, IMGT)
QSYDGSNRWM

SEQ ID NO: 112 - protein, 117 aa, Artificial Sequence
  Anti-IFNy HuZAF - Fontolizumab VH
QVQLVQSGAELKKPGSSVKVSCKASGYIFTSSWINWVQAPGQGLEWIGRIDPSDGEVHYNQDFKDKATLTTVDKSTNTAYMELSSLRSEDTAVYYCARGFLPWFADWGQGTLVTVSS

SEQ ID NO: 113 - protein, 108 aa, Artificial Sequence
  Anti-IFNy HuZAF - Fontolizumab VL
DIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKR

SEQ ID NO: 114 - protein, 5 aa, Artificial Sequence
  Anti-IFNy HuZAF - Fontolizumab - CDRH1 (Kabat)
SSWIN

SEQ ID NO: 115 - protein, 10 aa, Artificial Sequence
  Anti-IFNy HuZAF - Fontolizumab - CDRH1 (AbM)
GYIFTSSWIN

SEQ ID NO: 116 - protein, 8 aa, Artificial Sequence
  Anti-IFNy HuZAF - Fontolizumab - CDRH1 (IMGT)
GYIFTSSW

SEQ ID NO: 117 - protein, 17 aa, Artificial Sequence
  Anti-IFNy HuZAF - Fontolizumab - CDRH2 (Kabat)
RIDPSDGEVHYNQDFKD

SEQ ID NO: 118 - protein, 8 aa, Artificial Sequence
  Anti-IFNy HuZAF - Fontolizumab - CDRH2 (AbM)
IDPSDGEV

SEQ ID NO: 119 - protein, 10 aa, Artificial Sequence
  Anti-IFNy HuZAF - Fontolizumab - CDRH2 (IMGT)
RIDPSDGEVH

SEQ ID NO: 120 - protein, 8 aa, Artificial Sequence
  Anti-IFNy HuZAF - Fontolizumab - CDRH3 (Kabat, AbM)
GFLPWFAD

SEQ ID NO: 121 - protein, 10 aa, Artificial Sequence
  Anti-IFNy HuZAF - Fontolizumab - CDRH3 (IMGT)
ARGFLPWFAD

SEQ ID NO: 122 - protein, 11 aa, Artificial Sequence
  Anti-IFNy HuZAF - Fontolizumab - CDRL1 (Kabat, AbM)
KASENVDTYVS

SEQ ID NO: 123 - protein, 6 aa, Artificial Sequence
  Anti-IFNy HuZAF - Fontolizumab - CDRL1 (IMGT)
ENVDTY

SEQ ID NO: 124 - protein, 7 aa, Artificial Sequence
  Anti-IFNy HuZAF - Fontolizumab - CDRL2 (Kabat, AbM)
GASNRYT

SEQ ID NO: 125 - protein, 2 aa, Artificial Sequence
  Anti-IFNy HuZAF - Fontolizumab & Anti-IL17A Clone AIN457 - Secukinumab - CDRL2 (IMGT)
GA

SEQ ID NO: 126 - protein, 9 aa, Artificial Sequence
  Anti-IFNy HuZAF - Fontolizumab - CDRL3 (Kabat, AbM, IMGT)
GQSYNYPFT

SEQ ID NO: 127 - protein, 127 aa, Artificial Sequence [sequence omitted]

SEQ ID NO: 128 - protein, 109 aa, Artificial Sequence
  Anti-IL17A Clone AIN457 - Secukinumab VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 129 - protein, 5 aa, Artificial Sequence
  Anti-IL17A Clone AIN457 - Secukinumab - CDRH1 (Kabat)
NYWMN

SEQ ID NO: 130 - protein, 10 aa, Artificial Sequence
  Anti-IL17A Clone AIN457 - Secukinumab - CDRH1 (AbM)
GFTFSNYWMN

SEQ ID NO: 131 - protein, 8 aa, Artificial Sequence
  Anti-IL17A Clone AIN457 - Secukinumab - CDRH1 (IMGT)
GFTFSNYW

SEQ ID NO: 132 - protein, 17 aa, Artificial Sequence
  Anti-IL17A Clone AIN457 - Secukinumab - CDRH2 (Kabat)
AINQDGSEKYYVGSVKG

SEQ ID NO: 133 - protein, 10 aa, Artificial Sequence
  Anti-IL17A Clone AIN457 - Secukinumab - CDRH2 (AbM)
AINQDGSEKY

SEQ ID NO: 134 - protein, 8 aa, Artificial Sequence
  Anti-IL17A Clone AIN457 - Secukinumab - CDRH2 (IMGT)
INQDGSEK

SEQ ID NO: 135 - protein, 18 aa, Artificial Sequence
  Anti-IL17A Clone AIN457 - Secukinumab - CDRH3 (Kabat, AbM)
DYYDILTDYYIHYWYFDL

SEQ ID NO: 136 - protein, 20 aa, Artificial Sequence [sequence omitted]

SEQ ID NO: 137 - protein, 12 aa, Artificial Sequence
  Anti-IL17A Clone AIN457 - Secukinumab - CDRL1 (Kabat, AbM)
RASQSVSSSYLA

SEQ ID NO: 138 - protein, 7 aa, Artificial Sequence
  Anti-IL17A Clone AIN457 - Secukinumab - CDRL1 (IMGT)
QSVSSSY

SEQ ID NO: 139 - protein, 7 aa, Artificial Sequence
  Anti-IL17A Clone AIN457 - Secukinumab - CDRL2 (Kabat, AbM)
GASSRAT

SEQ ID NO: 140 - protein, 9 aa, Artificial Sequence
  Anti-IL17A Clone AIN457 - Secukinumab - CDRL3 (Kabat, AbM, IMGT)
QQYGSSPCT

SEQ ID NO: 141 - protein, 119 aa, Artificial Sequence
  Anti-IL17A Clone Mab126 - Ixekizumab VH
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSS

SEQ ID NO: 142 - protein, 112 aa, Artificial Sequence
  Anti-IL17A Clone Mab126 - Ixekizumab VL
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIK

SEQ ID NO: 143 - protein, 5 aa, Artificial Sequence
  Anti-IL17A Clone Mab126 - Ixekizumab - CDRH1 (Kabat)
DYHIH

SEQ ID NO: 144 - protein, 10 aa, Artificial Sequence
  Anti-IL17A Clone Mab126 - Ixekizumab - CDRH1 (AbM)
GYSFTDYHIH

SEQ ID NO: 145 - protein, 8 aa, Artificial Sequence
  Anti-IL17A Clone Mab126 - Ixekizumab - CDRH1 (IMGT)
GYSFTDYH

SEQ ID NO: 146 - protein, 17 aa, Artificial Sequence
  Anti-IL17A Clone Mab126 - Ixekizumab - CDRH2 (Kabat)
VINPMYGTTDYNQRFKG

SEQ ID NO: 147 - protein, 10 aa, Artificial Sequence
  Anti-IL17A Clone Mab126 - Ixekizumab - CDRH2 (AbM)
VINPMYGTTD

SEQ ID NO: 148 - protein, 8 aa, Artificial Sequence
  Anti-IL17A Clone Mab126 - Ixekizumab - CDRH2 (IMGT)
INPMYGTT

SEQ ID NO: 149 - protein, 10 aa, Artificial Sequence
  Anti-IL17A Clone Mab126 - Ixekizumab - CDRH3 (Kabat, AbM)
YDYFTGTGVY

SEQ ID NO: 150 - protein, 12 aa, Artificial Sequence
  Anti-IL17A Clone Mab126 - Ixekizumab - CDRH3 (IMGT)
ARYDYFTGTGVY

SEQ ID NO: 151 - protein, 16 aa, Artificial Sequence
  Anti-IL17A Clone Mab126 - Ixekizumab - CDRL1 (Kabat, AbM)
RSSRSLVHSRGNTYLH

SEQ ID NO: 152 - protein, 11 aa, Artificial Sequence
  Anti-IL17A Clone Mab126 - Ixekizumab - CDRL1 (IMGT)
RSLVHSRGNTY

SEQ ID NO: 153 - protein, 7 aa, Artificial Sequence
  Anti-IL17A Clone Mab126 - Ixekizumab - CDRL2 (Kabat, AbM)
KVSNRFI

SEQ ID NO: 154 - protein, 9 aa, Artificial Sequence
  Anti-IL17A Clone Mab126 - Ixekizumab - CDRL3 (Kabat, AbM, IMGT)
SQSTHLPFT

SEQ ID NO: 155 - protein, 24 aa, Artificial Sequence
  Exemplary amino acid sequence of a CD8 transmembrane domain
IYIWAPLAGTCGVLLLSLVITLYC

SEQ ID NO: 156 - protein, 3 aa, Artificial Sequence
  Exemplary amino acid sequence of a IgG4 hinge
  misc_feature (1)..(3): Xaa can be any naturally occurring amino acid
XXX

SEQ ID NO: 157 - protein, 282 aa, Artificial Sequence
  Exemplary amino acid sequence of a IgD hinge
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 158 - protein, 42 aa, Artificial Sequence
  Exemplary amino acid sequence of a 4-1BB co-stimulatrory intracellular signalling domain
KRGRKKLLYIFKQPFMRPVQTTQEEDGCSCRFPEEEEGGCEL

SEQ ID NO: 159 - protein, 48 aa, Artificial Sequence
  Exmplary amino acid sequence of a CD27 co-stimulatory intracellular signalling domain
QRRKYRSNKGESPVEPAEPCRYSCPREEEGSTIPIQEDYRKPEPACSP

SEQ ID NO: 160 - protein, 459 aa, Artificial Sequence
  Exemplary amino acid sequence of a CAR comprising IFN-gamma scFv TII 2G1G7, a IgG4 hinge region, a CD28 transmembrane domain, a CD28 co-stimulatory domain, and a CD3 zeta signalling domain (with leader sequence)
MALPVTALLLPLALLLHAARPQVKLEQSGPGLVAPSQSLSITCTVSGFSLTSYGVHWVRQSPGKGLEWLGVIWAGGSTNYNSALMSRLSISKDNSKSQVFLKMNSLQTDDTAMYYCARRDGNRYYTMDYWGQGASVTVSSGGGGSGGGGSGGGGSENVLTQSPAIMSASPGEKVTMTCRASSSVSSSYLHWYQQKSGGSPKLWIYRTSNLASGVPARFSGSGSGTSYSLTISSVEAEDAATYYCQQYSGYPLTFGAGTKLELKRASAESKYGPPCPPCPFWVLVVVGGVLACYSLLVTVAFIIFWVRSKRSRLLHSDYMNMTPRRLGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - protein, 460 aa, Artificial Sequence
  Exemplary amino acid sequence of CAR PMC882 comprising the TNF alpha scFv, a CD28 hinge, a CD28 transmembrane domain, a CD28 co-stimulatory domain, and a CD3 zeta signalling domain (without leader sequence)
EVQLLESGGGLVQPGGSLRLSCAASGFTFSSYAMSWVRQAPGKGLEWVSSISSTGASTTYADSVKGRFTISRDNSKNTLYLQMNSLRAEDTAVYYCAKGGAAFDYWGQGTLVTVSSGGGGSGGGGSGGGGSDIQMTQSPSSLSASVGDRVTITCRASQSISSYLNWYQQKPGKAPKLLIYSASYLQSGVPSRFSGSGSGTDFTLTISSLQPEDFATYYCQQANNAPTTFGQGTKVEIKASAIEVMYPPPYLDNEKSNGTIIHVKGKHLCPSPLFPGPSKPFWVLVVVGGVLACYSLLVTVAFIIFWVRSKRSRLLHSDYMNMTPRRLGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - protein, 117 aa, Artificial Sequence
  Anti-TNFa scFv clone P3H2 VH
AEVQLLESGGGLVQPGGSLRLSCAASGFTFSSYAMSWVRQAPGKGLEWVSSISSTGASTTYADSVKGRFTISRDNSKNTLYLQMNSLRAEDTAVYYCAKGGAAFDYWGQGTLVTVSS

SEQ ID NO: 163 - protein, 112 aa, Artificial Sequence
  Anti-TNFa scFv clone P3H2 VL
TDIQMTQSPSSLSASVGDRVTITCRASQSISSYLNWYQQKPGKAPKLLIYSASYLQSGVPSRFSGSGSGTDFTLTISSLQPEDFATYYCQQANNAPTTFGQGTKVEIKRAAA

SEQ ID NO: 164 - protein, 78 aa, Artificial Sequence
  Exemplary amino acid sequence of a CD8a hinge
IEVMYPPPYLDNEKSNGTIIHVKGKHLCPSPLFPGPSKPIEVMYPPPYLDNEKSNGTIIHVKGKHLCPSPLFPGPSKP

SEQ ID NO: 165 - protein, 438 aa, Artificial Sequence
  Exemplary amino acid sequence of a CAR comprising IFN-gamma scFv TII 2G1G7, a IgG4 hinge region, a CD28 transmembrane domain, a CD28 co-stimulatory domain, and a CD3 zeta signalling domain (without leader sequence)
QVKLEQSGPGLVAPSQSLSITCTVSGFSLTSYGVHWVRQSPGKGLEWLGVIWAGGSTNYNSALMSRLSISKDNSKSQVFLKMNSLQTDDTAMYYCARRDGNRYYTMDYWGQGASVTVSSGGGGSGGGGSGGGGSENVLTQSPAIMSASPGEKVTMTCRASSSVSSSYLHWYQQKSGGSPKLWIYRTSNLASGVPARFSGSGSGTSYSLTISSVEAEDAATYYCQQYSGYPLTFGAGTKLELKRASAESKYGPPCPPCPFWVLVVVGGVLACYSLLVTVAFIIFWVRSKRSRLLHSDYMNMTPRRLGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 166 - protein, 244 aa, Artificial Sequence
  Exemplary amino acid sequence of a TNF-alpha scFv clone P3H2 - v2
AEVQLLESGGGLVQPGGSLRLSCAASGFTFSSYAMSWVRQAPGKGLEWVSSISSTGASTTYADSVKGRFTISRDNSKNTLYLQMNSLRAEDTAVYYCAKGGAAFDYWGQGTLVTVSSGGGGSGGGGSGGGGSTDIQMTQSPSSLSASVGDRVTITCRASQSISSYLNWYQQKPGKAPKLLIYSASYLQSGVPSRFSGSGSGTDFTLTISSLQPEDFATYYCQQANNAPTTFGQGTKVEIKRAAA